(19) United States
(12) Patent Application Publication
(10) Pub. No.: US 2004/0235798 A1
(43) Pub. Date: Nov. 25, 2004

(54) Compositions and methods for inhibiting prenyltransferases

(76) Inventors: Krishna K Murthi, Cambridge, MA (US); Arthur Kluge, Lincoln, MA (US)

Correspondence Address: ROPES & GRAY LLP, ONE INTERNATIONAL PLACE, BOSTON, MA, 02110-2624, US

(21) Appl. No.: 10/482,830

(22) PCT Filed: Jul. 2, 2002
(86) PCT No.: PCT/US02/20848

Related U.S. Application Data
(60) Provisional application No. 60/303,245, filed on Jul. 5, 2001.

(51) Int. Cl. A61K 31/66

(52) U.S. Cl. 514/114

(57) ABSTRACT

The present invention relates in part to compositions and methods for inhibiting prenyltransferases.

### BACKGROUND OF THE INVENTION

Fungal infections of humans range from superficial conditions that affect the skin, such as those caused by dermatophytes or Candida species, to deeply invasive and often lethal infections (such as candidiasis and cryptococcosis). Pathogenic fungi occur worldwide, although particular species may predominate in certain geographic areas.

In the past 20 years, the incidence of fungal infections has increased dramatically, as have the numbers of potentially invasive species. Indeed, fungal infections, once dismissed as a nuisance, have begun to spread so widely that they are becoming a major concern in hospitals and health departments. Fungal infections occur more frequently in people whose immune system is compromised or suppressed (e.g., because of organ transplantation, cancer chemotherapy, or the human immunodeficiency virus), who have been treated with broad-spectrum antibacterial agents, or who have been subject to invasive procedures (catheters and prosthetic devices, for example). Fungal infections are now important causes of morbidity and mortality of hospitalized patients: the frequency of invasive candidiasis has increased tenfold to become the fourth most common blood culture isolate (Pannuti et al (1992) Cancer 69:2653). Invasive pulmonary aspergillosis is a leading cause of mortality in bone-marrow transplant recipients (Pannuti et al., supra), while Pneumocystis carinii pneumonia is the cause of death in many patients with acquired immunodeficiency syndrome (AIDS) in North America and Europe (Hughes (1991) Pediatr Infect. Dis J. 10:391). Many opportunistic fungal infections cannot be diagnosed by usual blood culture and must be treated empirically in severely immuno-compromised patients (Walsh et al. (1991) Rev. Infect Dis. 13:496).

The fungi responsible for life-threatening infections include Candida species (mainly Candida albicans, followed by Candida tropicalis), Aspergillus species, Cryptococcus neoformans, Histoplasma capsulatum, Coccidioides immitis, Pneumocystis carinji and some zygomycetes. Treatment of deeply invasive fingal infections has lagged behind bacterial chemotherapy.

There are numerous commentators who have speculated on this apparent neglect. See, for example, Georgopapadakou et al. (1994) Science 264:371. First, like mammalian cells, fungi are eukaryotes, and thus agents that inhibit fungal protein, RNA, or DNA biosynthesis may have the same activity in the patient's own cells, producing toxic side effects. Second, life-threatening fingal infections were thought, until recently, to be too infrequent to warrant aggressive research by the pharmaceutical industry. Other factors have included:

(i) Lack of drugs. A drug known as Amphotericin B has become the mainstay of therapy for fungal infection despite side effects so severe that the drug is known as “amphoterrible” by patients. Only a few second-tier drugs exist.

(ii) Increasing resistance. Long-term treatment of oral candidiasis in AIDS patients has begun to breed species resistant to older antifuigal drugs. Several other species of fingi have also begun to exhibit resistance.

(iii) A growing list of pathogens. Species of fingi that once posed no threat to humans are now being detected as a cause of disease in immune-deficient people. Even low-virulence baker's yeast, found in the human mouth, has been found to cause infection in susceptible burn patients.

(iv) Lagging research. Because pathogenic fungi are difficult to culture, and because many of them do not reproduce sexually, microbiological and genetic research into the disease-causing organisms has lagged far behind research into other organisms.

In the past decade, however, more antifungal drugs have become available.

Nevertheless, there are still major weaknesses in their spectra, potency, safety, and pharmacokinetic properties, and accordingly it is desirable to improve the panel of antifungal agents available to the practitioner.

Many potential anti-fungal compounds that show activity against fungal enzymes in cell-free or high-throughput assays are ineffective in whole cell or in vivo assays. Although the reason for this disparity has not been conclusively demonstrated, one possible cause may be the inability of certain compounds to cross the cell wall and cell membrane and enter the cytoplasm of the fungal cell. Accordingly, difficulty in reliably translating high-throughput assay results into therapeutic efficacy remains a significant barrier in the development of alternative anti-fungal drugs.

### SUMMARY OF THE INVENTION

One aspect of the present invention relates to methods for treating or preventing fungal infections and infections involving other eukaryotic parasites of plants or animals, using phenethylazaryl-bearing compounds that are cytotoxic to fungi or other eukaryotic parasites, e.g., by inhibiting the biological activity of a prenyltransferase, CAK1 enzyme, or N-myristoyl transferase. In certain embodiments, the methods inhibit a GGPTase. The present invention also relates to the novel compositions of matter used in such methods. In certain embodiments, the subject inhibitors can be used for the treatment of mycotic infections in animals; as additives in feed for livestock to promote weight gain; as disinfectant formulations; and as in agricultural applications to prevent or treat fungal infection of plants. In preferred embodiments, the practice of the subject method utilizes inhibitors that are selective inhibitors of the fungal or parasitic prenyltransferase relative to any human prenyltransferases. In certain preferred embodiments, the method can be used for treating a nosocomial fungal and skin/wound infection involving fungal organisms, including, among others, the species Aspergillus, Blastomyces, Candida, Coccidioides, Cryptococcus, Epidermophyton, Hendersonula, Histoplasma, Microsporum, Paecilomyces, Paracoccidioides, Pneumocystis, Trichophyton, and Trichosporium. In other preferred embodiments, the method can be used for treating an animal or plant parasites, such as infections involving liver flukes, nematodes or the like.

According to the present invention, treatment using the inhibitors of the present invention comprises the administration of a pharmaceutical composition of the invention in a therapeutically effective amount to an individual in need of such treatment. The compositions may be administered parenierally by intramuscular, intravenous, intraocular, intraperitoneal, or subcutaneous routes; inhalation; orally, topically and intranasally.

### BRIEF DESCRIPTION OF THE DRAWINGS

FIGS. 1-31 present various illustrative reaction schemes for preparing prenyltransferase inhibitors useful in the methods and compositions of the present invention.

### DETAILED DESCRIPTION OF THE INVENTION

In one aspect, the present invention relates to methods for treating and/or preventing fungal infections using compounds that specifically inhibit the biological activity of fungal enzymes involved in cell wall integrity, hyphael formation, and other cellular functions critical to pathogenesis. In particular, it has been observed by us that prenylation of Rho1-like phosphatases by a geranylgeranylproteintransferase (GGPTase) activity can be critical to maintenance of cell wall integrity in yeast. As described in WO 97/38293, prenylation of, inter alia, Rho1-like GTPase(s) is required for sufficient glucan synthase activity. It was demonstrated that the prenylation of Rho1 by GGPTase I is not only critical to cell growth, but inhibition of the prenylation reaction is a potential target for developing a cytotoxic agent for killing various fungi. Moreover, the relatively high divergence between fungal and human GGPTase sequences suggests that selectivity for the fungal GGPTase activity can be obtained to provide antifungal agents having desirable therapeutic indices.

Different substrate specificity among prenyltransferases allow for preparation of inhibitors of the present invention having improved therapeutic indexes. That is, certain inhibitors may inhibit some prenyltransferases and not others. As a result, inhibitors only for prenyltransferases encoded by oncogenes, and not wildtype enzymes, may be employed. Some of the reasons why different prenyltransferases may exhibit different specificity include the following. The β subunits for FTPase and GGPTase I are distinct, and such subunits contribute significantly to the activity of the enzyme. Also, there may be differences in effect for inhibition of GGPTase and FTPase, because geranylgeranyl protein in mammalian cells exceeds that of farnesylated proteins by a factor of about five. Numerous reports, which are detailed below, report that inhibitors of FPTase may not inhibit GGPTase and vice versa. Despite such differences, other reports indicate that FTPase and GGTPase may, in certain circumstances, prenylate the same substrate (Caldwell et al. (1994) Proc. Natl. Acad. Sci. USA 91:1275-1279). It may be possible to inhibit one prenyltransferase selectively, which should allow for improved therapeutic indexes for any inhibitor when administered specifically for any cancer, neoplasm, or aberrant hyperproliferative disorder resulting from mutation of a particular gene encoding a prenyltransferase.

CAK1 is an essential gene in fungus, the inhibition of which has cytotoxic effects (see U.S. application Ser. No. 09/305,929, incorporated herein by reference). In S. cerevisiae, conditional CAK1 mutants arrest with multiple elongated buds, after shifting 37°, and, after 8 hours at the restrictive temperature, Cak1 mutants begin to lyse in a dramatic fashion. This phenotype is terminal after one to two days at the restrictive temperature and cells fail to recover when shifted back to permissive temperatures.

N-Myristoyltransferase inhibitors have recently been shown to demonstrate cytotoxic activity in fungal cells as well (Lodge, J. et al., J Biol Chem. 1998 May 15;273(20): 12482-91).

Many potential anti-fungal agents which are potent in vitro in cell-free assays are ultimately inactive in cell-based assays or when administered to an animal. Presumably, this effect arises because potent test compounds fail to enter the fungal cell. Compounds of one class of anti-fungal agents, azole anti-fungal agents, more reliably show correlation between cell-free and in vivo activities. A number of these compounds are depicted in the scheme below. However, most of these fungal agents are cytostatic, rather than cytotoxic, and thus are generally less effective than cytotoxic agents.

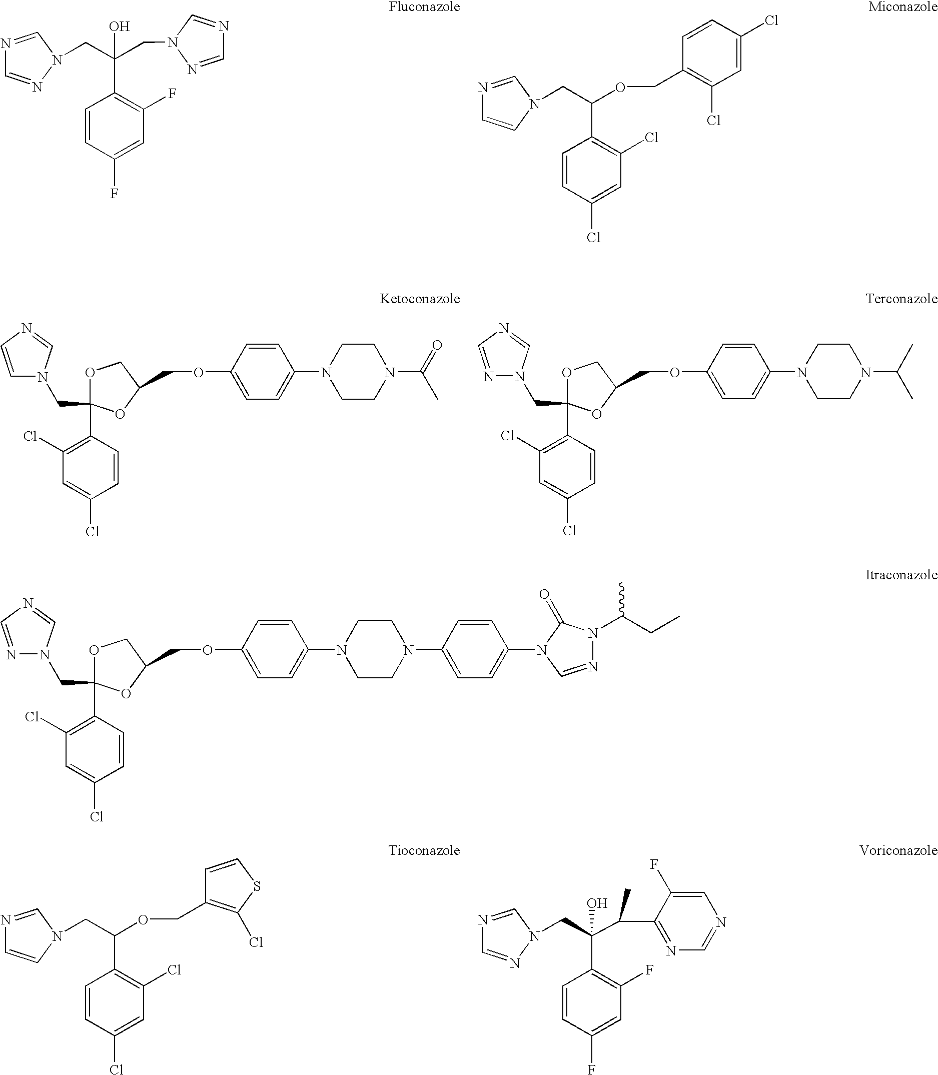

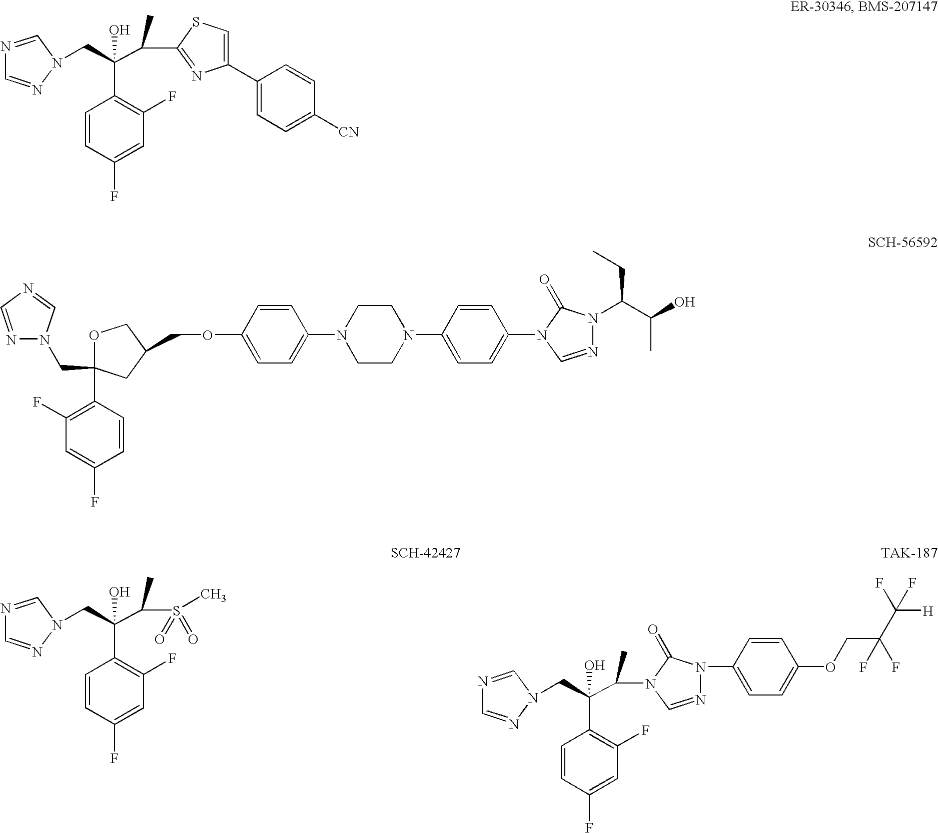

The structural motifs shared by these compounds, referred to herein collectively as a phenethylazaryl portion, are likely to be responsible for the desirable pharmacological characteristics of this class of compounds. Accordingly, covalently linking a phenethylazaryl portion with a cytotoxic antifungal portion should result in a compound that enters fungal cells and is cytotoxic to fungal cells.

Thus, as described in greater detail below, the present invention provides methods and compositions for inhibiting prenyltransferases using small molecule (e.g., less than about 1000 amu) inhibitors that include a phenethylazaryl portion. In the practice of the instant method, the preferred inhibitors inhibit a targeted prenyltransferase with a Kᵢ of 10 μM or less, more preferably 1 μM or less, and even more preferably with a Kᵢ less than 100 nM, 10 nM, or even 1 nM.

In one embodiment, for the treatment of humans or other animals, the subject method preferably employs prenyltransferase inhibitors, such as inhibitors of FPTase, GGPTase I, or GGPTase II to treat cancer, neoplasms and other aberrant hyperproliferative disorders. The chemotherapeutic properties of the compounds of the present invention may be determined from cell-based assays, as well as by other methods, including, inter alia, growth inhibition assays, flow cytometry analyses, and other standard assays known to those skilled in the art. Preferred anticancer agent pharmaceutical preparation, whether for topical, injection or oral delivery (or other route of administration), would provide a dose less than the ED₅₀ for modulation of prenyltansferase activity of nonmutated genes as compared to oncogenic ones, more preferably at least 1 order of magnitude less, more preferably at least 2, 3 or 4 orders of magnitude less.

Another parameter useful in identifying and measuring the effectiveness of the prenyltransferase inhibitor compounds of the invention as anticancer agents is the determination of the kinetics of the activity of such compounds. Such a determination can be made by determining the effect of an inhibitor, e.g., anticancer or antifungal activity, as a function of time. For treatment of fungal infections, in a preferred embodiment, the compounds display kinetics which result in efficient lysis of a fungal cell. In a preferred embodiment, the compounds are fungicidal. For treatment of cancer and other aberrant hyperproliferative disorders, the compounds display kinetics which result in at least slowing of cell proliferation, or more preferably, cell death for any oncogenic cell.

In another embodiment, for the treatment of humans or other animals, the subject method preferably employs prenyltransferase inhibitors which are selective for a fungal enzyme relative to the host animals' prenyltransferase, e.g., the Kᵢ for inhibition of the fungal enzyme is at least one order of magnitude less than the Kᵢ for inhibition any prenyltransferase from human (or other animal), and even more preferably at least two, three, or even four orders of magnitude less. That is, in preferred embodiments, the practice of the subject method in vivo in animals utilizes inhibitors with therapeutic indexes of at least 10, and more preferably at least 100 or 1000. Preferably, inhibitors for use as antifungal agents inhibit fungal GGPTase.

The antifungal properties of the compounds of the present invention may be determined from a fungal lysis assay, as well as by other methods, including, inter alia, growth inhibition assays, fluorescence-based fungal viability assays, flow cytometry analyses, and other standard assays known to those skilled in the art. The assays for growth inhibition of a microbial target can be used to derive an ED₅₀ value for the compound, that is, the concentration of compound required to kill 50% of the fungal sample being tested. Preferred antifungal agent pharmaceutical preparation, whether for topical, injection or oral delivery (or other route of administration), would provide a dose less than the ED₅₀ for modulation of FPTase and/or GGPTase activity in the host (mammal), more preferably at least 1 order of magnitude less, more preferably at least 2, 3 or 4 orders of magnitude less.

Alternatively, growth inhibition by an antifungal compound of the invention may also be characterized in terms of the minimum inhibitory concentration (MIC), which is the concentration of compound required to achieve inhibition of fungal cell growth. Such values are well known to those in the art as representative of the effectiveness of a particular antifungal agent against a particular organism or group of organisms. For instance, cytolysis of a fungal population by an antifungal compound can also be characterized, as described above by the minimum inhibitory concentration, which is the concentration required to reduce the viable fungal population by 99.9%. The value of MIC₅₀, defined as the concentration of a compound required to reduce the viable fungal population by 50%, can also be used. In preferred embodiments, the compounds of the present invention are selected for use based, inter alia, on having MIC values of less than 25 μg/mL, more preferably less than 7 μg/mL, and even more preferably less than 1 μg/mL against a desired fungal target, e.g., Candida albicans.

Furthermore, the preferred antifungal compounds of the invention display selective toxicity to target microorganisms and minimal toxicity to mammalian cells. Determination of the toxic dose (or “LD₅₀”) can be carried out using protocols well known in the field of pharmacology. Ascertaining the effect of a compound of the invention on mammalian cells is preferably performed using tissue culture assays, e.g., the present compounds can be evaluated according to standard methods known to those skilled in that art (see for example Gootz, T. D. (1990) Clin. Microbiol. Rev. 3:13-31). For mammalian cells, such assay methods include, inter alia, trypan blue exclusion and MTT assays (Moore et al. (1994) Compound Research 7:265-269). Where a specific cell type may release a specific metabolite upon changes in membrane permeability, that specific metabolite may be assayed, e.g., the release of hemoglobin upon the lysis of red blood cells (Srinivas et al. (1992) J. Biol. Chem. 267:7121-7127). The compounds of the invention are preferably tested against primary cells, e.g., using human skin fibroblasts (HSF) or fetal equine kidney (FEK) cell cultures, or other primary cell cultures routinely used by those skilled in the art. Permanent cell lines may also be used, e.g., Jurkat cells. In preferred embodiments, the subject compounds are selected for use in animals, or animal cell/tissue culture based at least in part on having LD₅₀'s at least one order of magnitude greater than the MIC or ED₅₀ as the case may be, and even more preferably at least two, three, and even four orders of magnitude greater. That is, in preferred embodiments where the subject compounds are to be administered to an animal, a suitable therapeutic index is preferably greater than 10, and more preferably greater than 100, 1000 or even 10,000.

The invention is also directed to methods for treating a microbial infection in a host using the compositions of the invention. The compounds provided in the subject methods exhibit broad antifungal activity against various fungi and can be used as agents for treatment and prophylaxis of fungal infectious diseases. For instance, the subject method can be used to treat or prevent nosocomial fungal and skin/wound infection involving fungal organisms, including, among others, the species Aspergillus, Blastomyces, Candida, Coccidioides, Cryptococcus, Epidermophyton, Hendersonula, Histoplasma, Microsporum, Paecilomyces, Paracoccidioides, Pneumocystis, Trichophyton, and Trichosporium.

According to the present invention, treatment of such fungal infections comprises the administration of a pharmaceutical composition of the invention in a therapeutically effective amount to an individual in need of such treatment. The compositions may be administered parenterally by intramuscular, intravenous, intraocular, intraperitoneal, or subcutaneous routes; by inhalation; orally, topically or intranasally.

The subject inhibitors of the present invention, and their method of use, may also be used to inhibit neoplastic growth or proliferative disorders in tissue culture. In addition, the subject inhibitors, and corresponding antifungal methods are also particularly useful in inhibiting unwanted fungal growth in tissue culture, especially those used for production of recombinant proteins or vectors for use in gene therapy.

The invention is also directed to pharmaceutical compositions containing one or more of the inhibitory compounds of the invention as the active ingredient which may be administered to a patient. In addition, the invention is also directed to pharmaceutical compositions containing one or more of the antimicrobial compounds of the invention as the active ingredient which may be administered to a host animal.

There have been a number of reports on methods for detecting inhibitors of prenyltransferases and uses of such inhibitors. For example, inhibition of farnesyl-protein transferase has been shown to block the growth of Ras-transformed cells in soft agar and to modify other aspects of their transformed phenotype. It has also been demonstrated that certain inhibitors of farnesyl-protein transferase selectively block the processing of the Ras oncoprotein intracellularly (N. E. Kohl et al. (1993) Science 260:1934-1937; James et al. (1993) Science 260:1937-1942). Recently, it has been shown that an inhibitor of farnesyl-protein transferase blocks the growth of Ras-dependent tumors in nude mice (N. E. Kohl et al. (1994) Proc. Natl. Acad. Sci U.S.A. 91:9141-45) and induces regression of mammary and salivary carcinomas in Ras transgenic mice (N. E. Kohl et al. (1995) Nature Medicine 1:792-797). Inhibition of GGPTase has been shown to lead to G₀/G₁ arrest in fibroblasts (Vogt et al. (1997) J. Biol. Chem. 272:2722-27229). Several antibiotics (UCF1-A through UCF1-C) structurally related to manumycin inhibited growth of Ki-Ras-transformed fibrosarcoma (Hara et al. (1993) Proc. Natl. Acad. Sci. USA 90:2281-2285 ). Burk et al. WO 92/20336 describes nonpeptidyl FPTase inhibitors prepared by modification of natural products.

Various prenyltransferase inhibitors exhibit varying degrees of inhibition of different prenyltransferases (Lerner et al. (1997) Oncogene 15:1283-1288). Some reports describe specific inhibitors of FPTase that do not inhibit GGPTase (Garcia et al. (1993) J. Biol. Chem. 268:18415-18418; Ratemi et al. (1996) J. Org. Chem. 61:6296-6301). Conversely, inhibitors of GGPTase and not FPTase have also been reported (Macchia et al. (1996) J. Med. Chem. 39:1352-1356; Lerner et al. (1995) J. Biol. Chem. 270:26770-26773).

For additional reports on methods for detecting inhibitors of prenyltransferases and uses thereof, see European Patent Applications 0-537 008 A1, 0 621 342 A1, 0 618 221 A2, 0 537 008 B1; Canadian Patent Application 2,143,588; PCT Publications WO 93/24643, WO 95/25086; WO 95/11917, WO 95/20396, WO 95/13059, WO 96/21456, WO/97/30992, WO 97/38664, WO 97/19091, WO 97/17070, WO 97/18813; U.S. Pat. Nos. 5,602,098, 5,470,832, 5,705,686, 5,721,236, 5,532,359, 5,574,025, 5,624,936, 5,510,510; 5,854,264; Bukhtiyarov et al (1995) J. Biol. Chem. 270:19035-19040; Graham et al. (1994) J. Med. Chem. 37:725-732; Hunt et al. (1996) J. Med. Chem. 39:353-358; Leffieris et al. (1994) Bioorganic & Med. Chem. Letts. 4:887-8892.

Other uses of prenyltransferase inhibitors have been reported. For example, it has recently been reported that farnesyl-protein transferase inhibitors are inhibitors of proliferation of vascular smooth muscle cells and are therefore useful in the prevention and therapy of arteriosclerosis and diabetic disturbance of blood vessels (JP H7-112930). In addition, inhibition of protein geranylgeranylation causes a superinduction of nitric-oxide synthase-2 by interleukin-1-beta in vacular smooth muscle cells (Finder et al. (1997) J. Biol. Chem. 272:13484-13488).

I. Definitions

Before further description of the preferred embodiments of the subject invention, certain terms employed in the specification, examples, and appended claims are collected here for convenience.

The terms “aberrant proliferation” and “unwanted proliferation” are interchangeable and refer to proliferation of cells that is undesired, e.g., such as may arise it due to transformation and/or immortalization of the cells, e.g., neoplastic or hyperplastic.

The term “patient” refers to an animal, preferably a mammal, including humans as well as livestock and other veterinary subjects.

The terms “fungi” and “yeast” are used interchangeably herein and refer to the art recognized group of eukaryotic protists known as fungi. That is, unless clear from the context, “yeast” as used herein can encompass the two basic morphologic forms of yeast and mold and dimorphisms thereof.

As used herein, the term “antimicrobial” refers to the ability of the inhibitors of the invention to prevent, inhibit or destroy the growth of microbes such as bacteria, fungi, protozoa and viruses.

The term “prodrug” is intended to encompass compounds which, under physiological conditions, are converted into the inhibitor agents of the present invention. A common method for making a prodrug is to select moieties which are hydrolyzed under physiological conditions to provide the desired biologically active drug. In other embodiments, the prodrug is converted by an enzymatic activity of the patient or alternatively of a target fungi.

The term “ED₅₀” means the dose of a drug which produces 50% of its maximum response or effect. Alternatively, it may refer to the dose which produces a pre-determined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀. The term “structure-activity relationship” or “SAR” refers to the way in which altering the molecular structure of drugs alters their interaction with a receptor, enzyme, etc.

The term “acylamino” is art-recognized and refers to a moiety that can be represented by the general formula:

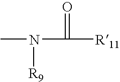

wherein R₉ is as defined above, and R′₁₁ represents a hydrogen, an alkyl, an alkenyl or —(CH₂)ₘ—R₈, where m and R₈ are as defined above.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

The terms “alkenyl” and “alkynyl” refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethylthio, and the like.

The terms “amine” and “amino” are art-recognized and refer to both unsubstituted and substituted amines, e.g., a moiety that can be represented by the general formula:

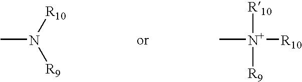

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R10 can be a carbonyl, e.g., R₉, R₁₀ and the nitrogen together do not form an imide. In certain such embodiments, neither R₉ and R₁₀ is attached to N by a carbonyl, e.g., the amine is not an amide or imide, and the amine is preferably basic, e.g., has a pKₐ above 7. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “amido” is art-recognized as an amino-substituted carbonyl and includes a moiety that can be represented by the general formula:

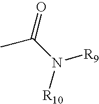

wherein R₉, R₁₀ are as defined above. Preferred embodiments of the amide will not include imides, which may be unstable.

The term “aralkyl”, as used herein, refers to an alkyl group substituted with an aryl group (e.g., an aromatic or heteroaromatic group).

The term “aryl” as used herein includes 5-, 6-, and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphate, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “carbocycle”, as used herein, refers to an aromatic or non-aromatic ring in which each atom of the ring is carbon.

The term “carbonyl” is art-recognized and includes such moieties as can be represented by the general formula:

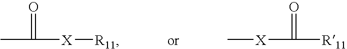

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The term “heteroatom” as used herein means an atom of any element other than carbon or hydrogen. Preferred heteroatoms are boron, nitrogen, oxygen, phosphorus, sulfur and selenium.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

A “phosphonamidite” can be represented in the general formula:

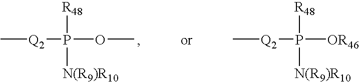

wherein R₉ and R₁₀ are as defined above, Q₂ represents O, S or N, and R₄₈ represents a lower alkyl or an aryl, Q₂ represents O, S or N.

A “phosphoramidite” can be represented in the general formula:

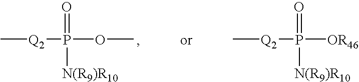

wherein R₉ and R₁₀ are as defined above, and Q₂ represents O, S or N.

A “phosphoryl” can in general be represented by the formula:

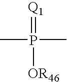

wherein Q₁ represented S or O, and R₄₆ represents hydrogen, a lower alkyl or an aryl. When used to substitute, for example, an alkyl, the phosphoryl group of the phosphorylalkyl can be represented by the general formula:

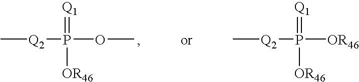

wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. N Protective Groups in Organic Synthesis, 2^{nd} ed.; Wiley: N.Y., 1991).

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₈, m and R₈ being defined above.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

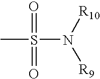

in which R₉ and R₁₀ are as defined above.

The term “sulfate” is art recognized and includes a moiety that can be represented by the general formula:

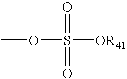

in which R₄₁ is as defined above.

The term “sulfonamido” is art recognized and includes a moiety that can be represented by the general formula:

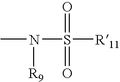

in which R₉ and R′₁₁ are as defined above.

The term “sulfonate” is art-recognized and includes a moiety that can be represented by the general formula:

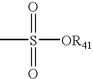

in which R₄₁ is an electron pair, hydrogen, alkyl, cycloalkyl, or aryl.

The terms “sulfoxido” or “sulfinyl”, as used herein, refers to a moiety that can be represented by the general formula:

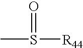

in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, m, n, etc., when it occurs more than once in any structure, is intended to be independent of its definition elsewhere in the same structure.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Certain compounds of the present invention may exist in particular geometric or stereoisomeric forms. The present invention contemplates all such compounds, including cis- and trans-isomers, R— and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., the ability to inhibit hedgehog signaling), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

II. Compounds and Preparations Thereof

The present invention makes available a novel method for designing anti-fungal therapeutics with improved capacity for intracellular localization. According to this method, a molecule having two portions is prepared. One portion is an antifungal subunit, and the other portion is a phenethylazaryl subunit that increases the effectiveness of the compound relative to the antifungal subunit alone, e.g., by increasing cellular uptake of the compound relative to the antifungal subunit alone, and thus increases whole cell activity and activity against a fungal infection in an animal. The antifungal subunit is a structure which, taken alone, exhibits anti-fungal activity (e.g., against a target fungal enzyme in a cell-free assay; for example, has been independently tested for activity) or includes a substructure having a high degree of similarity to a compound which exhibits activity, such as a cytostatic or, preferably a cytotoxic activity, against a target fungal enzyme, such as a prenyltransferase (e.g., a GGTPase or FTPase), in an assay, or an analog of any such structure modified to facilitate attachment of the phenethylazaryl subunit. In preferred embodiments, the antifungal subunit is non-peptidyl, e.g., a small organic molecule. For example, a substructure having a high degree of similarity to an active compound may have a core ring structure (e.g., including one or more covalently linked rings, including fused, bridged, spiro, or separate rings) in common with the active compound, and may have one or more substituents on the core ring structure in corresponding, geminal, or vicinal positions to substituents present on the core ring structure of the active compound. Such substituents are referred to herein as ‘analogous substituents’. An analogous substituent in a corresponding position is bound to the same position on the ring and in the same stereochemical location as a substituent on the active compound. An analogous substitutent in a geminal position is bound to the same position on the ring as a substituent on the active compound, but may be attached in a different stereochemical location. An analogous substituent in a vicinal position is attached to an atom adjacent to the position of the substituent on the active compound, and may have a similar or differing stereochemical disposition. Preferably, an analogous substituent of the substructure has a similar polarity to the substituent of the active compound, e.g., is hydrophobic if the analogous substituent of the active compound is hydrophobic, or hydrophilic if the analogous substituent of the active compound is hydrophilic. A core ring structure of substructure of a compound according to the present invention may have more or fewer substituents than the core ring structure of an active compound.

In certain embodiments, a substructure having a high degree of similarity to an active compound may have a core ring structure which is a nor- or homo-variant of the core ring structure of an active compound. A nor-variant is a core ring structure which includes one or more rings having one fewer atom than the core ring structure of the active compound, e.g., a cyclopentyl in place of a cyclohexyl, or a pyrrolidine in place of a piperidine, etc. A homo-variant is a core ring structure which includes one or more rings having one more atom than the core ring structure of the active compound, e.g., a pyran in place of a furan, or a pyridine in place of a pyrrole. Additionally, the core ring structure of a substructure of a compound according to the present invention may differ by one or more degrees of unsaturation, e.g., may have one or more additional or fewer double bonds in the core ring structure, as compared with the core ring structure of an active compound.

Other strategies and methods for varying the structure of an active compound for use in the anti-fungal portion of a compound as described herein will be readily understood by those of skill in the art, and can be devised, selected, and prepared according to techniques well known in the art of medicinal chemistry.

The phenethylazaryl portion is a subunit having a structure according to the general formula:

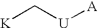

wherein A represents a substituted or unsubstituted aryl or heteroaryl ring;

U represents a carbon or nitrogen atom, preferably an sp³-hybridized carbon atom, to which the linkage is attached; and

K represents a nitrogen-containing heteroaryl ring.

In certain embodiments, A represents a phenyl ring, preferably bearing from 1-3 substituents, even more preferably a disubstituted phenyl ring such as a 2,4-disubstituted phenyl ring. In certain embodiments, A is a phenyl ring substituted with at least one halogen atom. In certain embodiments, the phenyl ring is substituted with a halogen atom at an ortho and a para position.

In certain embodiments, K is a substituted or unsubstituted pyridine, imidazole, pyrrole, or triazole ring, preferably an imidazole or triazole ring. In preferred embodiments, K represents an unsubstituted imidazole or triazole ring linked through a nitrogen atom of the ring.

In certain embodiments, the phenethylazaryl portion has the formula:

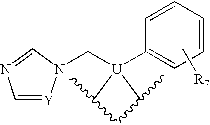

wherein Y represents CH or N;

U represents a nitrogen or carbon atom, such as an sp³-hybridized carbon atom, to which the linkage is attached; and

R₇ represents from 0 to 5 substituents on the ring to which it is attached, preferably from 1 to 3 substitutents, independently selected from fluoro, chloro, bromo, iodo, nitro, and cyano.

In certain embodiments, R₇ includes at least two halogen substituents, e.g., Cl and/or F, preferably located at an ortho and a para position on the phenyl ring. In certain embodiments, R₇ consists of two halogen substituents, e.g., Cl and/or F, located at an ortho and a para position on the phenyl ring.

The anti-fungal portion is linked to the phenethylazaryl portion through a linkage (i.e., the shortest linear route of covalent bonds between the two portions) comprising from 0 to 15 atoms, or from 0 to 10 atoms, or from 0 to 5 atoms. The atoms in the linkage are preferably selected from C, O, S, N, P, and Se, although other divalent, trivalent, or tetravalent atoms, such as B, Si, Se, etc., may optionally be present. In certain embodiments, each position in the linkage is an occurrence of M, as defined below. The linkage may include atoms in a linear chain, a ring, or both. For example, in one embodiment, the linkage includes an N-phenyl-piperazine moiety. The linkage is attached to the phenethylazaryl portion at the carbon atom U, and may be attached to the anti-fungal portion at any position.

A preferred position for attaching the linkage to the anti-fungal portion can be selected by determining a position of the anti-fungal portion which is insensitive to substitution. For example, a series of anti-fungal portions can be prepared as independent molecules (e.g., without attaching a phenethylazaryl portion), wherein a bulky substituent is attached to different positions of a core ring structure, such as an active compound. Anti-fungal activity of the compounds in the series can then be measured, and the decrease in anti-fungal activity will be greatest in compounds where the bulky substituent is attached to a position which is sensitive to substitution, while compounds in which the bulky substituent is attached to a position insensitive to substitution will have activities closest to that of a compound having the analogous structure without the bulky substituent. The difference in sensitivity between different positions may be due to the fact that some positions of an active compound interact more closely with a target protein than others, that attachment of a substitutent to some positions alters the conformation of the core ring structure unfavorably for interaction with the target protein, or any other reason as will be understood to those of skill in the art.

In certain embodiments, U taken together with the linkage to the anti-fungal portion represents one of the following groups:

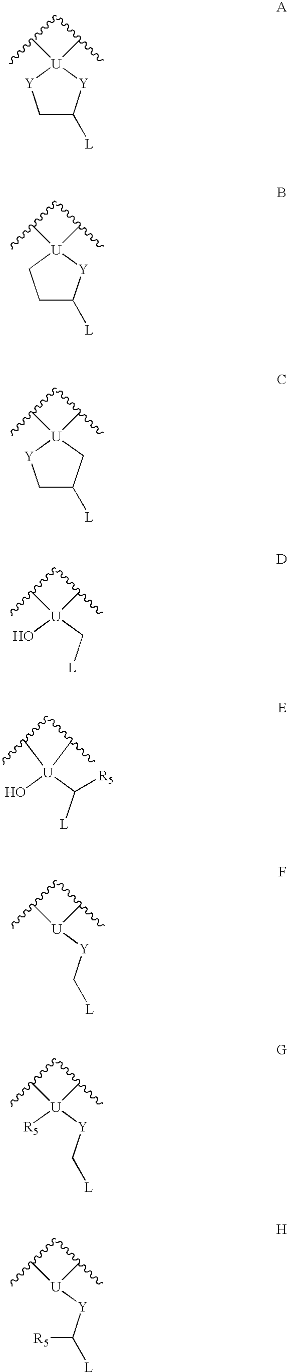

wherein L represents additional atoms of the linkage or a direct bond to the anti-fungal portion;

Y represents, independently for each occurrence, O, S, or Se, preferably O; and

R₅ represents a lower alkyl group, such as methyl or ethyl, preferably methyl.

In certain embodiments, L represents from 0-3 occurrences of M (where M is a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne), such as from 0-2 substituted or unsubstituted methylene groups and, optionally, a heteroatom selected from NR₅, NH, O, S, or Se, attached to the anti-fungal subunit,

wherein R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl.

In certain embodiments, a hydrogen substitutent of one of the groups A-H can be replaced by a methyl, methoxy, or ethyl group, or, if a carbon bearing the hydrogen to be replaced is not bound to an oxygen atom, a hydroxyl group.

In certain embodiments, a subject inhibitor is represented by the general formula I:

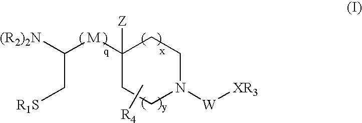

wherein, as valence and stability permit,

W represents —C(═Y)—, —S(O)—, or —S(O)₂—, preferably —C(═Y)—;

X represents O, S, or NR₃, preferably NR₃;

Y is O or S, preferably O;

Z is H or OH;

R₁ represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

R₂, independently for each occurrence, represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

X represents O, S, or NR₃, preferably NR₃;

R₃, independently for each occurrence, represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-anino acid residue), or two R₃ taken together may form a 4- to 8-membered ring, e.g., with N, which ring may include one or more carbonyls and/or heteroatoms;

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR, a subunit selected from —C(═Y)—, —S(O)—, or —S(O)₂—, or two M taken together represent substituted or unsubstituted ethene or ethyne;

q represents an integer from 0 to 3;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain other embodiments, the subject method can be practiced using an inhibitor of a prenyltransferase represented by the general formula II:

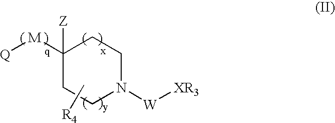

wherein, as valence and stability permit,

Q represents a substituted or unsubstituted heteroaryl moiety containing at least one nitrogen atom in the ring structure, such as a pyridyl or imidazolyl ring;

Ar represents an aryl or heteroaryl ring, e.g., a substituted or unsubstituted phenyl ring;

W represents —C(═Y)—, —S(O)—, or —S(O)₂—, preferably C(═Y)—;

Y is O or S, preferably O;

Z is H or OH;

R₃ represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue);

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain embodiments, Q represents substituted or unsubstituted imidazolyl, oxazolyl, pyrrolyl, pyridyl, or thiazolyl. In embodiments wherein Q represents imidazolyl, Q may be attached to M at nitrogen, or may include an alkyl or aralkyl substituent on nitrogen, e.g., methyl, benzyl, etc. Preferably, Q represents pyridyl, imidazolyl, or N-methylimidazolyl, and may, in certain embodiments wherein Q is imidazolyl, be attached to the subject inhibitor at the 5-position of the imidazole ring. In embodiments wherein Q represents pyridyl, Q may be attached at the meta-position or the para-position, for example.

In certan embodiments, at least one occurrence of R₃ is an aralkyl group, e.g., a substituted or unsubstituted benzyl group. In certain such embodiments wherein two R₃ are present, the other R₃ may represent another aralkyl group or a linkage to a phenethylazaryl subunit. In certain embodiments wherein X is NR₃, both occurrences of R₃ are aralkyl, e.g., substituted or unsubstituted benzyl, groups. In certain embodiments, R₃ may represent a benzyl group substituted with one or more halogens. In certain embodiments wherein X is NR₃, both occurrences of R₃ are identical, e.g., both benzyl, m-chlorobenzyl, etc.

In certain embodiments, R₄ is absent. In certain embodiments, R₄ represents one linkage to a phenethylazaryl subunit or a substituted or unsubstituted alkyl, aryl, heteroaryl, aralkyl, or heteroaralkyl group, e.g., located adjacent to the carbon bound to M.

In certain embodiments, R₁ and R₂ are H for all occurrences.

In certain embodiments, the sum of x and y is one or two. When Z is OH, the occurrence of M bound to the carbon bearing Z preferably represents a substituted or unsubstituted methylene group.

In certain embodiments, the subject method can be practiced using an inhibitor of a prenyltransferase represented by the general formula III:

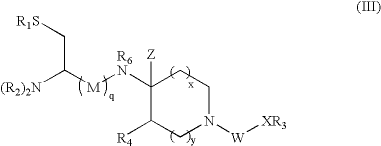

wherein, as valence and stability permit,

X represents O, S, or NR₃, preferably NR₃;

W represents C(═Y)—, —S(O)—, or —S(O)₂—, preferably —(═Y)—;

Y is O or S, preferably O;

Z represents H or OH;

R₁ represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

R₂, independently for each occurrence, represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

R₃, independently for each occurrence, represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue), or two R₃ taken together may form a 4- to 8-membered ring, e.g., with N, which ring may include one or more carbonyls and/or heteroatoms;

R₄ represents a linkage to a phenethylazaryl subunit, H, or substituted or unsubstituted alkyl, aryl, heterocyclyl, aralkyl, heteroaryl, heteroaralkyl, N(₈)₂, OR₈, SR₈, C(═O)R₈, COOR₈, CON(R₈)₂, or an amino acid residue;

R₆ represents a linkage to a phenethylazaryl group, H or substituted or unsubstituted alkyl, alkenyl, alkynyl, cycloalkyl, heterocyclyl, heterocyclylalkyl, aralkyl, aryl, heteroaryl, or heteroaralkyl, preferably H, a linkage to a phenethylazaryl group, or lower alkyl;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3, preferably from 1-2;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain other embodiments, the subject method can be practiced using an inhibitor of a prenyltransferase represented by the general formula IV:

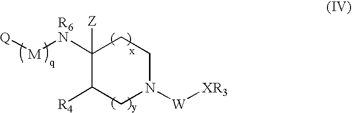

wherein, as valence and stability permit,

Q represents a substituted or unsubstituted heteroaryl moiety containing at least one nitrogen atom in the ring structure, such as a pyridyl or imidazolyl ring;

Ar represents an aryl or heteroaryl ring, e.g., a substituted or unsubstituted phenyl ring;

W represents —C(═Y)—, —S(O)—, or —S(O)₂—, preferably —C(═Y)—;

X represents O, S, or NR₃, preferably NR₃;

Y is O or S, preferably O;

Z represents H or OH;

R₃ represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue);

R₄ represents a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₆ represents a linkage to a phenethylazaryl group, H or substituted or unsubstituted alkyl, alkenyl, alkynyl, cycloalkyl, heterocyclyl, heterocyclylalkyl, aralkyl, aryl, heteroaryl, or heteroaralkyl, preferably H, a linkage to a phenethylazaryl group, or lower alkyl;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3, preferably from 1-2;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain embodiments, Q represents substituted or unsubstituted imidazolyl, oxazolyl, pyrrolyl, pyridyl, or thiazolyl. In embodiments wherein Q represents imidazolyl, Q may be attached to M at nitrogen, or may include an alkyl or aralkyl substituent on nitrogen, e.g., methyl, benzyl, etc. Preferably, Q represents pyridyl, imidazolyl, or N-methylimidazolyl, and may, in certain embodiments, be attached to the subject inhibitor at the 5-position of the imidazole ring. In embodiments wherein Q represents pyridyl, Q may be attached at the meta-position or the para-position, for example.

In certan embodiments, at least one occurrence of R₃ is an aralkyl group, e.g., a substituted or unsubstituted benzyl group. In certain such embodiments wherein two R₃ are present, the other R₃ may represent another aralkyl group or a linkage to a phenethylazaryl subunit. In certain embodiments wherein X is NR₃, both occurrences of R₃ are aralkyl, e.g., substituted or unsubstituted benzyl, groups. In certain embodiments, R₃ may represent a benzyl group substituted with one or more halogens. In certain embodiments wherein X is NR₃, both occurrences of R₃ are identical, e.g., both benzyl, m-chlorobenzyl, etc.

In certain embodiments, R₄ is absent. In certain embodiments, R₄ is a linkage to a phenethylazaryl subunit, or a substituted or unsubstituted alkyl, aryl, heteroaryl, aralkyl, or heteroaralkyl group, e.g., located adjacent to the carbon bound to M.

In certain embodiments, R₁ and R₂ are H for all occurrences.

In certain embodiments, the sum of x and y is one or two. In certain such embodiments, y is at least 1. When Z is OH, the occurrence of M bound to the carbon bearing Z preferably represents a substituted or unsubstituted methylene group.

In certain embodiments, the subject method can be practiced using an inhibitor of a prenyltransferase represented by the general formula V:

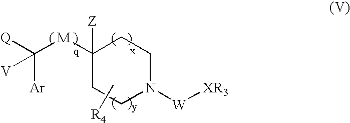

wherein, as valence and stability permit,

Q represents a substituted or unsubstituted heteroaryl moiety containing at least one nitrogen atom in the ring structure, such as a pyridyl or imidazolyl ring;

Ar, independently for each occurrence, represents an aryl or heteroaryl ring, e.g., a substituted or unsubstituted phenyl ring;

V is H or OH;

W represents —C(═Y)—, —S(O)—, or —S(O)₂—, preferably —C(═Y)—;

X represents O, S, or NR₃, preferably NR₃;

Y is O or S, preferably O;

Z represents H or OH;

R₁ represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

R₂, independently for each occurrence, represents H or substituted or unsubstituted alkyl, alkenyl, alkynyl, cycloalkyl, heterocyclyl, heterocyclylalkyl, aralkyl, aryl, heteroaryl, or heteroaralkyl, preferably H or lower alkyl;

R₃, independently for each occurrence, represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue), or two R₃ taken together may form a 4- to 8-membered ring, e.g., with N, which ring may include one or more carbonyls and/or heteroatoms;

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3, preferably from 1-2;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain other embodiments, the subject method can be practiced using an inhibitor of a prenyltransferase represented by the general formula VI:

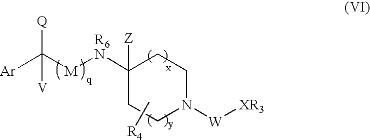

wherein, as valence and stability permit,

Q represents a substituted or unsubstituted heteroaryl moiety containing at least one nitrogen atom in the ring structure, such as a pyridyl or imidazolyl ring;

Ar represents an aryl or heteroaryl ring, e.g., a substituted or unsubstituted phenyl ring;

V is H or OH;

W represents —C(═Y)—, —S(O)—, or —S(O)₂—, preferably —C(═Y)—;

X represents O, S, or NR₃, preferably NR₃;

Y is O or S, preferably O;

Z represents H or OH;

R₃ represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue), or two R₃ taken together may form a 4- to 8-membered ring, e.g., with N, which ring may include one or more carbonyls and/or heteroatoms;

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₆ represents a linkage to a phenethylazaryl group, H or substituted or unsubstituted alkyl, alkenyl, alkynyl, cycloalkyl, heterocyclyl, heterocyclylalkyl, aralkyl, aryl, heteroaryl, or heteroaralkyl, preferably H, a linkage to a phenethylazaryl group, or lower alkyl;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3, preferably from 1-2;

x and y represent, independently, 0, 1, or 2; and

n, individually for each occurrence, represents an integer from 0 to 10, preferably from 0 to 5,

wherein at least one linkage to a phenethylazaryl subunit is present, preferably only one.

In certain embodiments, Q represents substituted or unsubstituted imidazolyl, oxazolyl, pyrrolyl, pyridyl, or thiazolyl. In embodiments wherein Q represents imidazolyl, Q may be attached to the carbon bearing M at nitrogen, or may include an alkyl or aralkyl substituent on nitrogen, e.g., methyl, benzyl, etc. Preferably, Q represents pyridyl, imidazolyl, or N-methylimidazolyl, and may, in certain embodiments, be attached to the subject inhibitor at the 5-position of the imidazole ring. In embodiments wherein Q represents pyridyl, Q may be attached at the meta-position or the para-position, for example.

In certain embodiments, Ar bound to the carbon bearing Q represents a substituted or unsubstituted aryl ring, such as a benzene ring. In certain embodiments, Ar bound to the carbon bearing Q includes at least two aryl rings, e.g., fused (such as naphthyl), linked (such as biphenyl), or tethered (such as a diphenyl ether or diphenyl amine, etc.).

In certan embodiments, at least one occurrence of R₃ is an aralkyl group, e.g., a substituted or unsubstituted benzyl group. In certain such embodiments wherein two R₃ are present, the other R₃ may represent another aralkyl group or a linkage to a phenethylazaryl subunit. In certain embodiments wherein X is NR₃, both occurrences of R₃ are aralkyl, e.g., substituted or unsubstituted benzyl, groups. In certain embodiments, R₃ may represent a benzyl group substituted with one or more halogens. In certain embodiments wherein X is NR₃, both occurrences of R₃ are identical, e.g., both benzyl, m-chlorobenzyl, etc.

In certain embodiments, the sum of x and y is one or two. When Z is OH, the occurrence of M bound to the carbon bearing Z preferably represents a substituted or unsubstituted methylene group. When V is OH, the occurrence of M bound to the carbon bearing V preferably represents a substituted or unsubstituted methylene group.

In certain embodiments, R₁ and R₂ are H for all occurrences.

In certain embodiments, R₄ is absent. In certain embodiments, R represents a linkage to a phenethylazaryl subunit or one substituted or unsubstituted alkyl, aryl, heteroaryl, aralkyl, or heteroaralkyl group, e.g., located adjacent to the carbon bound to M.

In certain embodiments, the subject method can be practiced using an inhibitor of a prenyltransferase represented by the general formula VII:

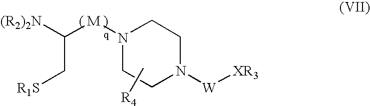

wherein, as valence and stability permit,

W represents —C(═Y)—, —S(O)—, or —S(O)₂—, preferably —C(═Y)—;

Y is O or S, preferably O;

R₁ represents H or substituted or unsubstituted alkyl, alkenyl, alkynyl, cycloalkyl, heterocyclyl, heterocyclylalkyl, aralkyl, aryl, heteroaryl, heteroaralkyl;

R₂, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

X represents O, S, or NR₃, preferably NR₃;

R₃, independently for each occurrence, represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue), or two R₃ taken together may form a 4- to 8-membered ring, e.g., with N, which ring may include one or more carbonyls and/or heteroatoms;

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3; and

n, individually for each occurrence, represents an integer from 0 to 10, preferably from 0 to 5,

wherein at least one linkage to a phenethylazaryl subunit is present, preferably only one.

In certain other embodiments, the subject method can be practiced using an inhibitor of a prenyltransferase represented by the general formula VIII:

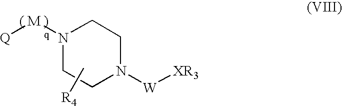

wherein, as valence and stability permit,

Q represents a substituted or unsubstituted heteroaryl moiety containing at least one nitrogen atom in the ring structure, such as a pyridyl or imidazolyl ring;

Ar represents an aryl or heteroaryl ring, e.g., a substituted or unsubstituted phenyl ring;

W represents —C(═Y)—, —S(O)—, or —S(O)₂—, preferably —C(═Y)—;

Y is O or S, preferably O;

X represents O, S, or NR₃, preferably NR₃;

R₃ represents a linkage to a phenethylazaryl subunit, H, substituted or unsubstituted lower alkyl, lower alkenyl, lower alkynyl, cycloalkyl, cycloalkylalkyl, e.g., —(CH₂)ₙcycloalkyl (e.g., substituted or unsubstituted), heterocyclyl, heterocyclylalkyl, e.g., —(CH₂)ₙheterocyclyl (e.g., substituted or unsubstituted), aryl, aralkyl, e.g., —(CH₂)ₙaryl (e.g., substituted or unsubstituted), heteroaryl, heteroaralkyl, e.g., —(CH₂)ₙheteroaryl (e.g., substituted or unsubstituted), or a natural or unnatural amino acid residue (e.g., an alpha-amino acid residue);

R₄ represents, as valency permits, from 0 to 8 substituents on the ring to which it is attached, selected from a linkage to a phenethylazaryl subunit, H, or substituted or unsubstituted alkyl, aryl, heterocyclyl, aralkyl, heteroaryl, heteroaralkyl, N(R₈)₂, OR₈, SR₈, C(═O)R₈, COOR₈, CON(R₈)₂, or an amino acid residue;

R₈, independently for each occurrence, represents H or substituted or unsubstituted alkyl, alkenyl, alkynyl, cycloalkyl, heterocyclyl, heterocyclylalkyl, aralkyl, aryl, heteroaryl, heteroaralkyl;

M represents, independently for each occurrence, a substituted or unsubstituted methylene group, such as —CH₂—, —CHF—, —CHOH—, —CH(Me)—, —C(═O)—, etc., a heteroatom selected from O, S, or NR₈, a subunit selected from —C(═Y)—, —S(O)—, or —S(O)₂—, or two M taken together represent substituted or unsubstituted ethene or ethyne;

q represents an integer from 0 to 3; and

n, individually for each occurrence, represents an integer from 0 to 10, preferably from 0 to 5,

wherein at least one linkage to a phenethylazaryl subunit is present, preferably only one.

In certain embodiments, Q represents substituted or unsubstituted imidazolyl, oxazolyl, pyrrolyl, pyridyl, or thiazolyl. In embodiments wherein Q represents imidazolyl, Q may be attached to M at nitrogen, or may include an alkyl or aralkyl substituent on nitrogen, e.g., methyl, benzyl, etc. Preferably, Q represents pyridyl, imidazolyl, or N-methylimidazolyl, and may, in certain embodiments wherein Q is imidazolyl, be attached to the subject inhibitor at the 5-position of the imidazole ring. In embodiments wherein Q represents pyridyl, Q may be attached at the meta-position or the para-position, for example.

In certan embodiments, at least one occurrence of R₃ is an aralkyl group, e.g., a substituted or unsubstituted benzyl group. In certain such embodiments wherein two occurrences of R₃ are present, the second occurrence of R₃ may represent an aralkyl group or a linkage to a phenethylazaryl subunit. In certain embodiments wherein X represents NR₃, both occurrences of R₃ are aralkyl, e.g., substituted or unsubstituted benzyl, groups. In certain embodiments, R₃ may represent a benzyl group substituted with one or more halogens. In certain embodiments wherein X represents NR₃, both occurrences of R₃ are identical, e.g., both benzyl, m-chlorobenzyl, etc.

In certain embodiments, an occurrence of M directly bound to nitrogen represents —C(═Y)—, CH₂, —S(O)—, or —S(O)₂—, e.g., —C(═O)—, while in other embodiments, the occurrence of M bound to N represents CH₂, or an alkyl-substituted methylene group. In certain embodiments, occurrences of M not bound to nitrogen represent CH₂ or an alkyl-substituted methylene group.

In certain embodiments, R₄ is absent. In certain embodiments, R₄ represents a linkage to a phenethylazaryl subunit, or one substituted or unsubstituted alkyl, aryl, heteroaryl, aralkyl, or heteroaralkyl group, e.g., located adjacent to the nitrogen bound to M. In certain embodiments of Formula VII where R₄ is present at this position, the composition is substantially pure or enriched in the diastereomer wherein the stereocenter where R₄ is attached has the S designation.

In certain embodiments, R₁ and R₂ are H for all occurrences.

In certain embodiments, a subject compound is substantially pure or enriched in one or more diastereomers of the above-described compounds. In certain embodiments of Formula VII, the compound is substantially pure or enriched in a diastereomer wherein the stereocenter where M is attached to the nitrogen- and sulfur-bearing substituent is R.

Exemplary compounds found to inhibit fungal CAK1 enzymes include the following:

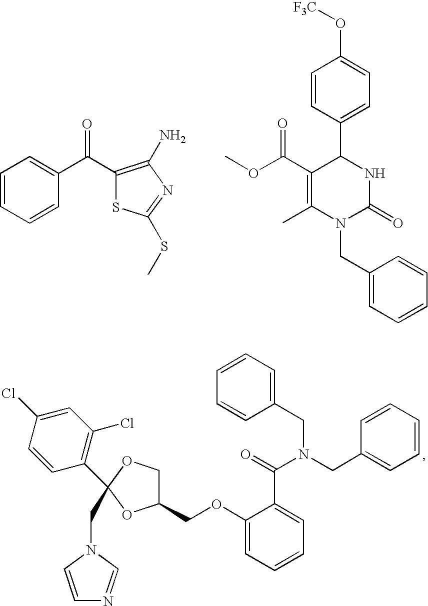

and thus classes of structures which may be useful for inhibiting CAK1 enzymes include analogs and variations of these core structures.

For example, suitable compounds for use in the methods and compositions of the present invention include those having a structure of the formula:

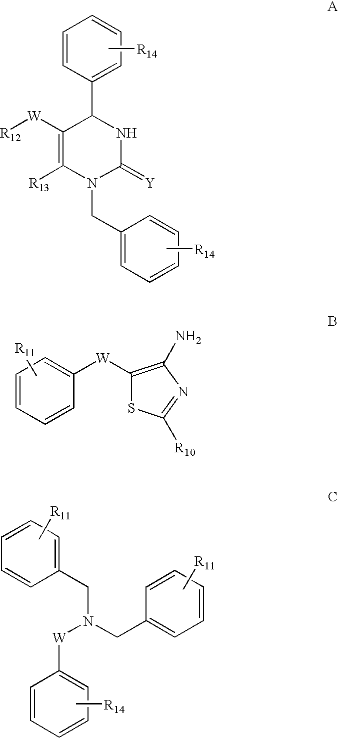

wherein, as valence and stability permit,

W, independently for each occurrence, represents —C(═Y)—, —S(O)—, or —S(O)₂—, preferably —C(═Y)—;

X represents O, S, or NR₃;

Y, independently for each occurrence, is O or S, preferably O;

R₁₀ represents a linkage to a phenethylazaryl group;

R₁₁ represents, independently for each occurrence, from 1-5 substituents on the ring to which it is attached, preferably selected from H, halogen, alkoxy (including haloalkoxy), carboxyl, and a linkage to a phenethylazaryl group;

R₁₂ represents a lower alkyl or a linkage to a phenethylazaryl group;

R₁₃ represents H or lower alkyl; and

R₁₄ represents, independently for each occurrence, from 1-5 substituents on the ring to which it is attached, preferably selected from H, halogen, alkoxy (including haloalkoxy), and carboxyl,

wherein the compound includes at least one phenethylazaryl group, preferably a single one.

In certain embodiments, R₁₁ includes from 1-3 halogen atoms located at ortho and/or para positions on the phenyl ring to which it is attached, e.g., wherein the halogen atoms are the same or are different.

The following compound has been identified as a potent inhibitor of fungal N-myristoyltransferase:

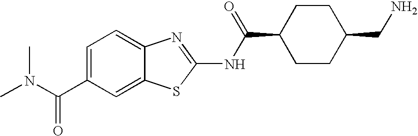

(Abstract # 349, Division of Medicinal Chemistry, American Chemical Society, 221^{st} National Meeting, Apr. 1-5, 2001), and thus structures which may be useful for inhibiting N-myristoylase enzymes include analogs and varations of this structure. Other N-myristoyltransferase inhibitors are disclosed in Devadas, Balekudru, et al. J. Med. Chem. (1995), 38(11), 1837-40; Devadas, Balekudru, et al. Bioorg. Med. Chem. Lett. (1996), 6(16), 1977-1982; Brown, David L. et al. Bioorg. Med. Chem. Lett. (1997), 7(3), 379-382; Lodge, Jennifer K. et al., Microbiology (Reading, U. K.) (1997), 143(2), 357-366; Sikorski, James A. et al., Biopolymers (1997), 43(1), 43-71; Devadas, Balekudru et al., J. Med. Chem. (1997), 40(16), 5 2609-2625; Devadas, Balekudru et al., J. Med. Chem. (1998), 41(6), 996-1000; Lodge, Jennifer K. et al., J. Biol. Chem. (1998), 273(20), 12482-12491; Karki, Rajeshri G. et al. Eur. J. Med. Chem. (2001), 36(2), 147-163; and PCT Application WO 00/37464.

For example, suitable compounds include those having a structure of the formula:

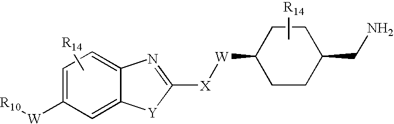

wherein, as valence and stability permit,

W, independently for each occurrence, represents —C(═Y)—, —S(O)—, or —S(O)₂—, preferably —(═Y)—;

X represents O, S, or NR₃;

Y, independently for each occurrence, is O or S, preferably O;

R₁₀ represents a linkage to a phenethylazaryl group; and

R₁₄ represents, independently for each occurrence, from 1-5 substituents on the ring to which it is attached, preferably selected from H, halogen, alkoxy (including haloalkoxy), and carboxyl,

wherein the compound includes at least one phenethylazaryl group, preferably a single one.

Accordingly, the present invention contemplates the preparation and use of compounds such as:

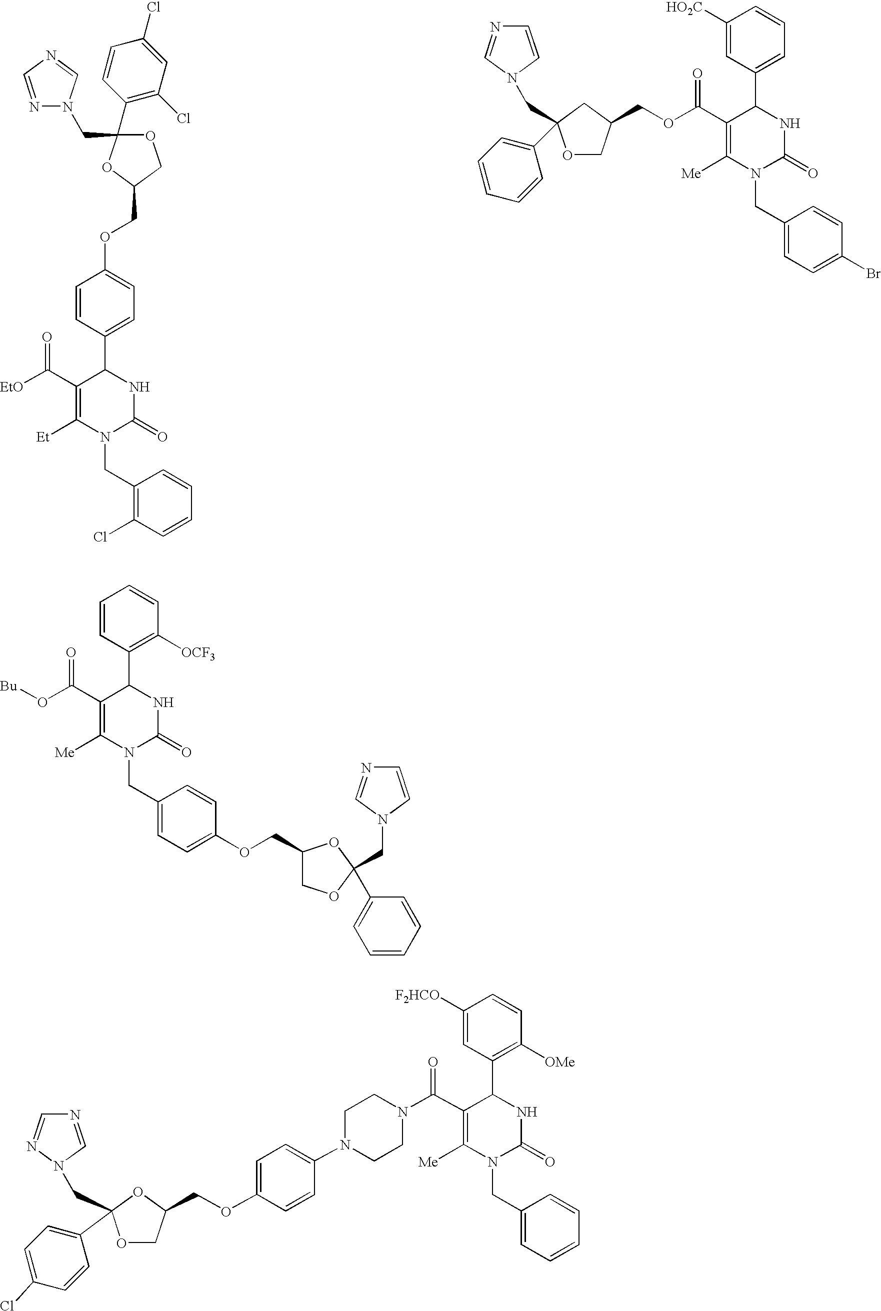

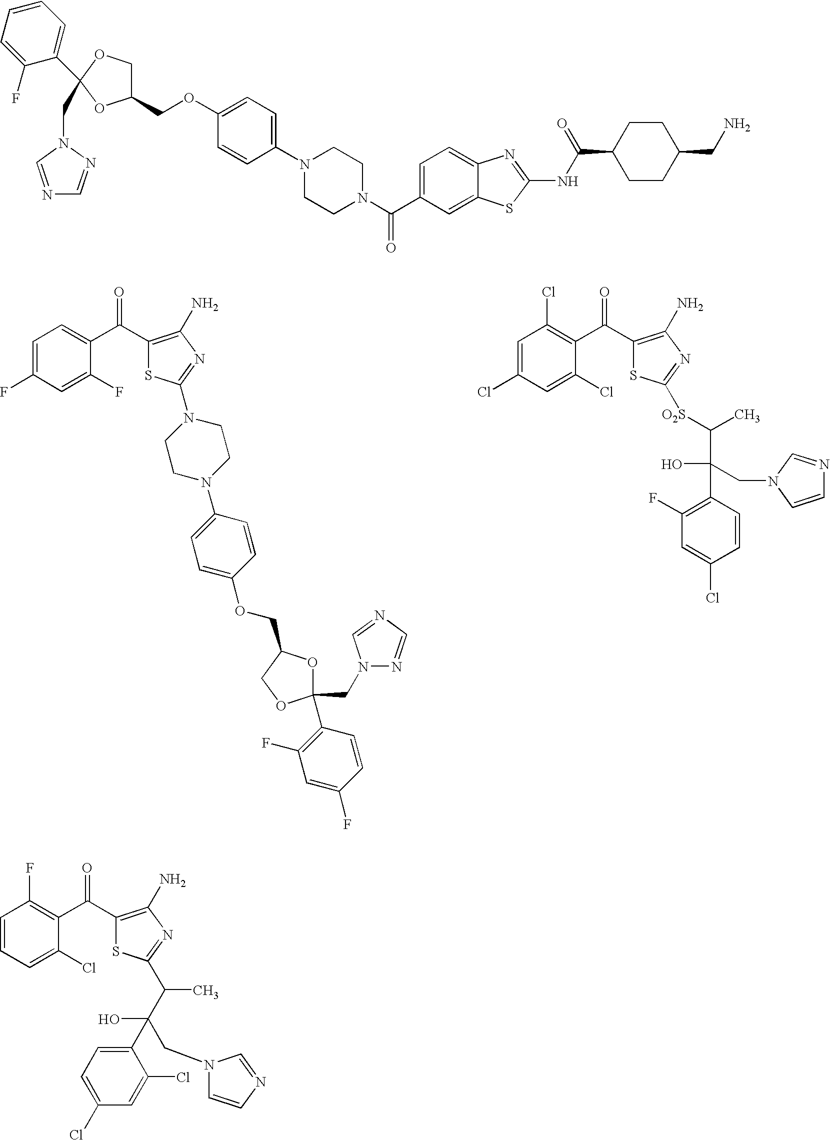

Permease Tags

In certain embodiments, the ability of fungal cells to transport ectopically added compounds, paricularly inhibitors of the present invention, can be enhanced by conjugation of the compound with an amino acid residue or oligopeptide (preferably a dipeptide or tripeptide) which is itself taken up by the a cell in a permease-mediated transport mechanism. Thus, another aspect of the invention features prenyltransferase inhibitors that include a “permease tag”, e.g., which comprises an amino acid residue, dipeptide or tripeptide that facilitates permease-mediated transport of the inhibitor into the fungal pathogen. Such compounds can have desirable pharmacokinetic properties due to, for example, increased bioavailability and/or increased selectivity. With regard to the latter, in preferred embodiments, the permease tag does not increase the cellular uptake of the inhibitor by mammalian cells to any greater degree than it does for cellular uptake by the fungal pathogen, though in the most preferred embodiments, the permease tag increases the uptake by fungal cells to a greater degree than for uptake by mammalian cells.

In other embodiments, the permease tag is removed from the inhibitor as a result of its permease-mediated transport into the fungal pathogen.

In other embodiments, the amino acid or oligopeptide of the permease tag includes a free N-terminal amine, or a group hydrolyzable thereto under the conditions that the pathogen is contacted with the inhibitor.

As demonstrated in the appended examples, in one embodiment the permease tag facilitates permease-mediated transport by an alanine transporter of the fungal pathogen. For example, the inhibitor is derivatized at a free amine with L-alanine, or a dipeptide or tripeptide including L-alanine. In preferred embodiments, the L-alanine moiety is attached to the prenyltransferase inhibitor through an amide linkage through either an amine or carboxyl group of the inhibitor, and provides the complementary functionality in the permease tag. For instance, the L-alanine containing permease tag is provided by derivatization of a free amine on the inhibitor with a carboxyl group on an L-alanine containing oligopeptide, with the oligopeptide providing a free amine (or a group which is hydrolyzable thereto)

Other Candida permeases are known in the art, and appropriate permease tags can be generated for facilitating uptake of the subject inhibitors by other permease-mediated mechanisms. For instance, the permease tag can be selected to increase uptake of the inhibitor by any one of the following Candida permeases:

| Reference | Permease |
|---|---|
| Mukherjee et al. (1998) Yeast | Arginine permease |
| 14:335-45 | |
| Matijekova et al. (1997) | Candida albicans CAN1 gene, encoding a |
| FEBS Lett 408:89-93 | high-affinity permease for arginine, |
| | lysine and histidine |
| Jethwaney et al. (1997) | Proline permease |
| Microbiology 143:397 | |
| Grobler et al. (1995) | mae 1 gene, permease for malate and |
| Yeast 11:1485 | other C4 dicarboxylic acids |
| Gupta et al. (1995) FEMS | purine permease |
| Microbiol Lett 126:93 | |
| Sychrova et al. | lysine-permease |
| (1993) Curr Genet 24:487 | |

Moreover, many more permeases have been identified in S. cervesiae through various genomic projects. Applicants contemplate that the subject permease tags can be selected to increase permease-mediated uptake by a mechanism relying on a Candida homolog of any one of the following S. cerevisae permeases:

| Cerevisae gene | transporter activity |
|---|---|
| AGP1 | asparagine and glutamine permease |
| DIP5 | dicarboxylic amino acid permease |
| MUP1 | high affinity methionine permease |
| TAT2 | high affinity tryptophan transport protein |
| GNP1 | high-affinity glutamine permease |
| ALP1 | high-affinity permease for basic amino acids |
| HIP1 | histidine permease |
| STP4 | involved in pre-tRNA splicing and in uptake of branched- |
| | chain amino acids |
| BAP2 | leucine permease, high-affinity (S1) |
| LYP1 | lysine-specific high-affinity permease |
| ARG11 | member of the mitochondrial carrier family (MCF) |
| PUT4 | proline and gamma-aminobutyrate permease |
| BAP3 | valine transporter |

Pharmaceutical Compositions

In another aspect, the present invention provides pharmaceutically acceptable compositions which comprise a therapeutically effective amount of one or more compounds of the subject invention, such as described above, formulated together with one or more pharmaceutically acceptable carriers (additives) and/or diluents for use in the treatment of fungal infections. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intravectally, for example, as a pessary, cream or foam. In certain embodiments, the pharmaceutical preparations may be non-pyrogenic, i.e., do not elevate the body temperature of a patient.

The phrase “therapeutically effective amount” as used herein means that amount of a compound, material, or composition comprising an inhibitor of the subject invention which is effective for producing some desired therapeutic effect. Such therapeutic effect may result from, for example, inhibition of aberrant hyperproliferation of a cell resulting from transformation of a Ras-related gene, or alternatively, by inhibiting fungal cell wall biosynthesis.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present subject compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of such inhibitors of prenyltransferases. These salts can be prepared in situ during the final isolation and purification of the compounds of the present invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of an inhibitor of prenyltransferases. These salts can likewise be prepared in situ during the final isolation and purification of the compounds of the present invention, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of inhibitor which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association an inhibitor of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. An inhibitor of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulations so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active inhibitor(s) of the present invention, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active inhibitor.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active prenyltransferase inhibitor, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the inhibitor of the present invention in the proper medium. Absorption enhancers can also be used to increase the flux of the drug across the skin. The rate of such flux can be controlled by either providing a rate-controlling membrane or dispersing the compound of the present invention in a polymer matrix or gel.

Ophthalmic formulations, eye ointments, powders, solutions and the like, are also contemplated as being within the scope of this invention.

Pharmaceutical compositions of this invention suitable for parenteral administration comprise one or more inhibitors of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the therapeutic effect of an inhibitor, it is desirable to slow the absorption of the inhibitor from subcutaneous or intramuscular injection. This may be accomplished by the use of a liquid suspension of crystalline or amorphous material having poor water solubility. The rate of absorption of the inhibitor then depends upon its rate of dissolution which, in turn, may depend upon crystal size and crystalline form. Alternatively, delayed absorption of a parenterally administered inhibitor form is accomplished by dissolving or suspending the inhibitor in an oil vehicle.

Injectable depot forms are made by forming microencapsuled matrices of the subject inhibitors in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the prenyltransferase inhibitors useful in the subject method may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response, e.g., antifungal or anticancer activity, for a particular patient, composition, and mode of administration, without being toxic to the patient.

The selected dosage level will depend upon a variety of factors including the activity of the particular prenyltransferase inhibitor employed, or the ester, salt or amide thereof, the route of administration, the time of administration, the rate of excretion of the particular compound being employed, the duration of the treatment, other drugs, compounds and/or materials used in combination with the particular inhibitor employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a potent inhibitor, e.g., having an EC₅₀ in the range of 1 mM to sub-nanomolar, will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated antifungal effects, will range from about 0.0001 to about 1000 mg per kilogram of body weight per day, though preferably 0.5 to 300 mg per kilogram.

If desired, the effective daily dose of the active inhibitor may be administered as two, three, four, five, six or more sub-doses administered separately at appropriate intervals throughout the day, optionally, in unit dosage forms.

In a preferred embodiment, the inhibitor agent is formulated for oral administration, as for example in the form of a solid tablet, pill, capsule, caplet or the like (collectively hereinafter “tablet”) or an aqueous solution or suspension. The inhibitor agent of the present invention may be, for example, an anticancer agent or an antifungal agent. In a preferred embodiment of the tablet form of the inhibitor agent, the tablets are preferably formulated such that the amount of inhibitor agent (or inhibitor agents) provided in 20 tablets, if taken together, would provide a dose of at least the median effective dose (ED₅₀), e.g., the dose at which at least 50% of individuals exhibited a therapeutic affect. For example, for an antifungal agent, the therapeutic effect would be a quantal effect of inhibition of fungal cell growth or protection (e.g., a statistically significant reduction in infection). More preferably, the tablets are formulated such that the total amount of inhibitor agent (or inhibitor agents) provided in 10, 5, 2 or 1 tablets would provide at least an ED₅₀ dose to a patient (human or non-human mammal). In other embodiments, the amount of inhibitor agent (or inhibitor agents) provided in 20, 10, 5 or 2 tablets taken in a 24 hour time period would provide a dosage regimen providing, on average, a mean plasma level of the inhibitor agent(s) of at least the ED₅₀ concentration (the concentration for 50% of maximal effect of, e.g., inhibiting fingal cell growth), though preferably less than 100 times the ED₅₀, and even more preferably less than 10 or 5 times the ED₅₀. In preferred embodiments, a single dose of tablets (1-20 tablets) provides about 0.25 mg to 1250 mg of an inhibitor agent(s).

Likewise, the inhibitor agents can be formulated for parenteral administration, as for example, for subcutaneous, intramuscular or intravenous injection, e.g., the inhibitor agent can be provided in a sterile solution or suspension (collectively hereinafter “injectable solution”). The injectable solution is preferably formulated such that the amount of antifungal agent (or antifungal agents) provided in a 200 cc bolus injection would provide a dose of at least the median effective dose, though preferably less than 100 times the ED₅₀, and even more preferably less than 10 or 5 times the ED₅₀. More preferably, the injectable solution is formulated such that the total amount of antifungal agent (or antifungal agents) provided in 100, 50, 25, 10, 5, 2.5, or 1 cc injections would provide an ED₅₀ dose to a patient, and preferably less than 100 times the ED₅₀, and even more preferably less than 10 or 5 times the ED₅₀. In other embodiments, the amount of inhibitor agent (or inhibitor agents) provided in a total volume of 100 cc, 50, 25, 5 or 2 cc to be injected at least twice in a 24 hour time period would provide a dosage regimen providing, on average, a mean plasma level of the inhibitor agent(s) of at least the ED₅₀ concentration, though preferably less than 100 times the ED₅₀, and even more preferably less than 10 or 5 times the ED₅₀. In preferred embodiments, a single dose injection provides about 0.25 mg to 1250 mg of inhibitor agent.

For continuous intravenous infusion, e.g., drip or push, the inhibitor agent may be provided in a sterile dilute solution or suspension (collectively hereinafter “i.v. injectable solution”). The i.v. injectable solution is preferably formulated such that the amount of inhibitor agent (or inhibitor agents) provided in a 1 L solution would provide a dose, if administered over 15 minutes or less, of at least the median effective dose, though preferably less than 100 times the ED₅₀, and even more preferably less than 10 or 5 times the ED₅₀. More preferably, the i.v. injectable solution is formulated such that the total amount of inhibitor agent (or inhibitor agents) provided in 1 L solution administered over 60, 90, 120 or 240 minutes would provide an ED₅₀ dose to a patient, though preferably less than 100 times the ED₅₀, and even more preferably less than 10 or 5 times the ED₅₀. In preferred embodiments, a single i.v. “bag” provides about 0.25 mg to 5000 mg of inhibitor agent per liter i.v. solution, more preferably 0.25 mg to 2500 mg, and even more preferably 0.25 mg to 1250 mg.

As discussed above, the preferred antifungal agent pharmaceutical preparation, whether for injection or oral delivery (or other route of administration), would provide a dose less than the ED₅₀ for modulation of FPTase, GGPTase, and/or other prenyltransferase activity in the host, more preferably at least 1 order of magnitude less, and more preferably at least 2, 3 or 4 orders magnitude less.

An ED₅₀ dose, for a human, is based on a body weight of from 10 lbs to 250 lbs, though more preferably for an adult in the range of 100 to 250 lbs.

Potential inhibitors may be assessed for ED₅₀ values for any inhibition, incluing for example anticancer or antifungal activity, using any of a number of well known techniques in the art.

III Identifying Candidate Inhibitor Agents

There are a variety of assay formats for testing compounds of the present invention for appropriate inhibition of prenyltransferase activity, CAK1 activity, or N-myristoyltransferase activity. For use as antifungal agents, the inhibitors that may be selected for use in the subject method may be better inhibitors, on the order of magnitudes, of a fungal GGPTase or other prenyltransferase than a mammalian GGPTase or other prenyltransferase, and/or have greater membrane permeance through a fungal cell wall than a mammalian cell membrane.

In general, compositions of matter of the present invention that are candidate inhibitors, for example, of a prenyltransferase, will be screened for activity in appropriate assays. Compounds that display desired characteristics in a given assay may serve as lead compounds for the discovery of more potent inhibitors. Additionally, compounds active against fungal prenyltransferases, e.g., GGPTase I, may be screened independently against mammalian prenyltransferases. The present invention is not limited in terms of the methods relied upon for pinpointing potent inhibitors. Compounds selected based on their activity in vitro may be screened subsequently in vivo.

In one embodiment, a candidate inhibitor can be tested in an assay comprising a prenylation reaction system that includes a prenyltransferase (e.g., FPTase, GGPTase I, and GGPTase II) or N-myristoyltransferase; a suitable protein for prenylation or myristoylation by the particular transferase of the assay, or a portion thereof, which serves as a target substrate; and an activated moiety to serve as the isoprenoid or myristoyl donor which can be covalently attached to the substrate by the transferase. The level of prenylation/myristoylation of the target substrate brought about by the system is measured in the presence and absence of a candidate agent, and a statistically significant decrease in the level prenylation/myristoylation is indicative of a potential activity for the candidate agent of interest. In one embodiment, the transferase is a fungal GGPTase, the suitable target is a fungal GTPase protein or portion thereof, and the activated moiety is a geranylgeranyl moiety. In other preferred embodiments, the assay system is designed for use with fungal N-myristoyltransferase or fungal FTPase.

As described below, the level of prenylation/myristoylation of the target protein can be measured by determining the actual concentration of substrate: conjugates formed; or inferred by detecting some other quality of the target substrate affected by prenylation/myristoylation, including membrane localization of the target. In certain embodiments, the present assay comprises an in vivo prenylation/myristoylation system, such as a cell able to conduct the target substrate through at least a portion of a conjugation pathway. In other embodiments, the present assay comprises an in vitro prenylation/myristoylation system in which at least the ability to transfer isoprenoids/myristoyl groups to the target protein is constituted. Still other embodiments provide assay formats which detect protein-protein interaction between the prenyltransferase/N-myristoyltransferase and a target protein, rather than enzymatic activity per se.

Analogous assays for measuring inhibition of CAK1 will be apparent to those of ordinary skill in the art.

Cell-Free Assay Formats

In many drug-screening programs that test libraries of compounds and natural extracts, high-throughput assays are desirable in order to maximize the number of compounds surveyed in a given period of time. Assays which are performed in cell-free systems, such as may be derived with purified or semi-purified proteins or cell-lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, a reaction mixture is generated to include a polypeptide for prenylation, such as Ras or other protein having GTPase-activity, candidate inhibitor(s) of interest, and a polypeptide having prenylation activity, such as FPTase, GGPTase I, or GGPTase II or a portion thereof retaining enzymatic activity. Detection and quantification of the enzymatic conversion of the polypeptide for prenylation or the formation of complexes containing the polypeptide for prenylation and the polypeptide having prenylation activity provide a means for determining a compound's efficacy at inhibiting (or potentiating) the complex bioactivity of any prenyltransferase. The efficacy of the compound can be assessed by generating dose response curves from data obtained using various concentrations of the test compound. Moreover, a control assay may also be performed to provide a baseline for comparison.

In one embodiment, the subject drug screening assay comprises a prenylation/myristoylation system, e.g., a reaction mixture which enzymatically conjugates isoprenoids/myristoyl groups to a target protein, which is arranged to detect inhibitors of the prenylation of a Rho-like GTPase or the myristoylation of an N-myristoyltransferase. For instance, in one embodiment of a cell-free prenylation system, one or more cell lysates including a prenyltransferase, a Rho-like GTPase (or substrate analog thereof), and an activated isoprenoid group are incubated with the test compound and the level of prenylation of the Rho-like GTPase substrate is detected. Lysates can be derived from cells expressing one or more of the relevant proteins, and mixed appropriately (or split) where no single lysate contains all the components necessary for generating the prenylation system. In preferred embodiments, one or more of the components, especially the substrate target, are recombinantly produced in a cell used to generate a lysate, or added by spiking a lysate mixture with a purified or semi-purified preparation of the substrate. These embodiments have several advantages including: the ability to use a labeled substrate, e.g., a dansylated peptide, or fusion protein, e.g., a Rho1-GST fusion protein, for facilitating purification; the ability to carefully control reaction conditions with respect to concentrations of reactants; and where targets are derived from fungal pathogens, the ability to work in a non-pathogenic system by recombinantly or synthetically by producing components from the pathogen for constituting the prenylation system. In other preferred embodiments, the transferase is fungal N-myristoyltransferase, FTPase, GGPTase I, or GGPTase II. In certain preferred embodiments, the transferase is fungal GGPTase.

The assay systems can be derived from any number of cell types, ranging from bacterial cells to yeast cells to cells from metazoan organisms including insects and mammalian cells. To illustrate, a fungal prenylation system can be reconstituted by mixing cell lysates derived from insect cells expressing prenyltransferase subunits cloned into baculoviral expression vectors. For example, the exemplary GGPTase-I expression vectors described below can be recloned into baculoviral vectors (e.g., pVL vectors), and recombinant GGPTase-I produced in transfected Spodoptera fungiperda cells. The level of activity can be assessed by enzymatic activity, or by quantitating the level of expression by detecting, e.g., an exogenous tag added to the recombinant protein. Substrate and activated geranylgeranyl diphosphate can be added to the lysate mixtures. As appropriate, the transfected cells can be cells which lack an endogenous GGPTase activity, or the substrate can be chosen to be particularly sensitive to prenylation by the exogenous fungal GGPTase relative to any endogenous activity of the cells. In other embodiments, other prenyltransferases are employed.

In other cell-free embodiments of the present assay, the assay system comprises a reconstituted protein mixture of at least semi-purified proteins. By semi-purified, it is meant that the proteins utilized in the reconstituted mixture have been previously separated from other cellular proteins. For instance, in contrast to cell lysates, the transferase proteins involved in conjugation of moieties to a target protein, together with the target protein, are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins which might interfere with or otherwise alter the ability to measure specific prenylation/myristoylation rates of the target substrate.

In the subject method, prenylation/myristoylation systems derived from purified proteins may have certain advantages over cell lysate based assays. Unlike the reconstituted protein system, the prenylation/myristoylation activity of a cell-lysate may not be readily controlled. Measuring kinetic parameters is made tedious by the fact that cell lysates may be inconsistent from batch to batch, with potentially significant variation between preparations. For example, in vitro evidence indicates that prenyltransferases have the ability to cross-prenylate CAAX-related sequences, so that prenyltransferase not of interest present in a lysate may provide an unwanted kinetic parameter. Moreover, cycling of prenylated proteins by guanine nucleotide dissociation inhibitor (GDI)-like proteins in the lysate could further complicate kinetics of the reaction mixture. Evaluation of a potential inhibitor using a lysate system is also complicated in those circumstances where the lysate is charged with mRNA encoding the a substrate polypeptide, e.g., GTPase, or prenyltransferase activity, as such lysates may continue to synthesize proteins active in the assay during the development period of the assay, and can do so at unpredictable rates. Knowledge of the concentration of each component of the prenylation/myristoylation system can be required for each lysate batch, along with the overall kinetic data, in order to determine the necessary time course and calculate the sensitivity of experiments performed from one lysate preparation to the next. The use of reconstituted protein mixtures can allow more careful control of the reaction conditions in the prenylation/myristoylation reaction.

The purified protein mixture includes a purified preparation of the substrate polypeptide and an isoprenoid moiety or N-myristoyl source (or analog thereof) under conditions which drive the conjugation of the two molecules. For instance, the mixture can include a fungal GGPTase I complex including RAM2 and CDC43 subunits, a geranylgeranyl diphosphate, a divalent cation, and a substrate polypeptide, such as may be derived from Rho1.

Prenylation/myristoylation of the target regulatory protein via an in vitro prenylation system, in the presence and absence of a candidate inhibitor, can be accomplished in any vessel suitable for containing the reactants. Examples include microtitre plates, test tubes, and micro-centrifuge tubes. In such embodiments, a wide range of detection means can be practiced to score for the presence of the prenylation/myristoylation protein.

In one embodiment of the present assay, the products of a prenylation/myristoylation system are separated by gel electrophoresis, and the level of prenylated/myristoylated substrate polypeptide assessed, using standard electrophoresis protocols, by measuring an increase in molecular weight of the target substrate that corresponds to the addition of one or more such moieties. For example, one or both of the target substrate and transferred group can be labeled with a radioisotope such as ³⁵S, ¹⁴C, or ³H, and the isotopically labeled protein bands quantified by autoradiographic techniques. Standardization of the assay samples can be accomplished, for instance, by adding known quantities of labeled proteins that are not themselves subject to prenylation/myristoylation or degradation under the conditions which the assay is performed. Similarly, other means of detecting electrophoretically separated proteins can be employed to quantify the level of prenylation of the target substrate, including immunoblot analysis using antibodies specific for either the target substrate or isoprenoid/myristoyl epitopes.

As described below, the antibody can be replaced with another molecule able to bind one of either the target substrate or the isoprenoid/myristoyl group. By way of illustration, one embodiment of the present assay comprises the use of a biotinylated target substrate in the conjugating system. For example, biotinylated GGPTase substrates have been described in the art (c.f. Yokoyama et al. (1995) Biochemistry 34:1344-1354). The biotin label is detected in a gel during a subsequent detection step by contacting the electrophoretic products (or a blot thereof) with a streptavidin-conjugated label, such as a streptavidin linked fluorochrome or enzyme, which can be readily detected by conventional techniques. Moreover, where a reconstituted protein mixture is used (rather than a lysate) as the conjugating system, it may be possible to simply detect the target substrate and isoprenoid/myristoyl conjugates in the gel by standard staining protocols, including coomassie blue and silver staining.

In a similar fashion, altered and unaltered substrate can be separated by other chromatographic techniques, and the relative quantities of each determined. For example, HPLC can be used to quantitate prenylated and unprenylated substrate (Pickett et al. (1995) Analytical Biochem 225:60-63), and the effect of a test compound on that ratio determined.

In another embodiment, an immunoassay or similar binding assay, is used to detect and quantify the level of prenylated/myristoylated target substrate produced in the prenylation/myristoylation system. Many different immunoassay techniques are amenable for such use and can be employed to detect and quantitate the conjugates. For example, the wells of a microtitre plate (or other suitable solid phase) can be coated with an antibody which specifically binds one of either the target substrate or isoprenoid/myristoyl groups. After incubation of the prenylation/myristoylation system with and without the candidate agent, the products are contacted with the matrix bound antibody, unbound material removed by washing, and altered conjugates of the target substrate specifically detected. To illustrate, if an antibody which binds the target substrate is used to sequester the protein on the matrix, then a detectable anti-isoprenoid/myristoyl antibody can be used to score for the presence of conjugated target substrate on the matrix.

Still a variety of other formats exist which are amenable to high throughput analysis on microtitre plates or the like. The prenylation/myristoylation substrate can be immobilized throughout the reaction, such as by cross-linking to activated polymer, or sequestered to the well walls after the development of the prenylation/myristoylation reaction. In one illustrative embodiment, a Rho-like GTPase, e.g., a fungal Rho1, Rho2, Cdc42 or Rsr1/Bud1, is cross-linked to the polymeric support of the well, the prenylation system set up in that well, and after completion, the well washed and the amount of geranylgeranyl sidechains attached to the immobilized GTPase detected. In another illustrative embodiment, wells of a microtitre plate are coated with streptavidin and contacted with a developed prenylation/myristoylation system under conditions wherein a biotinylated substrate binds to and is sequestered in the wells. Unbound material is washed from the wells, and the level of conjugated target substrate is detected in each well. There are, as evidenced by this specification, a variety of techniques for detecting the level of prenylation/myristoylation of the immobilized substrate. For example, by the use of dansylated (described infra) or radiolabelled isoprenoid/myristoyl moieties in the reaction mixture, addition of appropriate scintillant to the wells will permit detection of the label directly in the microtitre wells. Alternatively, the substrate can be released and detected, for example, by any of those means described above, e.g., by radiolabel, gel electrophoresis, etc. Reversibly bound substrate, such as the biotin-conjugated substrate set out above, is particularly amenable to the latter approach. In other embodiments, only the isoprenoid/myristoyl moiety is released for detection. For instance, the thioether linkage of the isoprenoid with the substrate peptide sequence can be cleaved by treatment with methyl iodide. The released isoprenoid/myristoyl products can be detected, e.g., by radioactivity, HPLC, or other convenient format.

Other isoprenoid/myristoyl derivatives include detectable labels which do not interfere greatly with the conjugation of that group to the target substrate. For example, in an illustrative embodiment, the assay format provides fluorescence assay which relies on a change in fluorescent activity of a group associated with a transferase substrate to assess test compounds against a transferase. To illustrate, prenylation activity of any prenyltransferase may be measured by a modified version of the continuous fluorescence assay described for farnesyl transferases (Cassidy et al., (1985) Methods Enzymol. 250: 30-43; Pickett et al. (1995) Analytical Biochem 225:60-63; and Stirtan et al. (1995) Arch Biochem Biophys 321:182-190). In an illustrative embodiment, dansyl-Gly-Cys-Ile-Ile-Leu (d-GCIIL) and geranylgeranyl diphosphate are added to assay buffer, along with the test agent or control. This mixture is preincubated at 30° C. for a few minutes before the reaction is initiated with the addition of GGPTase enzyme. The sample is vigorously mixed, and an aliquot of the reaction mixture immediately transferred to a prewarmed cuvette, and the fluorescence intensity measured for 5 minutes. Useful excitation and emission wavelengths are 340 and 486 nm, respectively, with a bandpass of 5.1 nm for both excitation and emission monochromators. Generally, fluorescence data are collected with a selected time increment, and the inhibitory activity of the test agent is determined by detecting a decrease in the initial velocity of the reaction relative to samples that lack a test agent.

In yet another embodiment, the transferase activity against a particular substrate can be detected in the subject assay by using a phosphocellulose paper absorption system (Roskoski et al. (1994) Analytical Biochem 222:275-280), or the like. To effect binding of a peptidyl substrate to phosphocellulose at low pH, several basic residues can be added, preferably to the amino-terminal side of the target sequence of the peptide, to produce a peptide with a minimal minimum charge of +2 or +3 at pH less than 2. This follows the strategy used for the phosphocellulose absorption assay for protein kinases. In one embodiment; the transfer of a [H³] isoprenoid group from [H³]-isoprenoid pyrophosphate to acceptor peptides can be measured under conditions similar to the farnesyl transferase reactions described by Reiss et al. (Reiss et al., (1990) Cell 62: 81-88). In an illustrative embodiment, the transfer of the [H³] geranylgeranyl group from [H³]-geranylgeranyl pyrophosphate to KLKCAIL can be measured. Reaction mixtures can be generated to contain 50 mM Tris-HCL (pH 7.5), 50 μM ZnCl₂, 20 mM KCl, 1 mM dithiothreitol, 250 μM KLKCAIL, 0.4 μM [H³] geranylgeranyl pyrophosphate, and 10-1000 μg/ml of purified fungal GGPTase protein. After incubation, e.g., for 30 minutes at 37° C., samples are applied to Whatman P81 phosphocellulose paper strips. After the liquid permeates the paper (a few seconds), the strips are washed in ethanol/phosphoric acid (prepared by mixing equal volumes of 95% ethanol and 75 mM phosphoric acid) to remove unbound isoprenoids. The samples are air dried, and radioactivity can be measured by liquid scintillation spectrometry. Background values are obtained by using reaction mixture with buffer in place of enzyme.

An added feature of this strategy is that it produces hydrophilic peptides that are more readily dissolved in water. Moreover, the procedure outlined above works equally well for protein substrates (most proteins bind to phosphocellulose at acidic pH), so should be useful where full length protein, e.g., Rho1 or Cdc42, are utilized as the prenylation substrate, e.g., substrate for GGPTase.

The ability of a test compound to inhibit N-myristoyltransferase can also be readily measured using assays such as those disclosed in Devadas et al., J Med Chem 1997, 40, 2609-25; Lodge et al., Microbiology 1997, 143, 357-66; and Zheng et al., J Pharm Sci 1994, 83, 233-8, for example, as the assays described above.

To test potential inhibitors of CAK1, a reaction mixture is generated to include an CAK1 polypeptide, compound(s) of interest, and one or more “target polypeptides”, e.g., proteins, which interacts with the CAK1 polypeptide. Exemplary target polypeptides include cyclin dependent kinases (such as cdc28, CDK1 and Kin28) and CAK activating kinases. Detection and quantification of the formation of complexes including the CAK1 protein provides a means for determining a compound's efficacy at inhibiting (or potentiating) the bioactivity of CAK1. “Interaction” may be manifested as as phosphorylation of a CAK1 substrate, or phosphorylation of CAK1 as the substrate. The efficacy of a test compound can be assessed by generating dose response curves from data obtained using various concentrations of the test compound. Moreover, a control assay can also be performed to provide a baseline for comparison.

In certain instances, a drug-screening assay comprises a reconstituted protein mixture of at least semi-purified proteins. By semi-purified, it is meant that the proteins utilized in the reconstituted mixture have been previously separated from other cellular proteins. For instance, in contrast to cell lysates, the CAK1 protein and substrate (or other associated proteins) are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins that might interfere with or otherwise alter the ability to measure specific phosphorylation rates of the target CAK1 substrate.

Complex formation between the CAK1 polypeptide and a arget polypeptide (e.g., a protein or protein complex which binds to the CAK1 polypeptide) may be detected by a variety of techniques. Modulation of the formation of complexes can be quantitated using, for example, detectably labeled proteins such as radiolabelled (e.g., ³²P, ³⁵S, ¹⁴C or ³H), fluorescently labeled (e.g., FITC), or enzymatically labeled CAK1 polypeptides, by immunoassay, by chromatographic detection, or preferably, by detecting the intrinsic activity of either the CAK1 or target polypeptide. The use of enzymatically labeled proteins will, of course, generally be used only when enzymatically inactive portions of those proteins are used, as such target proteins as Cdk1 or Kin28 can possess a measurable intrinsic activity which can be detected, e.g., by phosphorylation of a histone protein with a labeled phosphate group.

Typically, it will be desirable to immobilize either the CAK1 or the target polypeptide to facilitate separation of complexes from uncomplexed forms, or free enzyme from substrate, as well as to accommodate automation of the assay. Binding of a CAK1 polypeptide to the target polypeptide, or phosphorylation reactions, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/CAK1 (GST/CAK1) fusion proteins can be adsorbed onto glutathione sepharose beads (Sigma Chemical, St. Louis, Mo.) or glutathione-derivatized microtitre plates, which are then combined with a preparation of a target polypeptide, e.g., a labeled target polypeptide, along with the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and labeled target polypeptide retained on the matrix determined directly, or in the supernatant after the complexes are subsequently dissociated. Alternatively, the complexes can be dissociated from the matrix, separated by SDS-PAGE, and the level of target polypeptide found in the bead fraction quantitated from the gel using standard electrophoretic techniques.

Other techniques for immobilizing proteins on matrices are also available for use in the subject assay. For instance, either the CAK1 or target polypeptide can be immobilized utilizing conjugation of biotin and streptavidin. For instance, biotinylated CAK1 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with CAK1, but which do not interfere with the interaction between the CAK1 and target polypeptide, can be derivatized to the wells of the plate, and CAK1 trapped in the wells by antibody conjugation. As above, preparations of a target polypeptide and a test compound are incubated in the CAK1-presenting wells of the plate, and the amount of complex trapped in the well can be quantitated. Other exemplary methods for detecting such complexes, in addition to those described above, include detection of a radiolabel or fluorescent label; immunodetection of complexes using antibodies reactive with the target polypeptide, or which are reactive with CAK1 protein and compete with the target polypeptide; as well as enzyme-linked assays which rely on detecting an enzymatic activity associated with the target polypeptide, e.g., either intrinsic or extrinsic activity.

In the instance of the latter, the enzyme can be chemically conjugated or provided as a fusion protein with the target polypeptide. To illustrate, the target polypeptide can be chemically cross-linked or genetically fused with horseradish peroxidase, and the amount of polypeptide trapped in the complex can be assessed with a chromogenic substrate of the enzyme, e.g. 3,3′-diamino-benzadine terahydrochloride or 4-chloro-1-napthol. Likewise, a fusion protein comprising the target polypeptide and glutathione-S-transferase can be provided, and complex formation quantitated by detecting the GST activity using 1-chloro-2,4-dinitrobenzene (Habig et al (1974) J Biol Chem 249:7130).

As alluded to above, intrinsic enzymatic activities can be relied upon to detect the efficacy of an agent against CAK1. The detection of the CAK1 kinase activity is described in more detail below. However, the downstream targets of CAK1, such as a CDK, may also have an intrinsic activity that can be utilized to quantitate the interaction with CAK1. In an exemplary embodiment, an enzymatically active CAK1 is contacted with a phosphorylated CDK/cyclin complex, e.g. CDK1/CYB1, under conditions wherein, absent an inhibitor of the CAK1, that enzyme would phosphorylate and activate the CDK/cyclin complex. Activation could be detected by conversion of a substrate for the kinase complex, such as phosphorylation of a histone H1 protein with ³²P-labeled phosphate.

For processes that rely on immunodetection for quantitating one of the proteins trapped in the complex, antibodies against the target protein or CAK1 protein, can be used. Alternatively, the protein to be detected in the complex can be “epitope tagged” in the form of a fusion protein that includes a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) that include a 10-residue sequence from c-myc, as well as the pFLAG system (International Biotechnologies, Inc.) or the pEZZ-protein a system (Pharamacia, N.J.).

In other embodiments, the cell-free mixtures can be generated using lysates, e.g., derived from cells expressing one or more of the relevant proteins, and mixed appropriately (or spiked) where no single lysate contains all the components necessary for generating the reaction system. In preferred embodiments, one or more of the components, especially the substrate target, are recombinantly produced in a cell used to generate a lysate, or added by spiking a lysate mixture with a purified or semi-purified preparation of the substrate.

The lysates can be derived from any number of cell types, ranging from bacterial cells to yeast cells to cells from metazoan organisms including insects and mammalian cells. To illustrate, a cell-free test system can be reconstituted by mixing cell lysates derived from insect cells expressing CAK1 and the target protein which have been cloned into baculoviral expression vectors. The cells can be lysed, and if the CAK1 and target protein are produced by different sets of cells, cell lysates can be accordingly mixed to produce CAK1 complexes. The level of protein-protein interaction, or if applicable, the enzymatic activity of the complex, can be assessed. As appropriate, the transfected cells can be cells which lack an endogenous CAK1 protein, or the target protein, can be chosen to be particularly sensitive to avoiding endogenous activity of the cells which may confound the results.

Moreover, for each of the subject regulatory proteins which have intrinsic enzymatic activities, such as the CDC25, CAK1, CDK1, KIN28 and CMK1 proteins, the present invention provides methods and reagents for identifying agents which inhibit the enzymatic activity of the protein, e.g., agents which are mechanism based inhibitors of the enzyme, rather than merely disrupting the formation of a protein complex. Inhibitors of the enzymatic activity can be identified, for example, using assays generated for measuring the ability of an agent to inhibit catalytic conversion of a substrate by one of the subject enzymes. Again using CAK1 as an illustrative embodiment, a molecule or compound (e.g. a “test agent”) to be assessed for its ability to inhibit the kinase activity of the subject CAK1 enzyme is combined with the enzyme and a substrate of its kinase activity. The resulting combination is maintained under conditions appropriate for the CAK1 enzyme to act upon the substrate. For instance, the reaction mixture will include ATP, or an analog thereof, appropriate salts, buffers, etc. The conversion of the substrate to product by the enzyme is assessed, and the result compared to the rate or level of conversion of the substrate in the absence of the test agent. A statistically significant decrease in the activity of the CAK1 kinase in the presence of the test agent, manifest as a decrease in conversion of substrate to product, indicates that the test agent is an inhibitor of the kinase.

For example, the substrate can be a cyclin-dependent kinase, such as cdc28, CDK1, Kin28, or Pho85 or Slb10, from a fungal cell (preferably Candida), or a mammalian cdk such as cdk2 or cdk6, or peptide fragments thereof which retain the Thr-169 residue (e.g., Thr166 of CDK1 or Thr200 of KIN28). The reaction mixture may also include cyclins and other regulatory proteins which associate with the substrate. For instance, where the substrate is KIN28, the reaction mixture may also include the KIN28 regulatory subunits Ccl1 and Tfb3. In preferred embodiments, the cdk is dephosphorylated at Thr169. In preferred embodiments, the cdk is phosphorylated at Thr14 and Tyr 15. Phosphorylation of such substrates by CAK1 can be detected by incorporation of a labeled phosphate group, or by measuring activation of the cdk, e.g., as a kinase.

In preferred embodiments, the substrate of CAK1 is a synthetic substrate, e.g., a peptide or tyrosine analog, comprising a colorimetric or fluorescent label which is detectable when the substrate is catalytically acted upon by the kinase. As used herein “colorimetric” refers to substrates detectable by change in absorption or fluorescent characteristics. Yet other substrates include peptides that can be radiolabeled, e.g. using ³²P-labeled ATP, wherein an increase in the radiolabeling of the peptide can be detected and correlated with CAK1 enzymatic activity.

In an illustrative embodiment, the method comprises the steps of: (a) combining a compound to be assessed, the subject Candida CAK1 (purified or semipurified), and a substrate of the pathogen CAK1 kinase activity comprising a label which undergoes a detectable change when the substrate is acted upon by the kinase; (b) maintaining the substrate/enzyme/test compound combination under conditions appropriate for the pathogen-derived CAK1 to act upon the substrate; and (c) determining, by defections of the label, the extent to which the CAK1 enzyme present in the combination acted upon the substrate, relative to a control, the control comprising the CAK1 enzyme and the substrate without the test compound. If the subject CAK1 enzyme acts upon the substrate to a lesser extent than in the control, the compound is an inhibitor of the CAK1 kinase activity.

In still other assays, the CAK1 protein, or a suitable portion thereof, is provided in the reaction mixture along with a CAK activating kinase, such as Csk1 (see Hermand et al. (1998) EMBO J 17:7230). In the absense of an inhibitor of the CAKAK, the CAK1 polypeptide and CAKAK will interact, and preferably, the CAK1 polypeptide will be phosphorylated. Inhibitors of that interaction and/or the enzymatic activity of the CAKAK can be identified.

Cell-Based Assay Formats

In other embodiments, compounds for use in the subject method can be tested using a screening assay derived to include a whole cell expressing a substrate protein, along with a prenyltransferase (e.g., FTPase, GGPTase I, and GGPTase II). CAK1, or N-myristoyltransferase. In preferred embodiments, the reagent cell is a mammalian cell that has been engineered to express one or more of these proteins from mammalian recombinant genes. In other preferred embodiments, the reagent cell is a fungal cell that has been engineered to express one or more of these proteins from fungal recombinant genes. The reagent cell may be manipulated so that the recombinant gene(s) complement a loss-of-function mutation to the homologous gene in the reagent cell.

In preferred embodiments, the reagent cell is a non-pathogenic cell which has been engineered to express one or more of these proteins from recombinant genes cloned from a pathogenic fungus. For example, non-pathogenic fungal cells, such as S. cerevisae, can be derived to express a Rho-like GTPase from a fungal pathogen such as Candida albicans. In an exemplary embodiment, a non-pathogenic yeast cell is engineered to express a Rho-like GTPase, e.g., Rho1, and at least one of the subunits of a GGPTase, e.g., RAM2 and/or Cdc43, derived from a fungal protein. One salient feature to such reagent cells is the ability of the practitioner to work with a non-pathogenic strain rather than the pathogen itself. Another advantage derives from the level of knowledge, and available strains, when working with such reagent cells as S. cerevisae.

In other embodiments, compounds for use in the subject method can be detected using a screening assay derived to include a whole cell expressing a substrate for a prenyltransferase, e.g., a GTPase protein, CAK1, e.g., CAKAK, or N-myristoyltransferase, along with the appropriate transferase. In preferred emboidments, the reagent cell is a mammalian cell that has been engineered to express one or more of these proteins from recombinant mammalian genes. In other preferred embodiments, the reagent cell is a non-fungal cell that has been engineered to express one or more of these proteins from recombinant mammalian genes. In other preferred embodiments, the reagent cell is a non-pathogenic cell that has been engineered to express one or more of these proteins from recombinant genes cloned from a pathogenic fungus. For example, non-pathogenic fungal cells, such as S. cerevisae, can be derived to express a Rho-like GTPase from a fungal pathogen such as Candida albicans. In an exemplary embodiment, a non-pathogenic yeast cell is engineered to express a Rho-like GTPase, e.g., Rho1, and at least one of the subunits of a GGPTase, e.g., RAM2 and/or Cdc43, derived from a fungal protein. One salient feature to such reagent cells is the ability of the practitioner to work with a non-pathogenic strain rather than the pathogen itself Another advantage derives from the level of knowledge, and available strains, when working with such reagent cells as S. cerevisae. For all such embodiments, the reagent cell may be manipulated such that the recombinant gene(s) complement a loss-of-function mutation to the homologous gene in the reagent cell.

The ability of a test agent to alter the activity of a prenyltransferase or N-myristoyltransferase may be detected by analysis of the cell or products produced by the cell. For example, inhibitors of N-myristoyltransferase, prenyltransferase, or CAK1 can be detected by scoring for alterations in growth or viability of the cell. Other embodiments will permit inference of the level of activity based on, for example, detecting expression of a reporter, the induction of which is directly or indirectly dependent on the activity of an enzyme substrate. General techniques for detecting each are well known, and will vary with respect to the source of the particular reagent cell utilized in any given assay.

Quantification of proliferation of cells in the presence and absence of a candidate agent can be measured with a number of techniques well known in the art, including simple measurement of population growth curves. For instance, where the assay involves proliferation in a liquid medium, turbidimetric techniques (i.e., absorption/transmission of light of a given wavelength through the sample) can be utilized. For example, in the instance where the reagent cell is a yeast cell, measurement of absorption of light at a wavelength between 540 and 600 nm can provide a conveniently fast measure of cell growth. Likewise, ability to form colonies in solid medium (e.g., agar) can be used to readily score for proliferation. In other embodiments, a GTPase substrate protein, such as a histone, can be provided as a fusion protein that permits the substrate to be isolated from cell lysates and the degree of acetylation detected. Each of these techniques is suitable for high through-put analysis necessary for rapid screening of large numbers of candidate agents.

Additionally, visual inspection of the morphology of the reagent cell can be used to determine whether the biological activity of the targeted protein, e.g., GTPase, has been affected by the added agent. To illustrate, the ability of an agent to create a lytic phenotype which is mediated in some way by a recombinant GTPase protein can be assessed by visual microscopy.

The nature of the effect of test agent on reagent cell can be assessed by measuring levels of expression of specific genes, e.g., by reverse transcription-PCR. Another method of scoring for effect on protein activity of interest, e.g., GTPase, is by detecting cell-type specific marker expression through immunofluorescent staining. Many such markers are known in the art, and antibodies are readily available.

In yet another embodiment, in order to enhance detection of cell lysis for fungal inhibitors, the target cell can be provided with a cytoplasmic reporter which is readily detectable, either because it has “leaked” outside the cell, or substrate has “leaked” into the cell, by perturbations in the cell wall. Preferred reporters are proteins which can be recombinantly expressed by the target cell, do not interfere with cell wall integrity, and which have an enzymatic activity for which chromogenic or fluorogenic substrates are available. In one example, a fungal cell can be constructed to recombinantly express the β-galactosidase gene from a construct (optionally) including an inducible promoter. At some time prior to contacting the cell with a test agent, expression of the reporter protein is induced. Agents which inhibit, for example, prenylation of a Rho-like GTPase in the cell, or the subsequent involvement of a Rho-like GTPase in cell wall integrity, can be detected by an increase in the reporter protein activity in the culture supernatant or from permeation of a substrate in the cell. Thus, for example, β-galactosidase activity can be scored using such colorimetric substrates as 5-bromo-4-chloro-3-indolyl-β-D-galactopyranoside or fluorescent substrates such as methylumbelliferyl-β-D-galactopyranoside. Permeation of the substrate into the cell, or leakage of the reporter into the culture media, is thus readily detectable.

In still another embodiment, the membrane localization resulting from prenylation of a GTPase can be exploited to generate the cell-based assay. For instance, the subject assay can be derived with a reagent cell having: (i) a reporter gene construct including a transcriptional regulatory element which can induce expression of the reporter upon interaction of the transcriptional regulatory protein portion of the above fusion protein. For example, a ga14 protein can be fused with a Rho1 polypeptide sequence which includes the CAAX prenylation target. In the absence of inhibitors of GGPTase activity in the reagent cell, prenylation of the fusion protein will result in partitioning of the fusion protein at the cell surface membrane. This provides a basal level of expression of the reporter gene construct. When contacted with an agent that inhibits prenylation of the fusion protein, partitioning is lost and, with the concomitant increase in nuclear concentration of the protein, expression from the reporter construct is increased.

In a preferred embodiment, the cell is engineered such that inhibition by fungal inhibitors of the GGPTase activity does not result in cell lysis. For example, as described in Ohya et al. (1993) Mol Cell Biol 4:1017-1025, mutation of the C-terminus of Rho1 and cdc42 can provide proteins which are targets of farsenyl transferase rather than geranylgeranyl transferase. As Ohya et al. describe, such mutants can be used to render the GGPTase I activity dispensable. Accordingly, providing a reporter gene construct and an expression vector for the GGPTase substrate/transcription factor fusion protein in such cells as YOT35953 cells (Ohya et al., vide supra) generates a cell whose viability vis-a-vis the GGPTase activity is determined by the reporter construct, if at all, rather than by prenylation of an endogenous Rho-like GTPase by the GGPTase. Of course, the reporter gene product can be derived to have no effect on cell viability, providing for example another type of detectable marker (described, infra). Such cells can be engineered to express an exogenous GGPTase activity in place of an endogenous activity, or can rely on the endogenous activity. To further illustrate, the Call mutant YOT35953 cell can be further manipulated to express a Call homolog from, e.g., a fungal pathogen or a mammalian cell.

Alternatively, where fungal inhibition of an enzymatic activity causes cell lysis and reporter gene expression, the leakage assay provided above can be utilized to detect expression of the reporter protein. For instance, the reporter gene may encode β-galactosidase, and inhibition of the enzyme's activity scored for by the presence of cells which take up substrate due to loss of cell wall integrity, and convert substrate due to the expression of the reporter gene.

In other preferred embodiments, the reporter gene is a gene whose expression causes a phenotypic change which is screenable or selectable. If the change is selectable, the phenotypic change creates a difference in the growth or survival rate between cells which express the reporter gene and those which do not. If the change is screenable, the phenotype change creates a difference in some detectable characteristic of the cells, by which the cells which express the marker may be distinguished from those which do not.

The marker gene is coupled to enzyme-dependent activity, be it membrane association, or a downstream signaling pathway induced by an enzyme complex, so that expression of the marker gene is dependent on the activity of the enzyme. This coupling may be achieved by operably linking the marker gene to a promoter responsive to the therapeutically targeted event. The term “enzyme-responsive promoter” indicates a promoter which is regulated by some product or activity of the fungal enzyme. By this manner, for example, the activity of a prenyltransferase may be detected by its effects on prenylation of GTPase and, accordingly, the downstream targets of the prenylated protein. Thus, in certain embodiments, transcriptional regulatory sequences responsive to signals generated by PKC/GTPase, GS/GTPase and/or other GTPase complexes, or to signals by other proteins in such complexes which are interrupted by GTPase binding, can be used to detect function of Rho-like GTPases such as Rho1 and cdc42.

In the case of nonfungal systems, suitable positively selectable (beneficial) genes include the following: For yeast, suitable positively selectable (beneficial) genes include the following: URA3, LYS2, HIS3, LEU2, TRP1; ADE1, 2, 3, 4, 5, 7, 8; ARG1, 3, 4, 5, 6, 8; HIS1, 4, 5; ILV1, 2, 5; THR1, 4; TRP2, 3, 4, 5; LEU1, 4; MET2, 3, 4, 8, 9, 14, 16, 19; URA1, 2, 4, 5, 10; HOM3,6; ASP3; CHO]; ARO 2, 7, CYS3; OLE1; IN01, 2, 4; PRO1, 3. Countless other genes are potential selective markers. The above are involved in well-characterized biosynthetic pathways. The imidazoleglycerol phosphate dehydratase (IGP dehydratase) gene (HIS3) is preferred because it is both quite sensitive and can be selected over a broad range of expression levels. In the simplest case, the cell is auxotrophic for histidine (requires histidine for growth) in the absence of activation. Activation of the gene leads to synthesis of the enzyme and the cell becomes prototrophic for histidine (does not require histidine). Thus the selection is for growth in the absence of histidine. Since only a few molecules per cell of IGP dehydratase are required for histidine prototrophy, the assay is very sensitive.

The marker gene may also be a screenable gene. The screened characteristic may be a change in cell morphology, metabolism or other screenable features. Suitable markers include beta-galactosidase (Xgal, C₁₂FDG, Salmon-gal, Magenta-Gal (latter two from Biosynth Ag)), alkaline phosphatase, horseradish peroxidase, exo-glucanase (product of yeast exbl gene; nonessential, secreted); luciferase; bacterial green fluorescent protein; (human placental) secreted alkaline phosphatase (SEAP); and chloramphenicol transferase (CAT). Some of the above can be engineered so that they are secreted (although not β-galactosidase). A preferred screenable marker gene is β-galactosidase; for in yeast cells, for example, expression of the enzyme converts the colorless substrate Xgal into a blue pigment.

Moreover, the subject polypeptides can be used to generate an interaction trap assay, (see also, U.S. Pat. No. 5,283,317; Zervos et al. (1993) Cell 72:223-232; Madura et al. (1993) J Biol Chem 268:12046-12054; Bartel et al. (1993) Biotechnigues 14:920-924; and Iwabuchi et al. (1993) Oncogene 8:1693-1696), for subsequently detecting agents which disrupt binding of CAK1 to a CDK or CAKAK, a GGTPase to a GTPase, etc.

A similar method modifies the interaction trap system by providing a “relay gene” which is regulated by the transcriptional complex formed by the interacting bait and fish proteins. The gene product of the relay gene, in turn, regulates expression of a reporter gene, the expression of the latter being what is scored in the modified ITS assay. Fundamentally, the relay gene can be seen as a signal inverter. As set out above, in the standard ITS, interaction of the fish and bait fusion proteins results in expression of a reporter gene. However, where inhibitors of the interaction are sought, apositive readout from the reporter gene nevertheless requires detecting inhibition (or lack of expression) of the reporter gene.

In the inverted ITS system, the fish and bait proteins positively regulate expression of the relay gene. The relay gene product is in turn a repressor of expression of the reporter gene. Inhibition of expression of the relay gene product by inhibiting the interaction of the fish and bait proteins results in concomitant relief of the inhibition of the reporter gene, e.g., the reporter gene is expressed. For example, the relay gene can be the repressor gene under control of a promoter sensitive to the CAK1/CDK complex, GGTPase/GTPase, etc. The reporter gene can accordingly be a positive signal, such as providing for growth (e.g., drug selection or auxotrophic relief), and is under the control of a promoter which is constitutively active, but can be suppressed by the repressor protein. In the absence of an agent which inhibits the interaction of the fish and bait protein, the repressor protein is expressed. In turn, that protein represses expression of the reporter gene. However, an agent which disrupts binding of the CAK1, N-myristoyltransferase, or prenyltransferase and the target protein results in a decrease in repressor expression, and consequently an increase in expression of the reporter gene as repression is relieved. Hence, the signal is inverted.

In still other embodiments, the effect of a test compound on the virulence of a fungus can be assessed, e.g., in a mouse model of intravenous infection. Both adhesion and hyphal growth are hypothesized to be important for the pathogenicity of C. albicans. Returning to the teachings of Ohya et al. (1993), vide supra, it is noted that there are only two essential targets of GGPTase in S. cerevisae, the Rho-like GTPases Rho1 and cdc42. With such observations in mind, yet another embodiment of the subject assay utilizes a side-by-side comparison of the effect of a test agent on (i) a cell which prenylates a Rho-like GTPase by adding geranylgeranyl moieties, and (ii) a cell which prenylates an equivalent Rho-like GTPase by adding farnesyl moieties. In particular, the assay makes use of the ability to suppress GGPTase I defects in yeast by altering the C-terminal tail of Rho1 and cdc42 to become substrate targets of farnesyl transferase (see Ohya et al., supra). According to the present embodiment, the assay is arranged by providing a yeast cell in which the target Rho-like GTPases is prenylated by a GGPTase activity of the cell. Both the GGPTase and GTPase can be endogenous to the “test” cell, or one or both can be recombinantly expressed in the cell. The level of prenylation of the GTPase is detected, e.g., cell lysis or other means described above. The ability of the test compound to inhibit the addition of geranylgeranyl groups to the GTPase in the first cell is compared against the ability of test compound to inhibit the farnesylation of the GTPase in a control cell. The “control” cell is preferably identical to the test cell, with the exception that the targeted GTPase(s) are mutated at their CAAX sequence to become substrates for FPTases rather than GGPTases. Agents which inhibit prenylation in the test cell but not the control cell are selected as potential antifungal agents. Such differential screens can be exquisitely sensitive to inhibitors of GGPTase I prenylation of Rho-like GTPases. In a preferred embodiment, the test cell is derived from the S. cerivisae cell YOT35953 (Ohya et al., supra) or the like which is defective in GGPTase subunit cdc43. The cell is then engineered with a cdc43 subunit from a fungal pathogen such as Candida albicans to generate the test cell, and additionally with the mutated Rho-like GTPases to generate the control cell.

For other assays to measure inhibition of CAK1, for example, non-pathogenic fungal cells, such as S. cerevisae, can be derived to express a CAK1 protein from a fungal pathogen such as Candida albicans. Furthermore, the reagent cell can be manipulated, particularly if it is a yeast cell, such that the recombinant gene(s) complement a loss-of-function mutation to the homologous gene in the reagent cell. In an exemplary embodiment, a non-pathogenic yeast cell is engineered to express a CAK1 gene, e.g., a Candida enzyme CaCAK1. One salient feature to such reagent cells is the ability of the practitioner to work with a non-pathogenic strain rather than the pathogen itself. Another advantage derives from the level of knowledge, and available strains, when working with such reagent cells as S. cerevisae. Exemplary CAK1 loss-of-function strains are described by, e.g., Espinoza et al. (1998) Mol Cell Biol 18:8365.

The ability of a test agent to alter the activity of the CAK1 protein can be detected by analysis of the cell or products produced by the cell. For example, antagonists of CAK1-dependent growth, viability or pathogenecity can be detected by scoring for alterations in, e.g., hyphal growth or virulence of the cell. Other embodiments will permit inference of the level of CAK1 activity based on, for example, detecting expression of a reporter, the induction of which is directly or indirectly dependent on the activity of a CAK1 gene product. General techniques for detecting each are well known, and will vary with respect to the source of the particular reagent cell utilized in any given assay.

In one embodiment, the ability of the compound to inhibit CAK1-dependent adhesion and filamentous growth can be assessed. Such assays can be carried out on wild-type cells, e.g., to further screen compounds identified in the cell free assay, or as a primary screen, e.g., using a cell engineered for recombinant expression of CAK1 or other protein of an CAK1 complex. To illustrate, the specific adhesion of the test cell to, e.g., HeLa cells, can be assessed. To illustrate, ³⁵S-Methionine is added to exponentially growing yeast cells. Unlabeled yeast cells used to calculate nonspecific adhesion were grown identically. Yeast cells are harvested in midexponential phase, incubated for 1 hour at 37° C. with monolayers of human cervical carcinoma epithelial (HeLa) cells, and washed to remove nonadherent cells before release of the monolayer for scintillation counting. Specific adhesion is calculated as the difference between total adhesion [(cpm adherent cells/cpm total cells)×100] and nonspecific adhesion, the latter measured in the presence of a 100-fold excess of unlabeled yeast cells as described (Gustafson et al. (1991) J. Clin. Invest. 87:1896).

Scaringi et al. (1991) Mycoses 34(3-4):119 describe another in vitro microassay for the measurement of Candida albicans hyphal-form growth. The assay is rapid, easy-to-perform and objective. A Candida strain capable of in vitro dimorphic transition from yeast to hyphal form is employed. The assay is based on the incorporation of ³H-glucose by the fungus, the effect being dependent upon the time of pulse, size of the inoculum and concentration of radiolabelled metabolite.

Additionally, visual inspection of the morphology of the reagent cell can be used to determine whether CAK1-dependent growth, viability or pathogenecity has been affected by the added agent. To illustrate, the ability of an agent to create a lytic phenotype which is mediated in some way by a recombinant CAK1 protein can be assessed by visual microscopy.

For example, to quantify the fungistatic effect of a test compound identified in a cell-free assay, an cell-based assay can be used which based on the measurement is of hyphal growth of germinated spores. Hyphal formation is observed directly using an inverted tissue culture microscope, hyphal tips of higher fungi contain a characteristic phase-dark body: the Spitzenkorper (Spk), and the percentage of germination can be assessed with a hemacytometer.

Antihyphal activity can also be measured as percent inhibition of hyphal growth in assays using the dye MTT [3-(4,5-dimethylthiazol-2-yl)-2,5-diphenyltetrazolium bromide] or XTT [2,3-bis(2-methoxy-4-nitro-5-sulfophenyl)-2H-tetrazolium-5-carboxanilide]. Vora et al. (1998) Antimicrob Agents Chemother 42:2299

In yet another illustrative embodiment, the effect of a test compound on the filamentous growth of C. albicans strains can be monitored on two different media that induce filamentation, e.g., where the cells form an extensive network of long, branching hyphae that overlay and penetrated milk-Tween agar. Hyphal growth is induced in C. albicans strains by growth to stationary phase in SD minus uracil at 30° C., and then inoculation of the gungi on milk-Tween agar (Jitsurong et al. (1993) Mycopathologia 123:95) or on Spider medium with 1.35% agar, followed by incubation for 5 days at 30° and 37° C., respectively, to yield approximately 100 colonies per plate. The formation of wrinkled colonies on the plates is an indicator of filamentous growth. In contrast, formation of smooth-edged colonies with very few filamentous cells emanating from the colony edge indicates inhibition of hyphal growth.

The nature of the effect of test agent on reagent cell can also be assessed by measuring levels of expression of specific genes, e.g., by reverse transcription-PCR. Another method of scoring for effect on CAK1 activity is by detecting cell-type specific marker expression through immunofluorescent staining. Many such markers are known in the art, and antibodies are readily available.

The assays for growth inhibition of a microbial target can be used to derive an ED₅₀ value for the compound, that is, the concentration of compound required to kill 50% of the fungal sample being tested. Preferred antifuigal agent pharmaceutical preparation, whether for topical, injection or oral delivery (or other route of administration), would provide a dose less than the ED₅₀ for modulation of CAK1-, N-myristoyltransferase-, or GGTPase-dependent activity in the host (mammal), more preferably at least 1 order of magnitude less, more preferably at least 2, 3 or 4 orders of magnitude less.

Alternatively, growth inhibition by an antifungal compound of the invention may also be characterized in terms of the minimum inhibitory concentration (MIC), which is the concentration of compound required to achieve inhibition of fungal cell growth. Such values are well known to those in the art as representative of the effectiveness of a particular antifungal agent against a particular organism or group of organisms. For instance, cytolysis of a fungal population by an antifungal compound can also be characterized, as described above by the minimum inhibitory concentration, which is the concentration required to reduce the viable fungal population by 99.9%. The value of MIC₅₀ can also be used, defined as the concentration of a compound required to reduce the viable fungal population by 50%. In preferred embodiments, the compounds of the present invention are selected for use based, inter alia, on having MIC values of less than 25 μg/mL, more preferably less than 7 μg/mL, and even more preferably less than 1 μg/mL against a desired fungal target, e.g., Candida albicans.

Another parameter useful in identifying and measuring the effectiveness of the antifungal compounds of the invention is the determination of the kinetics of the antifungal activity of a compound. Such a determination can be made by determining antifungal activity as a function of time. In a preferred embodiment, the compounds display kinetics which result in efficient lysis of a fungal cell. In a preferred embodiment, the compounds are fungicidal.

Furthermore, the preferred antifungal compounds of the invention display selective toxicity to target microorganisms and minimal toxicity to mammalian cells. Determination of the toxic dose (or “LD₅₀”) can be carried out using protocols well known in the field of pharmacology. Ascertaining the effect of a compound of the invention on mammalian cells is preferably performed using tissue culture assays, e.g., the present compounds can be evaluated according to standard methods known to those skilled in that art (see for example Gootz, T. D. (1990) Clin. Microbiol. Rev. 3:13-31). For mammalian cells, such assay methods include, inter alia, trypan blue exclusion and MIT assays (Moore et al. (1994) Compound Research 7:265-269). Where a specific cell type may release a specific metabolite upon changes in membrane permeability, that specific metabolite may be assayed, e.g., the release of hemoglobin upon the lysis of red blood cells (Srinivas et al. (1992) J. Biol. Chem. 267:7121-7127). The compounds of the invention are preferably tested against primary cells, e.g., using human skin fibroblasts (HSF) or fetal equine kidney (FEK) cell cultures, or other primary cell cultures routinely used by those skilled in the art. Permanent cell lines may also be used, e.g., Jurkat cells. In preferred embodiments, the subject compounds are selected for use in animals, or animal cell/tissue culture based at least in part on having LD₅₀'s at least one order of magnitude greater than the MIC or ED₅₀ as the case may be, and even more preferably at least two, three and even four orders of magnitude greater. That is, in preferred embodiments where the subject compounds are to be administered to an animal, a suitable therapeutic index is preferably greater than 10, and more preferably greater than 100, 1000 or even 10,000.

Differential Screening Formats

In a preferred embodiment, assays can be used to identify compounds that have favorable therapeutic indexes. For instance, antifungal agents can be identified by the present assays which inhibit proliferation of yeast cells or other lower eukaryotes, but which have a substantially reduced effect on mammalian cells, thereby improving therapeutic index of the drug as an anti-mycotic agent.

In one embodiment, differential screening assays can be used to exploit the difference in protein interactions and/or catalytic mechanism of different transferases in order to identify agents which display a statistically significant increase in specificity for inhibiting certain prenylation/myristoylation reactions relative to others. Thus, lead compounds which act specifically on the certain prenylation/myristoylation reactions can be developed.

In another embodiment, differential screening assays can be used to exploit the difference in protein interactions and/or catalytic mechanism of mammalian and fungal CAK1, N-myristoyltransferase, or GGTPase enzymes in order to identify agents which display a statistically significant increase in specificity for inhibiting the fungal prenylation/myristoylation reaction relative to the mammalian prenylation/myristoylation reaction. Thus, lead compounds that act specifically on the prenylation/myristoylation reaction in pathogens, such as fungus involved in mycotic infections, can be developed. By way of illustration, the present assays can be used to screen for agents that may ultimately be useful for inhibiting the growth of at least one fungus implicated in such mycosis as candidiasis, aspergillosis, mucormycosis, blastomycosis, geotrichosis, cryptococcosis, chromoblastomycosis, coccidioidomycosis, conidiosporosis, histoplasmosis, maduromycosis, rhinosporidosis, nocaidiosis, para-actinomycosis, penicilliosis, monoliasis, or sporotrichosis. For example, if the mycotic infection to which treatment is desired is candidiasis, the present assay can comprise comparing the relative effectiveness of a test compound on inhibiting the prenylation of a mammalian GTPase protein with its effectiveness towards inhibiting the prenylation of a GTPase from a yeast selected from the group consisting of Candida albicans, Candida stellatoidea, Candida glabrata, Candida tropicalis, Candida parapsilosis, Candida krusei, Candida pseudotropicalis, Candida guilliermondii, or Candida rugosa. Likewise, the present assay can be used to identify antifungal agents which may have therapeutic value in the treatment of aspergillosis by selectively targeting, relative to human cells, GTPase homologs from yeast such as Aspergillus fumigatus, Aspergillus flavus, Aspergillus niger, Aspergillus nidulans, or Aspergillus terreus. Where the mycotic infection is mucormycosis, the GTPase system to be screened can be derived from yeast such as Rhizopus arrhizus, Rhizopus oryzae, Absidia corymbifera, Absidia ramosa, or Mucor pusillus. Sources of other assay reagents for includes the pathogen Pneumocystis carinii.

IV. Exemplification

The invention now being generally described, it will be more readily understood by reference to the following examples which are included merely for purposes of illustration of certain aspects and embodiments of the present invention, and are not intended to limit the invention.

Preparation of Compounds of the Present Invention

a. Illustrative Synthetic Schemes

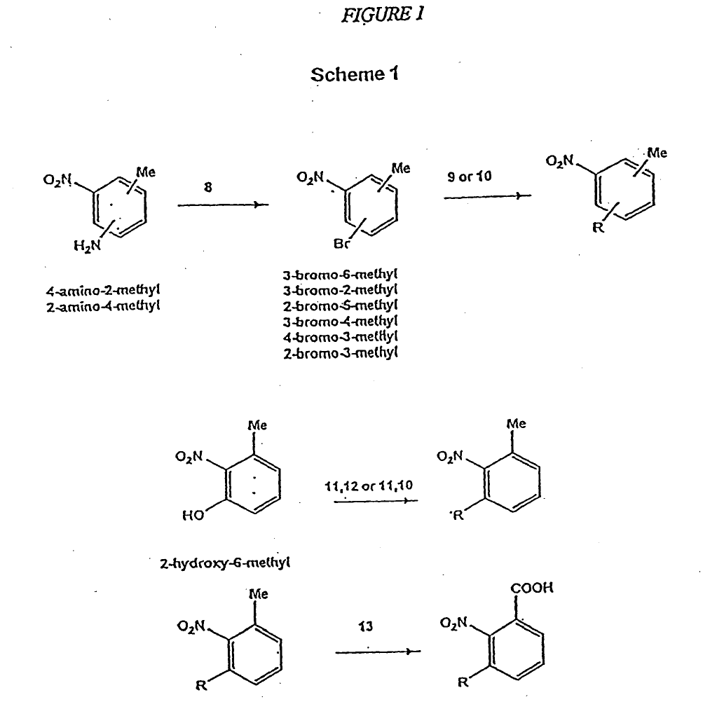

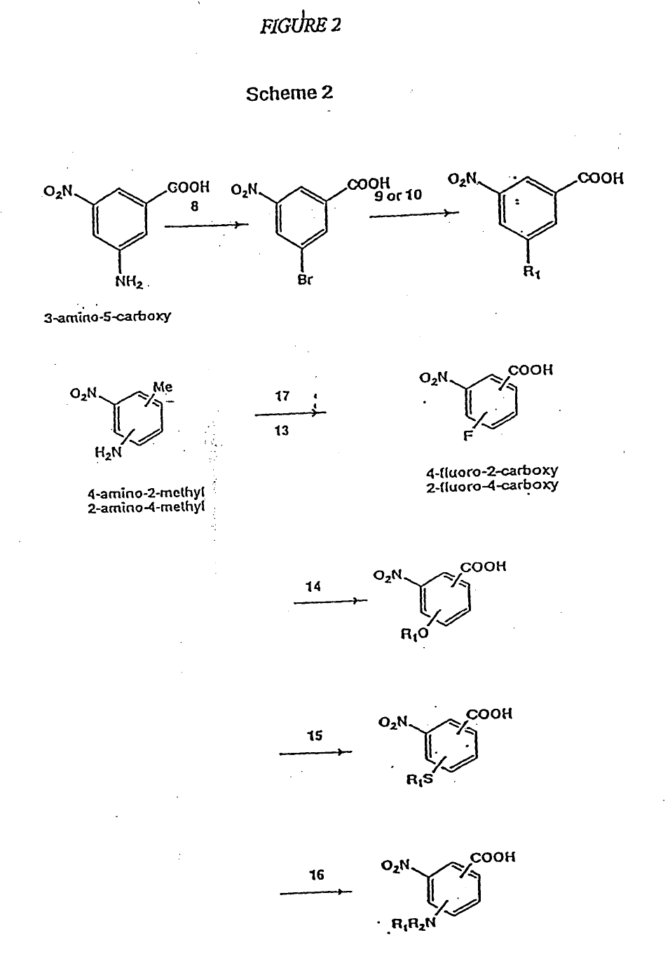

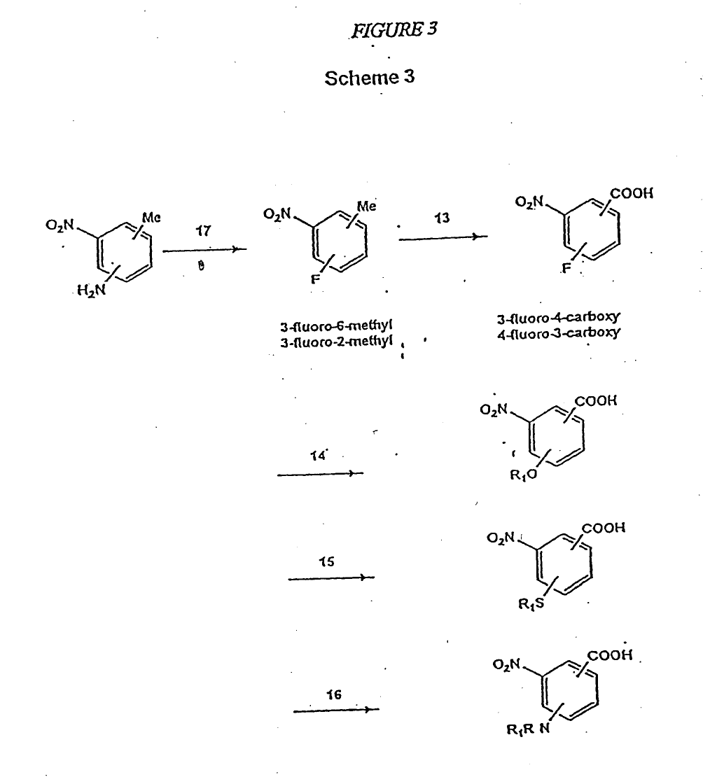

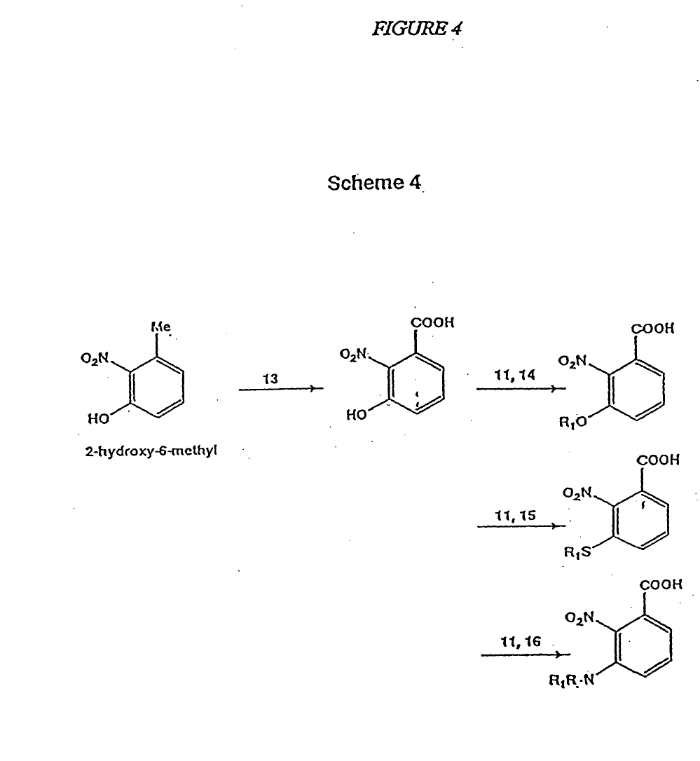

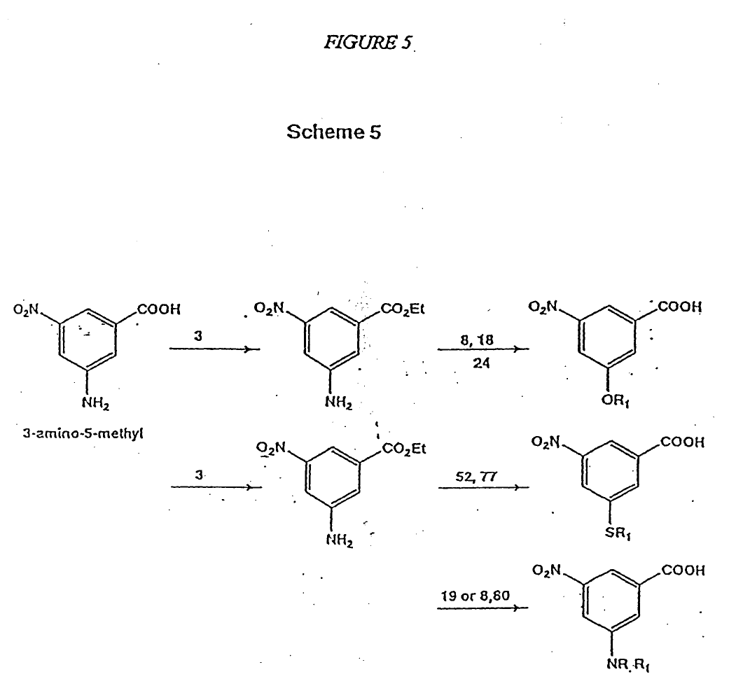

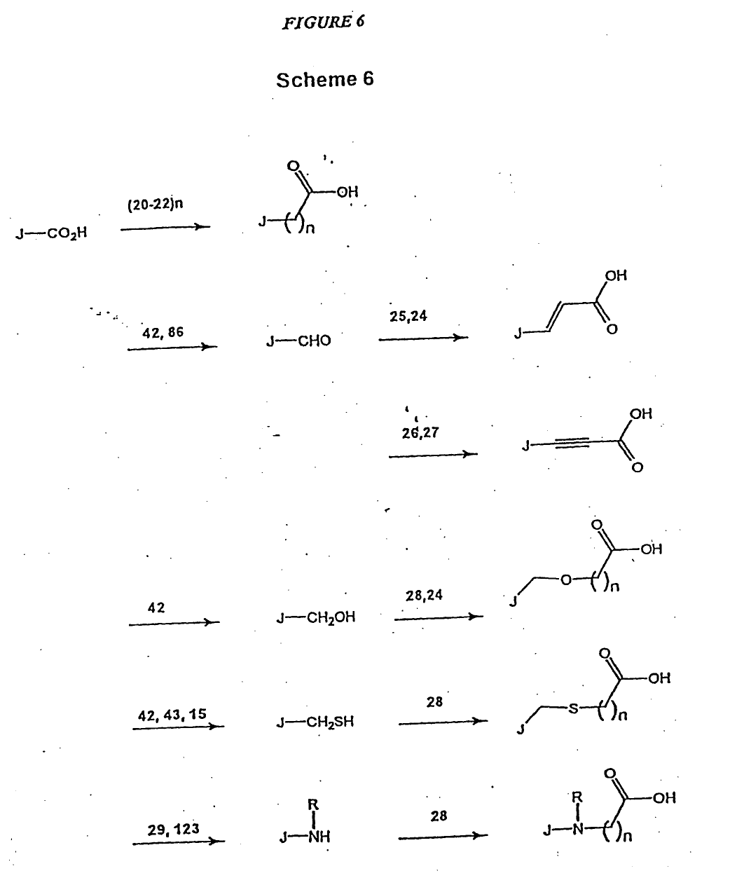

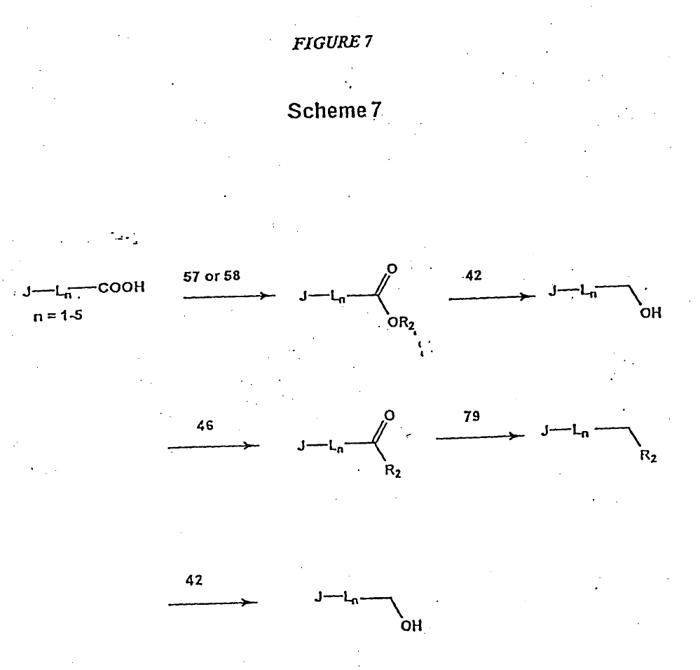

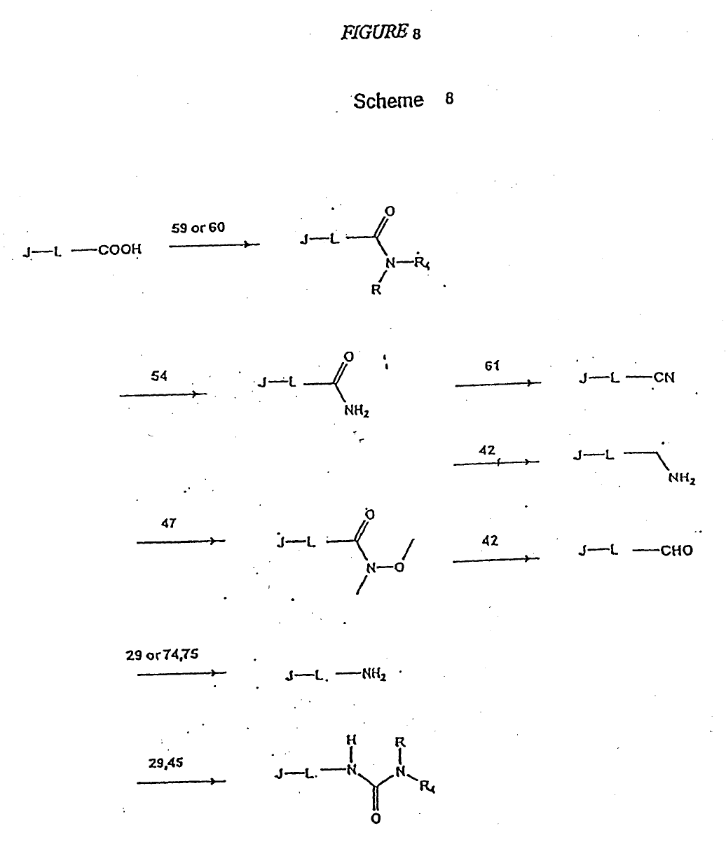

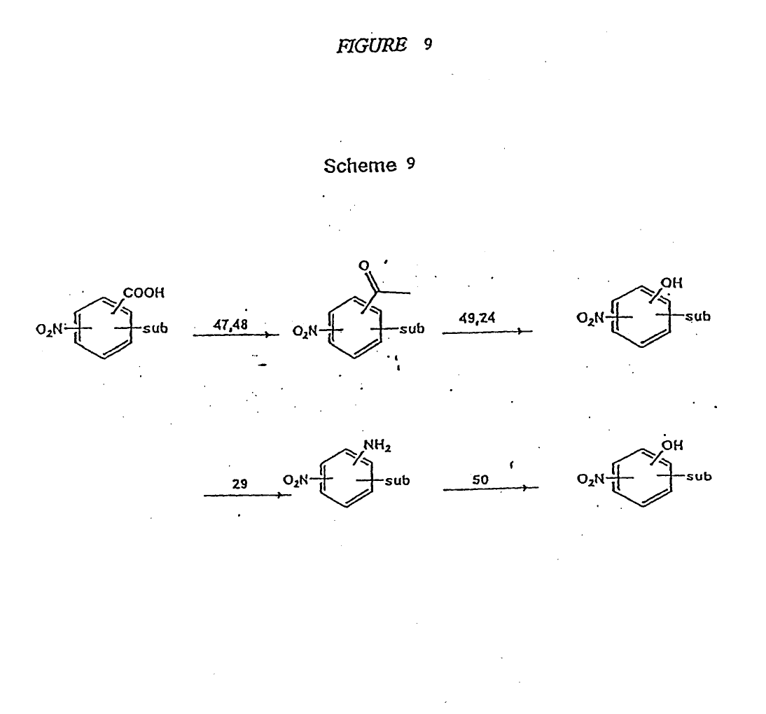

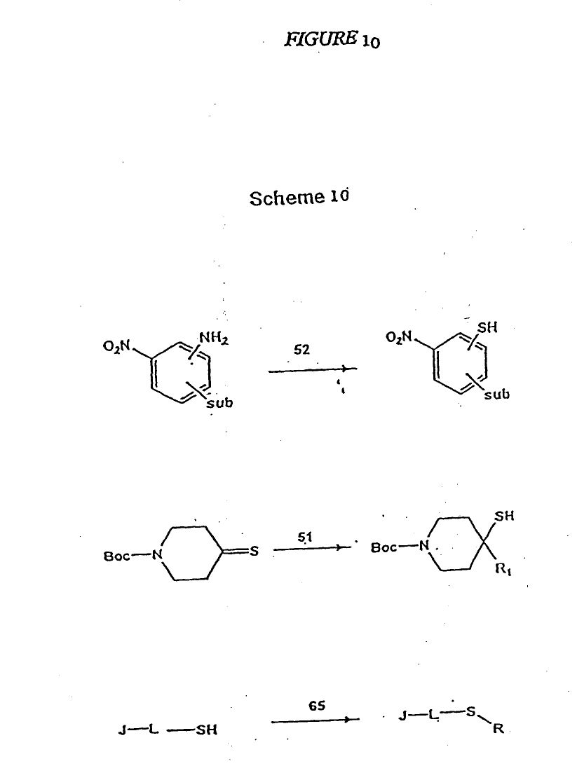

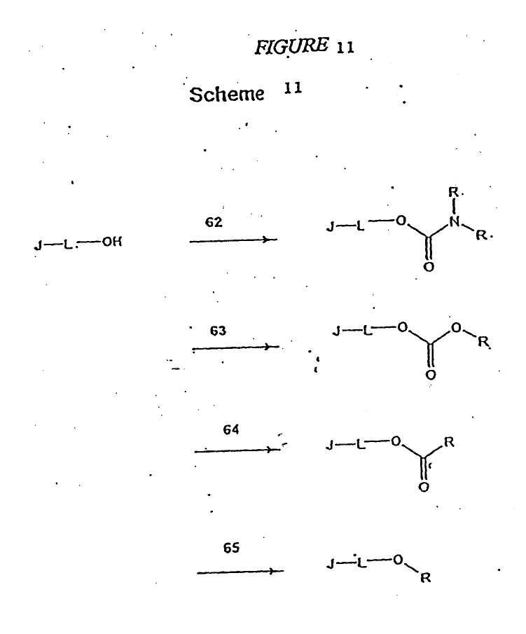

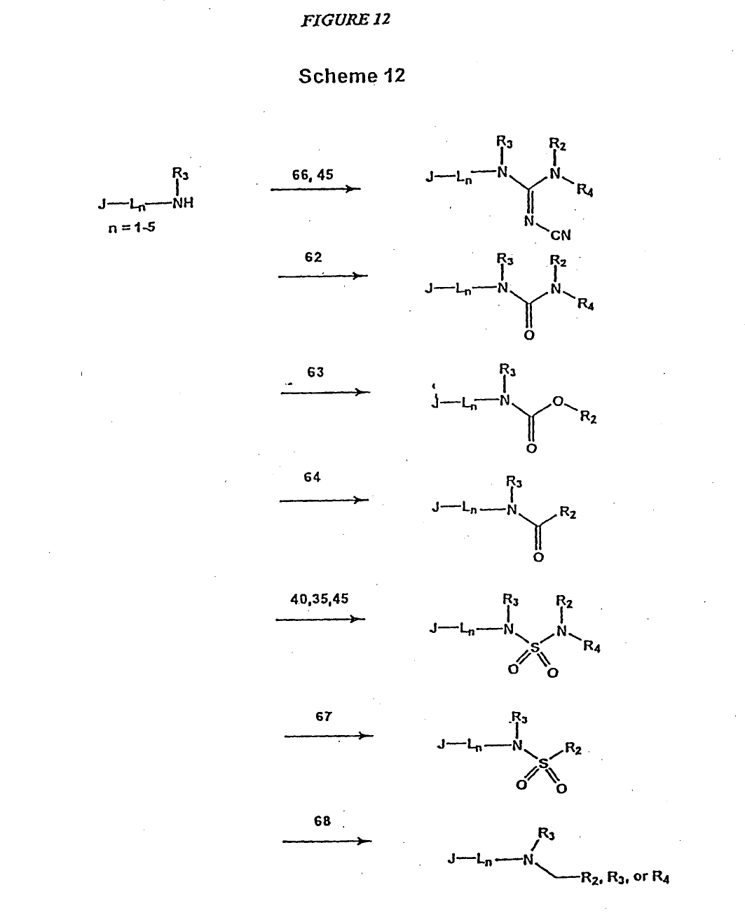

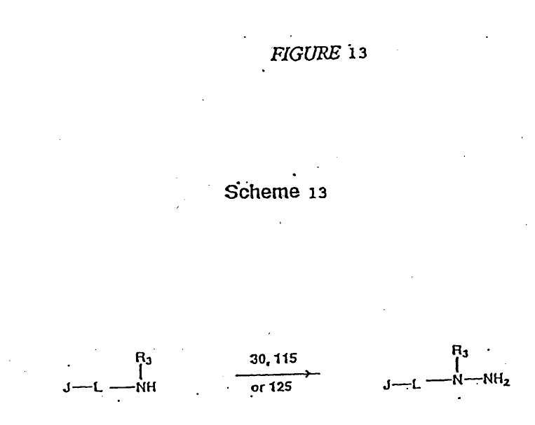

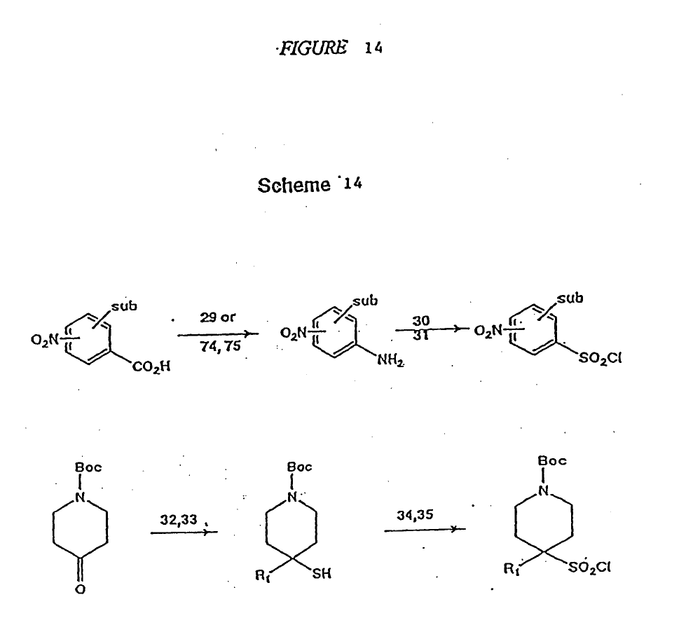

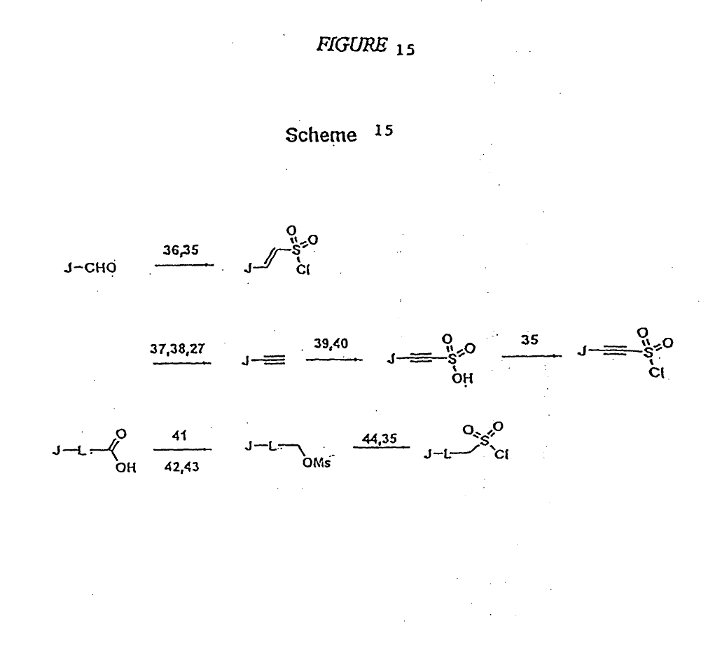

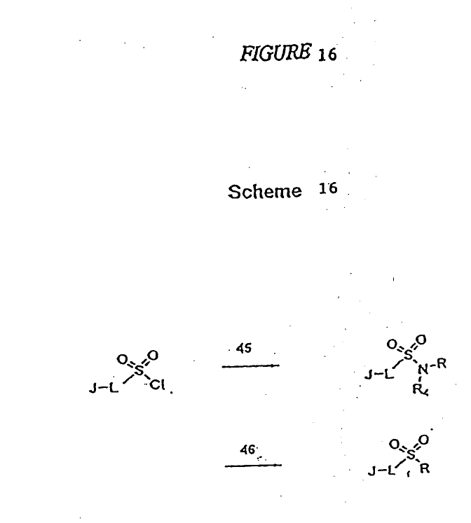

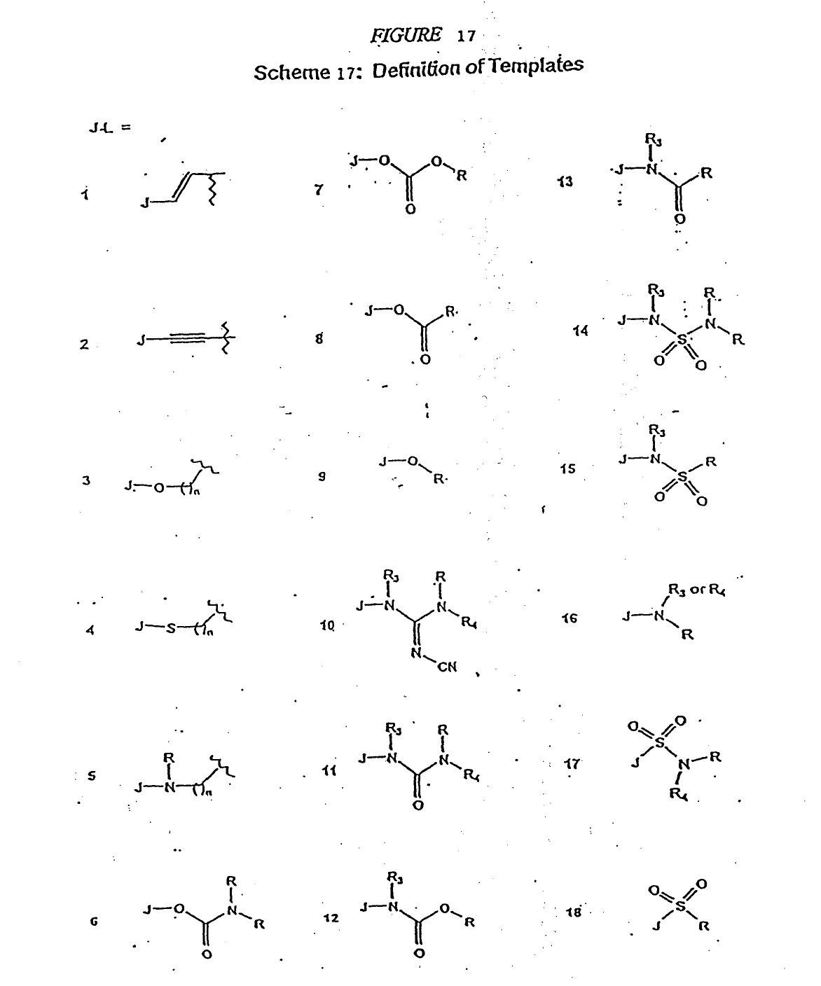

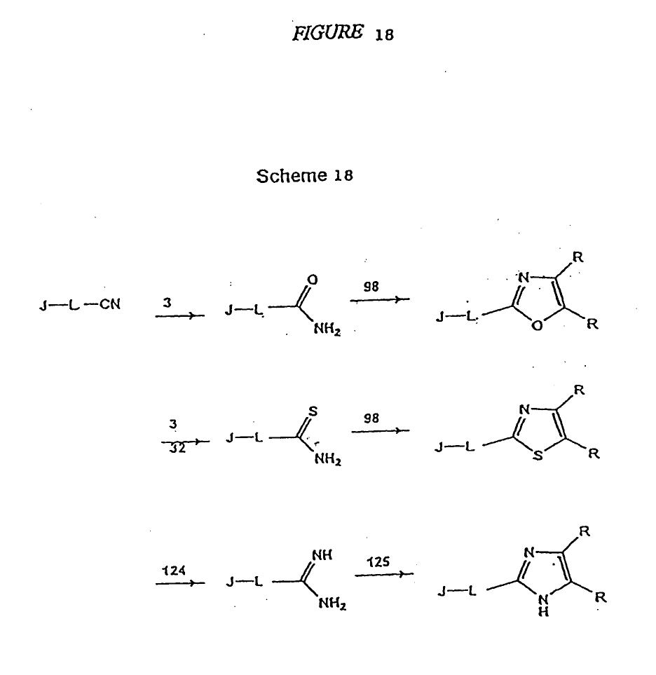

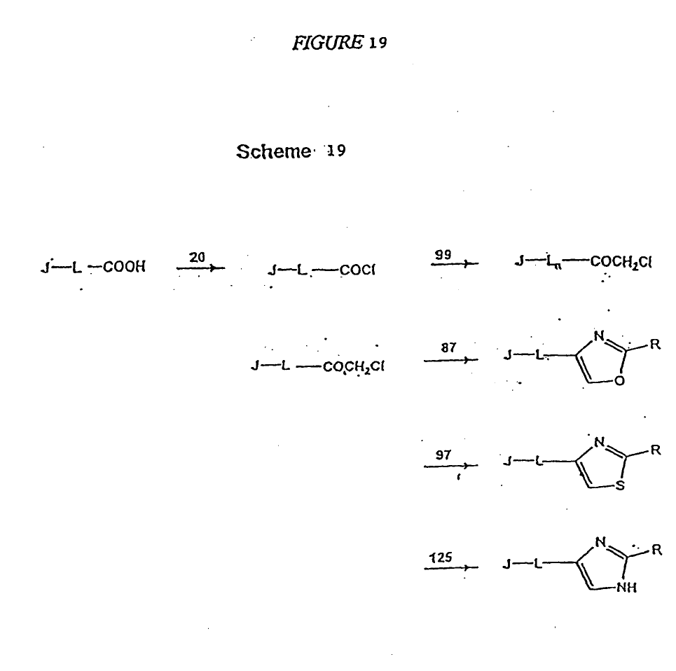

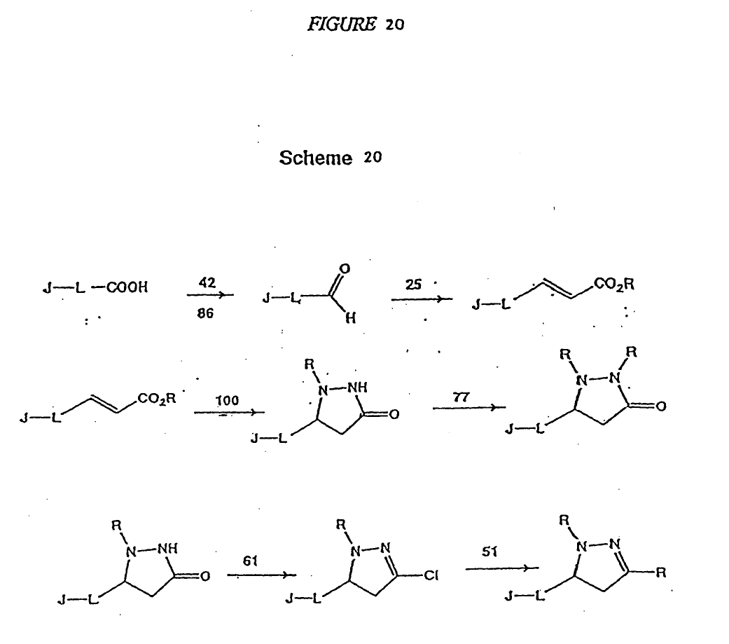

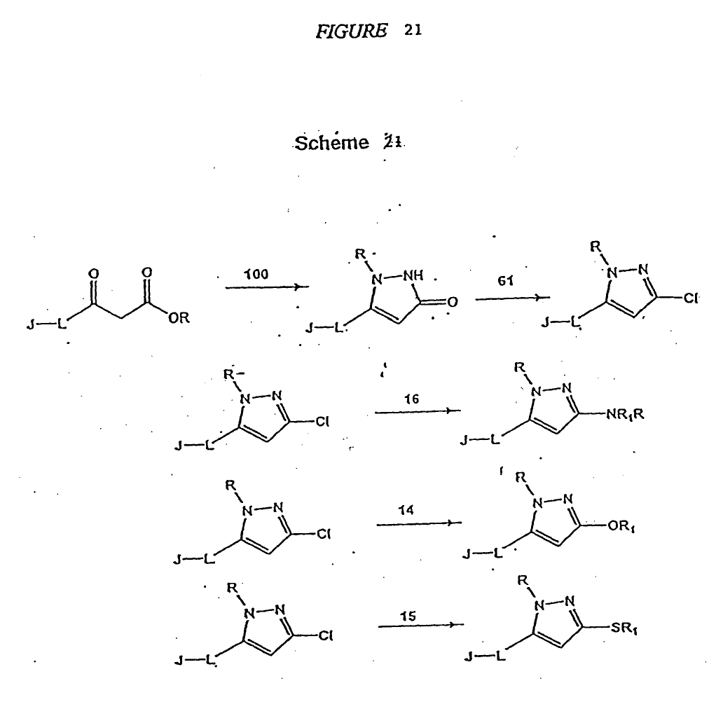

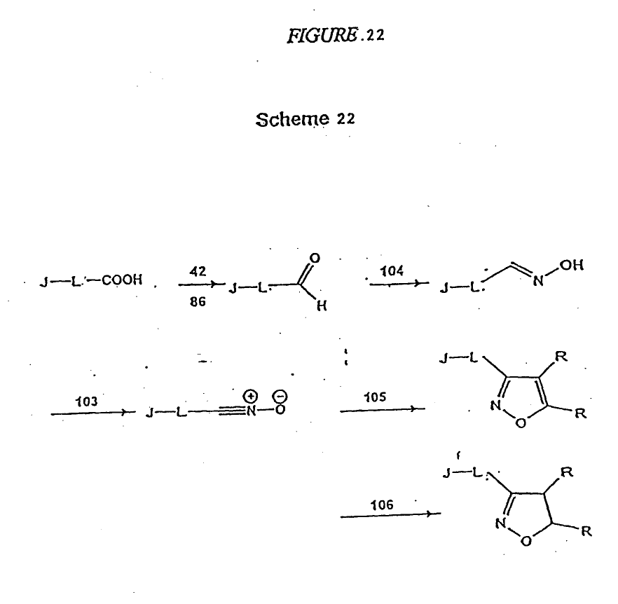

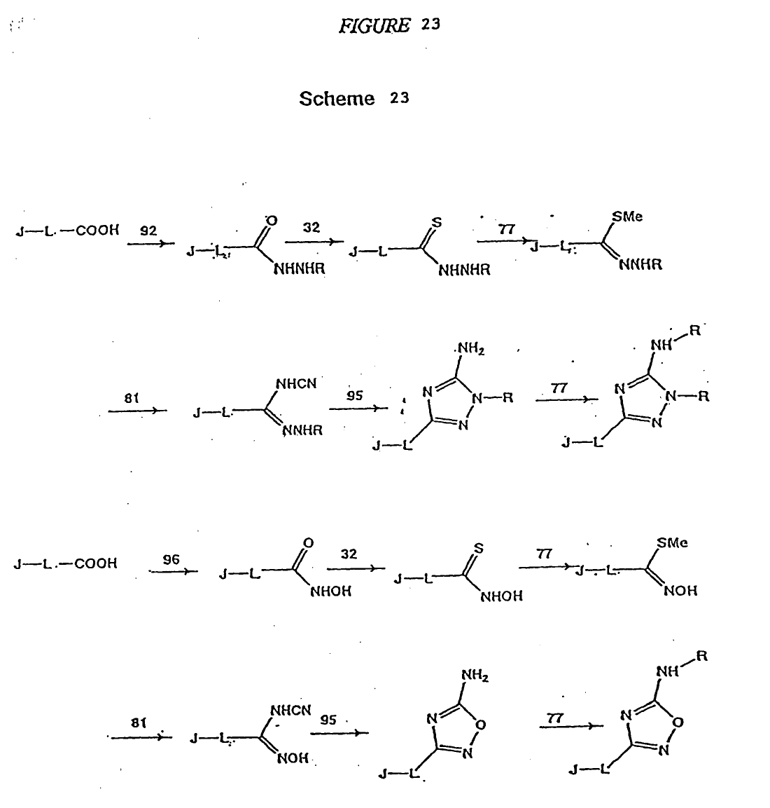

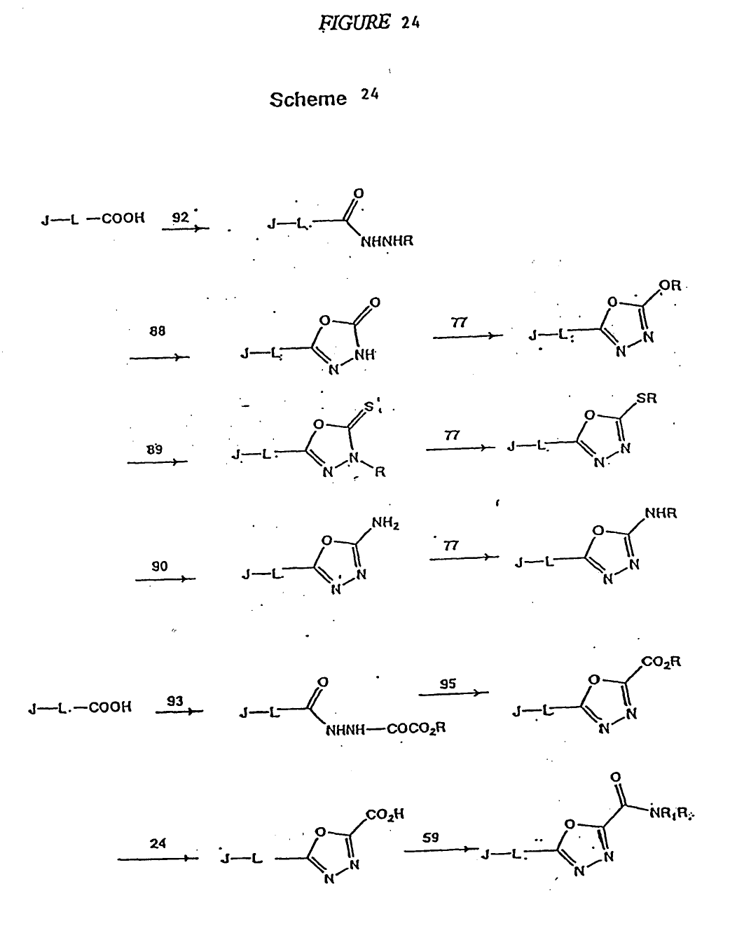

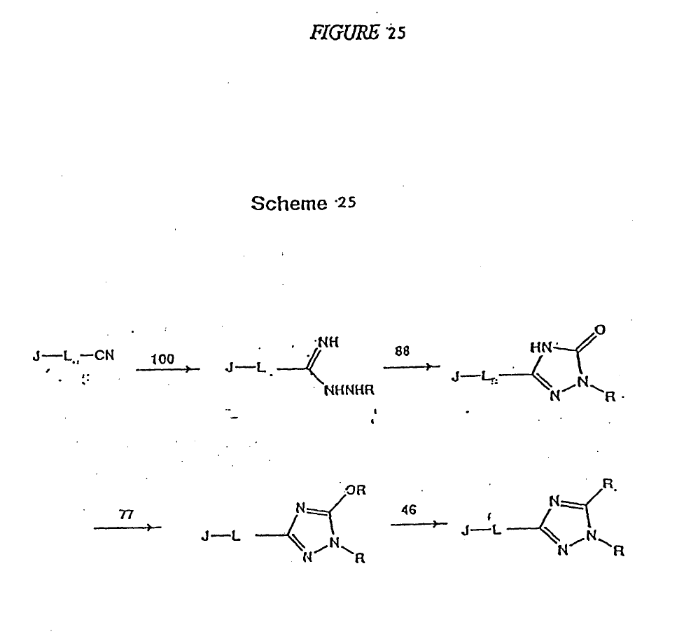

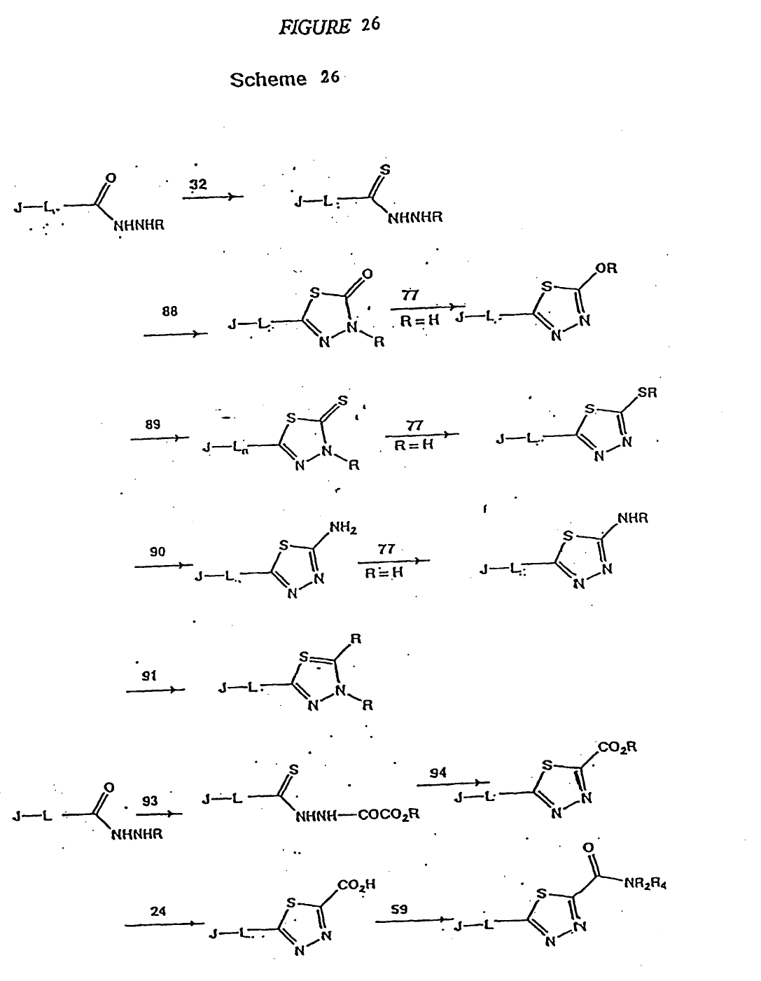

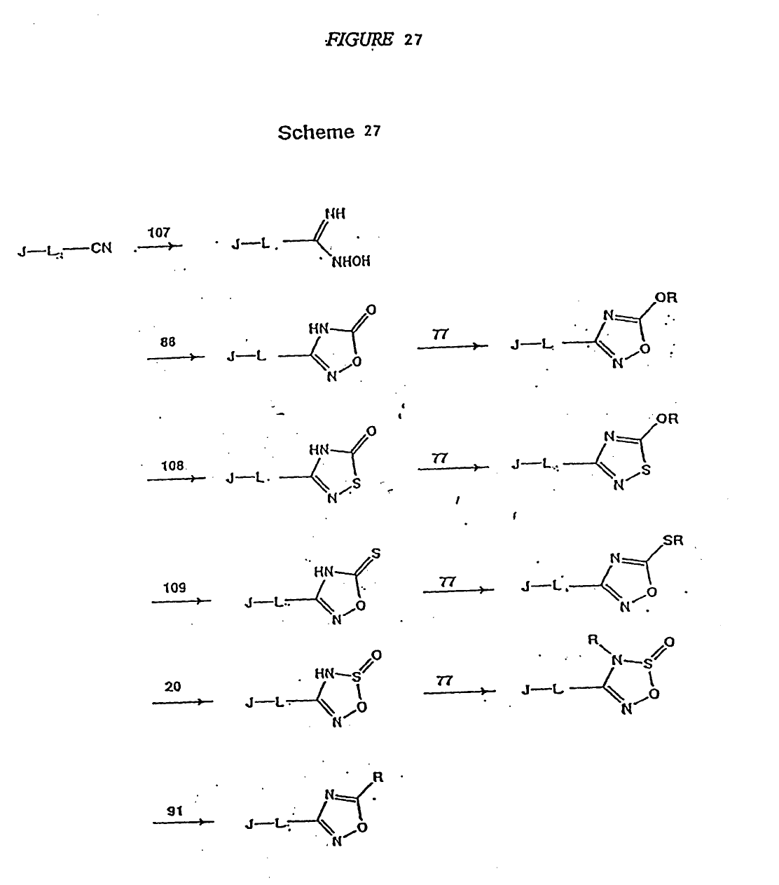

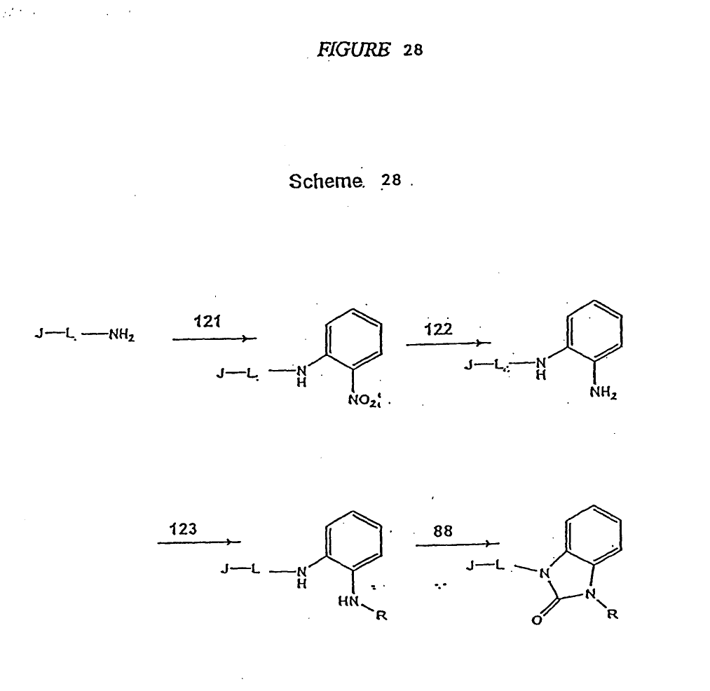

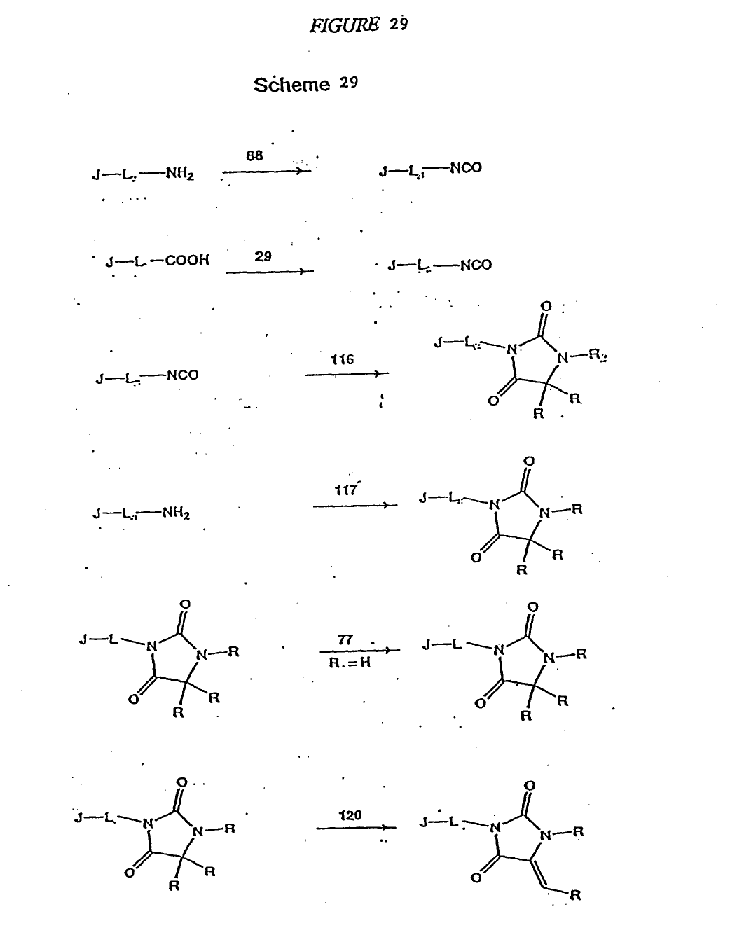

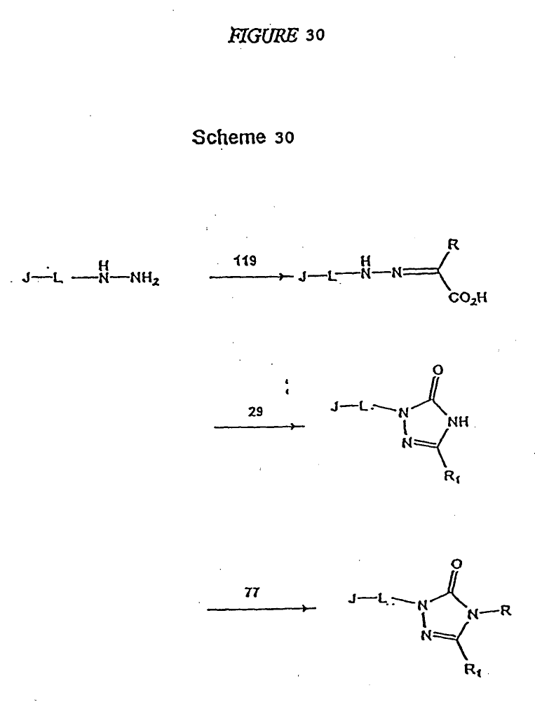

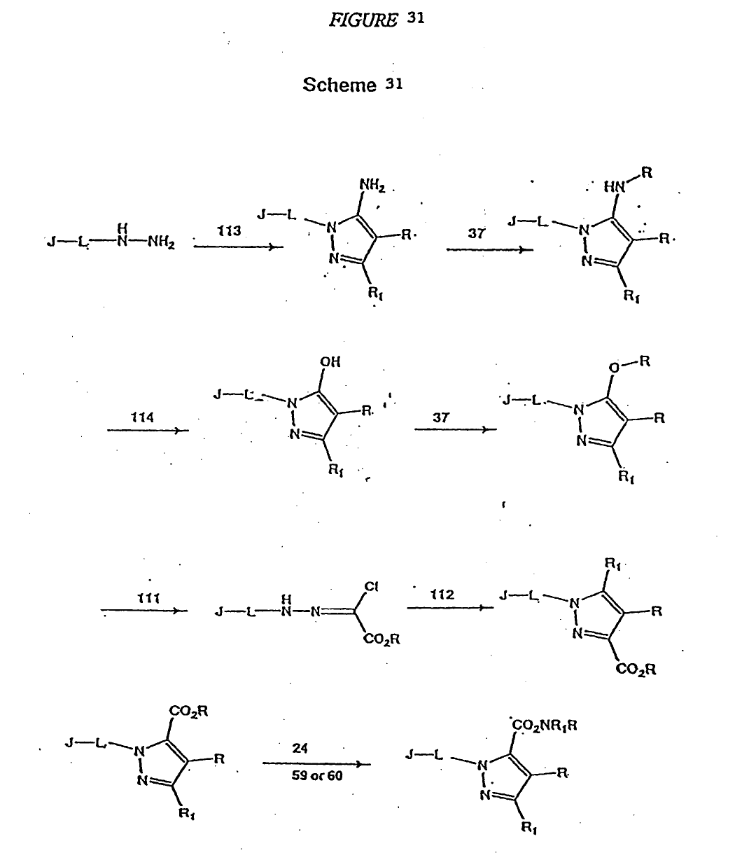

Exemplary synthesis schemes for generating prenyltransferase inhibitors useful in the methods and compositions of the present invention are shown in FIGS. 1-31.

The reaction conditions in the illustrated schemes of FIG. 1-31 are as follows:

1) R₁CH₂CN, NaNH₂, toluene

(Arzneim-Forsch, 1990, 40, 11, 1242)

2) H₂SO₄, H₂O, reflux

(Arzneim-Forsch, 1990,40, 11, 1242)

3) H₂SO₄, EtOH, reflux

(Arzneim-Forsch, 1990, 40, 11, 1242)

4) NaOH, EtOH, reflux

5) (Boc)₂O, 2M NaOH, THF

6) LiHDMS, R₁X, THF

(Merck Patent Applic # WO 96/06609)

7) Pd—C, H₂, MeOH

8) t-BuONO, CuBr, HBr, H₂O

(J. Org. Chem. 1977, 42, 2426)

9) ArB(OH)₂, Pd(PPh₃)₄, Dioxane

(J. Med. Chem. 1996, 39, 217-223)

10) R₁₂(H)C═CR₁₃R₁₄, Pd(OAc)₂, Et₃N, DMF

(Org. React. 1982, 27, 345)

11) Tf₂O, THF

(J. Am. Chem. Soc. 1987, 109, 5478-5486)

12) ArSnBu₃, Pd(PPh₃)₄, Dioxane

(J. Am. Chem. Soc. 1987, 109, 5478-5486)

13) KMnO₄, Py, H₂O

(J. Med. Chem. 1996, 39, 217-223)

14) NaOR₁, THF

15) NaSR₁, THF

16) HNR₁R₁₃, THF

17) HONO, NaBF₄

(Adv. Fluorine Chem. 1965,4, 1-30)

18) Pd(OAc)₂, NaH, DPPF, PhCH₃, R₁OH

(J. Org. Chem. 1997, 62, 5413-5418)

19) i. R₁X, Et₃N, CH₂Cl₂, ii. R₁₃X

20) SOCl₂, cat DMF

21) CH₂N₂, Et₂O

22) Ag₂O, Na₂CO₃, Na₂S₂O₃, H₂O

(Tetrahedron Lett. 1979, 2667)

23) AgO₂CPh, Et₃N, MeOH

(Org. Syn., 1970, 50, 77; J. Am. Chem. Soc. 1987, 109, 5432)

24) LiOH, THF-MeOH

25) (EtO)₂P(O)CH₂CO₂R, BuLi, THF

26) MeO₂CCH(Br)═P(Ph)₃, benzene

27) KOH or KOtBu

28) Base, X(CH₂)ₙCO₂R

29) DPPA, Et₃N, toluene

(Synthesis 1985, 220)

30) HONO, H₂O

31) SO₂, CuCl, HCl, H₂O

(Synthesis 1969, 1-10, 6)

32) Lawesson's reagent, toluene

(Tetrahedron Asym. 1996, 7, 12, 3553)

33) R₂M, solvent

34) 30% H₂O₂, glacial CH₃CO₂H

(Helv. Chim. Acta. 1968, 349, 323)

35) triphosgene, CH₂Cl₂

(Tetrahedron Lett., 1996, 37, 8589)

36) i. (EtO)₂P(O)CHLiSO₂Oi-Pr, THF, ii. NaI

37) Ph₃PCH₃I, NaCH₂S(O)CH₃, DMSO

(Synthesis 1987, 498)

38) Br₂, CHCl₃ or other solvent

(Synthesis 1987, 498)

39) BuLi, Bu₃SnCl

40) ClSO₂OTMS, CCl₄

(Chem. Ber. 1995, 128, 575-580)

41) MeOH—HCl, reflux

42) LAH, Et₂O or LiBH₄, EtOH or BH₃-THF

(Tetrahedron Lett., 1996, 37, 8589)

43) MsCl, Et₃N, CH₂Cl₂

(Tetrahedron Lett., 1996, 37, 8589)

44) Na₂SO₃, H₂O

(Tetrahedron Lett., 1996, 37, 8589)

45) R₂R₄NH, Et₃N, CH₂Cl₂

46) R₂M, solvent

47) CH₃NH(OCH₃), EDC, HOBt, DIEA, CH₂Cl₂ or DMF

(Tetrahedron Lett, 1981, 22, 3815)

48) MeLi, THF

49) mCPBA, CH₂Cl₂

50) HONO, Cu₂O, Cu(NO₃)₂, H₂O

(J. Org. Chem. 1977,42, 2053)

51) R₁M, solvent

52) HONO, NaS(S)COEt, H₂O

(Org. Synth. 1947, 27, 81)

53) HSR₂ or HSR₄, CH₂Cl₂

54) i-BuOC(O)Cl, Et₃N, NH₃, THF

55) R₂R₄NH, CH₂Cl₂, NaBH(OAc)₃

56) R₂R₄NH, MeOH/CH₃CO₂H, NaBH₃CN

57) R₂OH, EDC, HOBt, DIEA, CH₂Cl₂ or DMF

58) R₂OH, HBTU, HOBt, DIEA, CH₂Cl₂ or DMF

59) R₂R₄NH, EDC, HOBt, DIEA, CH₂Cl₂ or DMF

60) R₂R₄NH, HBTU, HOBt, DIEA, CH₂Cl₂ or DMF

61) POCl₃, Py, CH₂Cl₂

62) R₂R₄NCO, solvent

63) R₂OC(O)Cl, Et₃N, solvent

64) R₂CO₂H, EDC or HBTU, HOBt, DIEA, CH₂Cl₂ or DMF

65) R₂X, Et₃N, solvent

66) (CH₃S)₂C═N(CN), DMF, EtOH

(J. Med. Chem. 1994, 37, 57-66)

67) R₂SO₂Cl, Et₃N, CH₂Cl₂

68) R₂— or R₃— or R₄CHO, MeOHWCH₃CO₂H, NaBH₃CN

(Synthesis 1975, 135-146)

69) Boc(Tr)-D or L-CysOH, HBTU, HOBt, DIEA, CH₂Cl₂ or DMF

70) Boc(Tr)-D or L-CysH, NaBH₃CN, MeOH/CH₃CO₂H

(Synthesis 1975, 135-146)

71) S-Tr-N-Boc cysteinal, ClCH₂CH₂Cl or THF, NaBH(OAc)₃

(J. Org. Chem. 1996, 61, 3849-3862)

72) TFA, CH₂Cl₂, Et₃ SiH or (3:1:1) thioanisole/ethanedithiol/DMS

73) TFA, CH₂Cl₂

74) DPPA, Et₃N, toluene, HOCH₂CH₂SiCH₃

(Tetrahedron Lett. 1984, 25, 3515)

75) TBAF, THF

76) Base, TrSH or BnSH

77) Base, R₂X or R₄X

78) R₃NH₂, MeOH/CH₃CO₂H, NaBH₃CN

79) N₂H₄, KOH

80) Pd₂(dba)₃, P(o-tol)₃, RNH₂, NaOtBu, Dioxane, R₁NH₂

(Tetrahedron Lett. 1996, 37, 7181-7184).

81) Cyanamide.

82) Fmoc-Cl, sodium bicarbonate.

83) BnCOCl, sodium carbonate.

84) AllylOCOCl, pyridine.

85) Benzyl bromide, base.

86) Oxalyl chloride, DMSO.

87) RCONH₂.

88) Carbonyldiimidazole, neutral solvents (e.g., DCM, DMF, THF, toluene).

89) Thiocarbonyldiimidazole, neutral solvents (e.g., DCM, DMF, THF, toluene).

90) Cyanogen bromide, neutral solvents (e.g., DCM, DMF, THF, toluene).

91) RCOCl, Triethylamine

92) RNHNH₂, EDC.

93) RO₂CCOCl, Et₃N, DCM.

94) MSOH, Pyridine (J. Het. Chem., 1980, 607.)

95) Base, neutral solvents (e.g., DCM, toluene, THF).

96) H₂NOR, EDC.

97) RCSNH₂.

98) RCOCHBrR, neutral solvents (e.g., DCM, DMF, THF, toluene), (Org. Proc. Prep. Intl., 1992, 24, 127).

99) CH₂N₂, HCl. (Synthesis, 1993, 197).

100) NH₂NHR, neutral solvents (e.g., DCM, DMF, THF, toluene).

101) RSO₂Cl, DMAP. (Tetrahedron Lett., 1993, 34, 2749).

102) Et₃N, RX. (J. Org. Chem., 1990, 55, 6037).

103) NOCl or Cl₂ (J. Org. Chem., 1990, 55, 3916).

104) H₂NOH, neutral solvents (e.g., DCM, DMF, THF, toluene).

105) RCCR, neutral solvents (DCM, THF, Toluene).

106) RCHCHR, neutral solvents (DCM, THF, Toluene).

107) H₂NOH, HCl.

108) Thiocarbonyldiimidazole, SiO₂ or BF₃OEt₂. (J. Med. Chem., 1996, 39, 5228).

109) Thiocarbonyldiimidazole, DBU or DBN. (J. Med. Chem., 1996, 39, 5228).

110) HNO₂, HCl.

111) ClCH₂CO₂Et (Org. Reactions, 1959, 10,143).

112) Morpholine enamine (Eur. J. Med. Chem., 1982, 17, 27).

113) RCOCHR′CN

114) RCOCHR′CO₂Et

115) Na₂SO₃

116) H₂NCHRCO₂Et

117) EtO₂CCHRNCO

118) RCNHNH₂.

119) RCOCO₂H, (J. Med. Chem., 1995, 38, 3741).

120) RCHO, KOAc.

121) 2-Fluoronitrobenzene.

122) SnCl₂, EtOH, DMF.

123) RCHO, NaBH₃CN, HOAc.

124) NH₃, MeOH.

125) 2,4,6-Me3PhSO₂NH₂.

126) Et₂NH, CH₂Cl₂

127) MeOC(O)Cl, Et₃N, CH₂Cl₂

128) R₂NH₂, EDC, HOBT, Et₃N, CH₂Cl₂

129) DBU, PhCH₃

130) BocNHCH(CH₂STr)CH₂NH₂, EDC, HOBT, Et₃N, CH₂Cl₂

131) R₂NHCH₂CO₂Me, HBTU, HOBT, Et₃N, CH₂Cl₂

132) BocNHCH(CH₂STr)CH₂OMs, LiHMDS, THF

133) R₂NHCH₂CO₂Me, NaBH(OAc)₃, ClCH₂CH₂CI or THF

134) R₂NHCH₂CH(OEt)₂, HBTU, HOBT, Et₃N, CH₂Cl₂

135) NaBH(OAc)₃, ClCH₂CH₂Cl or THF, ACOH.

136) Piperidine, DMF.

137) Pd(Ph₃P)₄, Bu₃SnH.

138) RCO₂H, EDC, HOBT, Et₃N, DCM.

139) RNH₂, neutral solvents.

140) RCHO, NaBH₃CN, HOAc.

141) RNCO, solvent.

142) RCO₂H, EDC or HBTU, HOBt, DIEA, CH₂Cl₂ or DMF.

143) RCOCl, Triethylamine

144) RSO₂Cl, Et₃N, CH₂Cl₂.

145) SnCl₂, EtOH, DMF.

146) RNH₂, EDC, HOBt, DIEA, CH₂Cl₂ or DMF.

147) Dibromoethane, Et₃N, CH₂Cl₂

148) Oxalyl chloride, neutral solvents.

149) LiOH, THF-MeOH.

150) Carbonyldiimidazole, neutral solvents (e.g., DCM, DMF, THF, toluene).

151) RNH₂, Et₃N, CH₂Cl₂.

152) Base, RX.

153) DBU, PhCH₃

154) DPPA, Et₃N, toluene (Synthesis 1985, 220)

155) SOCl₂, cat DMF.

156) ArH, Lewis Acid (AlCl₃, SnCl₄, TiCl₄), CH₂Cl₂.

157) H₂NCHRCO₂Et, neutral solvents.

BocHNCHRCO₂H, EDC OR HBTU, HOBt, DIEA, CH₂Cl₂ or DMF.

159) TFA, CH₂Cl₂.

b. Illustrative Combinatorial Libraries

The compounds of the present invention, particularly libraries of variants having various representative classes of substituents, are amenable to combinatorial chemistry and other parallel synthesis schemes (see, for example, PCT WO 94/08051). The result is that large libraries of related compounds, e.g., a variegated library of compounds represented by formula I above, can be screened rapidly in high throughput assays in order to identify potential antifungal lead compounds, as well as to refine the specificity, toxicity, and/or cytotoxic-kinetic profile of a lead compound. For instance, simple turbidimetric assays (e.g., measuring the A₆₀₀ of a culture), or spotting compounds on fungal lawns, can be used to screen a library of the subject compounds for those having inhibitory activity toward a particular fungal strain.

Simply for illustration, a combinatorial library for the purposes of the present invention is a mixture of chemically related compounds which may be screened together for a desired property. The preparation of many related compounds in a single reaction greatly reduces and simplifies the number of screening processes which need to be carried out. Screening for the appropriate physical properties can be done by conventional methods.

Diversity in the library can be created at a variety of different levels. For instance, the substrate aryl groups used in the combinatorial reactions can be diverse in terms of the core aryl moiety, e.g., a variegation in terms of the ring structure, and/or can be varied with respect to the other substituents.

A variety of techniques are available in the art for generating combinatorial libraries of small organic molecules such as the subject antifungal. See, for is example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lerner et al. PCT publication WO93/20242). Accordingly, a variety of libraries on the order of about 100 to 1,000,000 or more diversomers of the subject antifungals can be synthesized and screened for particular activity or property.

In an exemplary embodiment, a library of candidate antifungal diversomers can be synthesized utilizing a scheme adapted to the techniques described in the Still et al. PCT publication WO 94/08051, e.g., being linked to a polymer bead by a hydrolyzable or photolyzable group e.g., located at one of the positions of the candidate antifungals or a substituent of a synthetic intermediate. According to the Still et al. technique, the library is synthesized on a set of beads, each bead including a set of tags identifying the particular diversomer on that bead. The bead library can then be “plated” on a lawn of fungi for which an inhibitor is sought. The diversomers can be released from the bead, e.g., by hydrolysis. Beads surrounded by areas of no, or diminished, fungal growth, e.g., a “halo”, can be selected, and their tags can be “read” to establish the identity of the particular diversomer.

A) Direct Characterization

A growing trend in the field of combinatorial chemistry is to exploit the sensitivity of techniques such as mass spectrometry (MS), for example, which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

B) Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

C) Divide-Couple-Recombine

In yet another embodiment, a variegated library of compounds can be provided on a set of beads utilizing the strategy of divide-couple-recombine (see, for example, Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

D) Combinatorial Libraries by Light-Directed, Spatially Addressable Parallel Chemical Synthesis

A scheme of combinatorial synthesis in which the identity of a compound is given by its locations on a synthesis substrate is termed a spatially addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

E) Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr et al. (1993) JACS 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-Sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

The structures of the compounds useful in the present invention lend themselves readily to efficient synthesis. For example, a starting cyclic amine may be linked to a solid support via its ring nitrogen atom, the substituents on the ring can be elaborated, for example, as described in greater detail below, the ring may be cleaved from the solid support, and the nitrogen may be coupled with an isocyanate, chloroformate, isothiocyanate, R₃XS(O)Cl, R₃XS(O)₂Cl, R₃XCH₂Br or another electrophilic reagent to complete the molecule. In this way, a wide variety of related compounds and derivatives as described above may be prepared rapidly and conveniently for testing, e.g., in a high-throughput assay.

c. Illustrative Identification of Other Compounds of the Present Invention

The schemes below depict representative synthetic pathways by which such compounds may be accessed, although many other pathways will be known to those of skill in the art. The following references describe reactions which may be useful in preparing compounds active as prenyltransferase inhibitors: Gaare, K. Repstad, T.; Bannache, T.; Undheim, K. Acta Chemica Scandanavica 1993, 47, 57-62; Yang, Y.; Wong, H. N. C. Tetrahedron 1994, 50, 9583-9608; J. Am. Chem. Soc. 1986, 108, 2662; J. Am. Chem. Soc. 1970, 92, 6644; J. Org. Chem. 1974, 39, 2778.

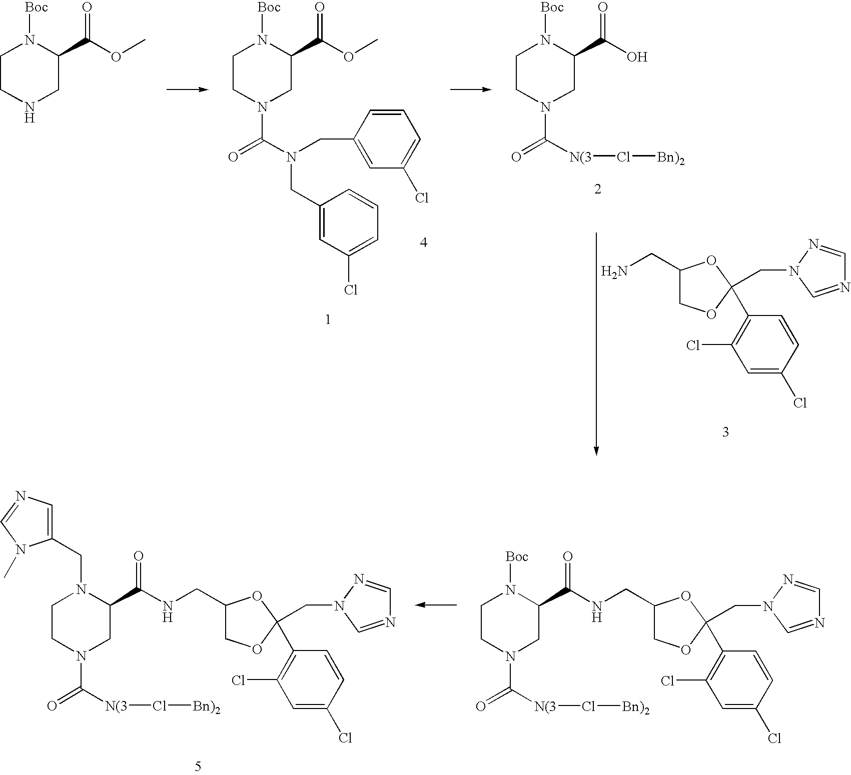

Compound A. To a solution of triphosgene (6.1 g, 21 mmol) in toluene (150 mL) at 0° C. was added a solution of 3-Cl dibenzylamine (12.5, 48 mmol) and pyridine (2 g, 25 mmol) in toluene (150 mL) dropwise. The reaction mixture was stirred at 0° C. for 2 h, poured into brine and extracted with CH₂Cl₂. The organic extracts were washed with brine, dried (MgSO₄) and concentrated to give the carbamoyl chloride (15.3 g).

To a solution of Boc-piperazine (1 g, 4.1 mmol) in CH₂Cl₂ (15 mL) was added pyridine (0.4 g, 4.9 mmol) followed by a solution of carbamoyl chloride in CH₂Cl₂ (5 mL). The reaction mixture was stirred at room temp for 16 h, poured into a solution of sat. NaHCO₃ and extracted with CH₂Cl₂. The organic extracts were dried and concentrated. The residue was purified by silica gel chromatography to give A (1.36 g).

Compound B. To a solution of 1 (1.3 g, 2.4 mmol) in 1:1 THF/MeOH (50 mL) was added a solution of 1 M LiOH (10 mL, 10 mmol). The reaction mixture was stirred is at room temp for 18 h, pored into excess 1M HCl solution and extracted with CH₂Cl₂. The organic extracts were dried and concentrated to give acid B (1.1 g).

Compound D. To a solution of acid B (1.1 g, 2 mmol) in CH₂Cl₂ (20 mL) was added amine C (0.69 g, 2.1 mmol) followed by DIPEA (0.47 mL, 2.5 mmol), EDC (0.48 g, 2.5 mmol) and HOBt (0.38 g, 2.5 mmol). The reaction mixture was stirred at room temp for 16 h, poured into a solution of sat. NaHCO₃ and extracted with CH₂Cl₂. The organic extracts were dried and concentrated. The residue was purified by silica gel chromatography to give D (1.38 g).

Compound E. To a solution of Boc-piperazine D in CH₂Cl₂ (10 mL) was added TFA (10 mL). The reaction mixture was stirred at room temp for 2 h. The solvents were removed under reduced pressure. The residue was dissolved in CH₂Cl₂ and washed with 2M NaOH soln. The organic layer was dried and concentrated to give 1.16 g of the deprotected piperazine. To a solution of the deprotected piperazine (1.16 g, 1.58 mmol) in dichloroethane (10 mL) was added acetic acid (0.5 mL) followed by 5-methylimidazole carboxaldehyde (0.27 g, 2.4 mmol). To this solution was added NaBH(OAc)₃ and the reaction mixture was stirred at room temp for 40 h. The reaction mixture was poured into 2M NaOH solution and extratcted with CH₂Cl₂. The organic extracts were dried and concentrated. The residue was dissolved in conc. HCl soln., basified with 2M NaOH soln. and extracted with CH₂Cl₂. The organic extracts were dried and concentrated. The residue was purified by preparative HPLC to give E (0.97 g).

Inhibition of Prenyltransferases

a. SAR of Prenyltransferase Inhibitors

As described below, a variety of different compounds were tested for inhibitory activity against human and Candida GGPTase. Table 1 provides Structure-Activity relationship (SAR) data for these different compounds. In addition to the assays for GGPTase described below, other assays for prenyltransferases may be found in: Reiss, Y., Goldstein, J. L., Seabra, M. C., Casey, P. J., and Brown, M. S. 1990. Cell 62:81-88; Zhang, F. L., Fu, H.-W., Casey P. J. and Bishop, W. R. 1996 Biochemistry 35:8166-8171; and Bishop, W. R. et al. 1995 J. Biol. Chem. 270:30611-30618.

b. Demonstration of the Effect of GGPTase Inhibitors on the Prenylation State of Newly Synthesized CARHO1.

(i) Methodology.

To look at the effect of GGPTase I inhibitors in vivo, a recombinant C. albicans strain engineered to express a Myc tagged CaRHO1 under the control of the C. albicans PCK1 promoter is used. This promoter is repressed by glucose and derepressed by gluconeogenic carbon sources such as succinate. It should also be possible to be look at the endogenous substrates of the GGPTase I. Cells are treated with a sublethal dose of compound for a period of time which has been established from a kill curve analysis in the appropriate media. After the treatment time, cells are harvested and whole cell extracts (WCE) made, these extracts are then resolved by high speed centrifugation into cytosolic and membrane fractions. Visualisation of the localisation of the MycCaRHO1 is achieved by SDS-PAGE and Western blotting. MycCaRHO1 that has been geranylgeranylated will be localised to the membrane whereas ungeranylgeranylated protein should be found in the cytosolic fraction. Treatment of cells with DMSO (mock) and GGPTase I inhibitor MycCaRHO1 will be apparent in the WCE and pellet fractions. In mock treated cells, MycCaRHO1 should be absent from the cytosolic fraction, whereas in GGPTase I inhibitor treated cells, some MycCaRHO1 should be apparent in the cytosolic fraction indicating that a proportion of the newly synthesized MycCaRHO1 has not been geranylgeranylated. FIG. 32 shows that this prediction is borne out.

(ii) Generation of the CaRHO1 Replacement Construct.

The 5′ and 3′ non-coding regions of CaRHO1 were generated by PCR and cloned into pBluescript KS— in which the CaRHO1 ORF was exactly replaced with a BamHI site. Into this vector (pSCaRHO1.5c23) a PCK1.CaURA3 cassette was inserted from pSCaPCK1.3c1 to generate pSCaRHO1.19c1. This vector was mutagenised to destroy one of the two BamHI sites (pSCaRHO1.22c22) into which the Myc tagged CaRHO1 ORF (from pSCaRHO1.20c58) was inserted. The sequence of the oligos used to generate the Myc tagged CaRHO1 ORF are:

| | |
|---|---|
| CaRHO1.13: | 5′ CCCGGGATCCTTACAAGACAACACATTTCTT 3′ |
| CaRHO1.13: | 5′ CCGGGATCCTTACATAATGTCTGAACAAAAATTGAT |
| | ATCAGAAGAAGATTTGGTTAACGG 3′ |

the sequence of the Myc tag is underlined and corresponds to the amino acid sequence EQKLISEEDL. This epitope is recognized by the commercially available 9E10 monoclonal antibody. The final vector designated pSCaRHO1.23c21, harbours of the 5′ non-coding region of CaRHO1, the CaURA3 selectable marker, the C. albicans PCK1 promoter directing the expression of the Myc-tagged CaRHO1 and the 3′ untranslated region of CaRHO1. The presence of the CaRHO1 5′ and 3′ regions should direct this cassette to one of the 2 WT alleles of CaRHO 1 by homologous recombination.

(iii) Generation of the C. albicans PCK1-MycCaRHO1 Strain

The PCK1-MycCaRHO1 replacement construct was excised by a BssHII digest from the parent plasmid pSCaRHO1.23c21. The desired fragment was gel purified prior to being transformed into the C. albicans strain CAF3-1. The method used for CAF3-1 transformation is a lithium acetate protocol (from U. of Minnesota C. ablicans web site: http://alces.med.umn.edu/candida/liac.html). The transformation mixture is then plated onto selective (−Ura glucose) plates and incubated at 30° C. for 3 days. Individual transformants that appear are restreaked for singles and then preserved as a glycerol stock. To ensure that the correct integrative event has occurred, southern analysis was carried out on several colonies. Those colonies that exhibited the correct genotype were retained.

The strain used for the work described here is referred to as DWY-BL2-058.

(iv) Growth and Treatment of Cells

Cells of strain DIY-BL2-058 were grown overnight in YNB supplemented with 1 μg/ml histidine, 2 μg/ml methionine, 2 μg/ml tryptophan, 200 μg/ml glutamine and 2% glucose at 220 rpm at 31° C. The cell number was then determined, cells were pelleted by centrifugation and resuspended in fresh media at a density of 1×10⁷ cells/ml and incubated as above. Cells were either treated with 14 μl DMSO alone or 14 μl of a 25.6 mg/ml stock of inhibitor in DMSO (3 μg/ml final concentration). After 3 hrs incubation cells were pelleted, washed twice and resuspended to the original volume with the following media: YNB supplemented with 1 μg/ml histidine, 2 μg/ml methionine, 2 μg/ml tryptophan, 200 μg/ml glutamine, 2% succinate and 0.05% glucose. The PCK1 promoter is repressed in the media containing 2% glucose. The switch in media to 2% succinate, 0.05% glucose partially derepresses the PCK1 promoter such that the MycCaRHO1 protein is not overproduced. DMSO or inhibitor is then again added to this new media and the cells incubated for a fuirther 5hrs. After the required incubation the cells are pelleted and frozen at −80° C.

(v) Generation and Fractionation of Cellular Extracts

To generate cellular extracts, 10× TE supplemented with a protease inhibitors cocktail was added at 3-4 volumes of the pellet size (about 200 μl) and glass beads (425-600 microns; Sigma) were added to the meniscus. This mixture was then subjected to 5 1′ pulses in a bead beater with 2′ on ice between pulses. The mixture was then centrifuged at 3000 rpm to pellet cellular debris and the supernatent removed. The beads were washed with an equal volume of buffer and the supernatent added to the initial sample. This whole cell extract (WCE) was again centrifuged at 3000 rpm and the supernatent removed into a fresh tube. 50 μl of this WCE was subjected to high speed centrifugation (54000 rpm for 1 hr in a TI120.1 rotor) to resolve the membrane and cytosolic fractions. The cytosolic fraction was carefully removed. The membrane pellet fraction was washed with buffer and resuspended in 1× loading buffer. All fractions were frozen at −80° C.

(vi) SDS-PAGE and Western Blotting

Fractions were thawed on ice. The protein concentration was determined using the standard Bradford method for the WCEs and cytosolic fraction. 30 μg of protein were loaded for both the WCE and cytosolic fractions. For the membrane fraction, a volume equal to that loaded for the cytosolic fraction was loaded. Prior to loading, all fractions were boiled for 3′ with loading dye. Standard procedures were employed for the SDS.PAGE and Western blotting.

To analyse the Western blot, the blot was pre-blocked with 4% fat free milk in PBST. The 9E10 monoclonal anti-myc epitope antibody (available from Calbiochem) was incubated with the blot overnight at 4° C. at a concentration recommended by the manufacturers. The primary antibody was removed and the blot was washed 3×15′ with PBST. The blot is then incubated with 2° antibody which was goat anti-mouse HRP conjugated antibody for 1 hr at room temperature. The 2° antibody is removed and the blot washed again with 3×15′ with PBST and developed using the Pierce luminescent kit according to the manufacturers instructions.

As shown in FIG. 32, exposure of cells to a GGPTase I inhibitor increases the abundance of MycCaRHO1 in the cytosolic fraction (inhibitor-treated cells) but not of mock (DMSO) treated cells. Numbers 1-6 indicate the lanes of the gel which are denoted as W, whole cell extract, C, cytosolic fraction and P, pellet fraction. Protein molecular weight markers are indicated.

c. In vitro Assays of Fungal GGPTase Inhibitors

(i) Assay Protocol for Determining IC₅₀

Plate test compounds (10 μL per well) at predetermined concentration in 50% DMSO. For background control (blank) and reaction control (negative), add 10 μL of 200 μM GGPP and 10 μL 50% DMSO, respectively. Prepare assay buffer: 50 mM Tris, pH7.5, 20 mM KCL, 5 mM MgCl₂, 5 μM ZnCl₂, 0.5 mM Zw(3-14), 2 mM DTT and 0.1 mg/mL BSA.

Add 20 μL of C. albicans GGPTase and ³H-GGPP in assay buffer to test compound. Preincubate enzyme and ³H-GGPP with test compound for 15 minutes at room temperature. Add 20 μL C. albicans Rho in assay buffer. Incubate for 30 minutes at room temperature. Final assay conditions are 2 nM C. albicans GGPTase, 250 nM ³H-GGPP and 250 nM C. albicans Rho.

Add 100 μL 15 mM GGPP, 50 mM Tris, pH 7.0 and 2% BSA to quench reaction. Transfer reaction to Nickel chelate FlashPlate. Allow his-tagged C. albcians Rho to capture onto plate. Rinse plate 1× with 200 μL 20 mM Tris, pH 7.0. Read in TOPCOUNT.

(ii) In vitro Susceptibility Testing of Compounds in C. albicans

1: Innoculate strain C. albicans strain such as SC5314 into 20 mL of the appropriate medium and incubate at 35° C. with shaking (220 rpm) overnight

2: Count the C. albicans cells in a 1:10 dilution of the overnight culture using a haemocytometer.

3: Work out the dilution factor required to bring the cell number to 1×10³ cells/100 μL (equivalent to 1×10⁴ cells/mL) then add the required volume of the overnight culture to 25 mL media in a falcon tube.

4: Vortex the diluted cells and immediately pipette 100 μL of the cell suspension to each of the required rows of a 96 well plate using the multipipettor

5: Prepare each of the 100× stock solutions for the compounds to be tested in DMSO in the required concentration range in Eppendorf tubes.

6: The dilution series for each of the compounds may now be prepared in sequence:

For each compound—start with highest dilution. Add 10 μL compound in DMSO to the 490 μL of appropriate media. Immediately vortex and add 100 μL to the appropriate row of cells on the 96-well plate. Repeat this process for the next and subsequent concentrations of this compound before starting on the dilution series for additional compounds.

7: When complete cover the 96-well plate with an acetate sheet and incubate at 35° C. Inspect visually and record results for both plates at 24 hr and 48 hr. The MIC corresponds to the concentration of compound where no visible growth is observed.

(iii) Determination of Minimum Fungicidal Concentrations (MFC)

After the required time course for the MIC determination, the minimum fungicidal concentration can then be determined by plating out the entire contents of the well of the microtitre plates onto YPD or Sabourand plates. These plates are then incubated at 35° C. for 24-48 hrs. The MFC corresponds to the concentration of compound where no cellular growth is observed on the plate.

(iv) Assay Protocol for Determining Cytotoxicity of GGPTase Inhibitors in Human Cells

(A) Plate out cells at predetermined concentration in a volume of 150 μl.

(B) Allow cells to adhere to plate for twenty-four hours

(C) Add compounds to cells at predetermined concentration (62.5 μg/mL down four-fold, 8 dilutions) n=2

(D) Cells are exposed to drug for 7 days for the IMR90 Cell Line, and a period of 3 days for the H460 Cell Line.

(E) 1.H460 Cells are fixed in TCA, rinsed, stained with Sulforhodamine B stain, and the stain is solubilized for a final OD read.

2. IMR90 Cells have 3-{4,5-Dimethylthiazol-2-yl}-2,5-diphenyltetrazolium bromide (MTT) added to them for three hours prior to final read out. After the three hours, media and MTT are removed and MTT crystals are solubilized in 100% DMSO for final OD read.

A number of compounds were tested as described above, and the results are presented in Table 1:

TABLE 1

| | | CaGGTase | C. alb MIC | C. alb MFC |
|---|---|---|---|---|
| # | STRUCTURE | μM | μg/mL | μg/mL |
| 1 | 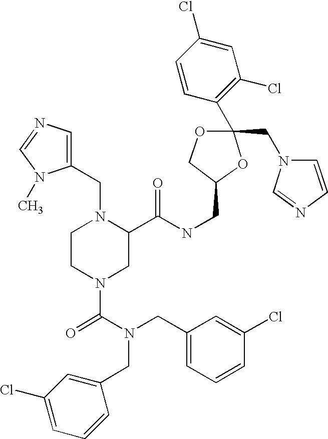 | <0.1 | <10 | <10 |
| 2 | 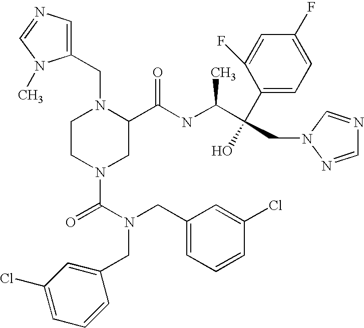 | <0.1 | <10 | <10 |
| 3 | 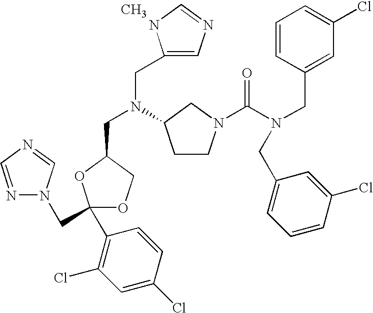 | <0.1 | <10 | <10 |
| 4 | 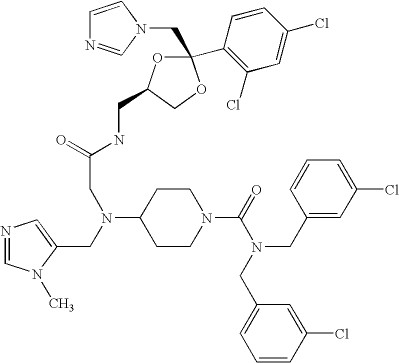 | <0.1 | <10 | <10 |
| 5 | 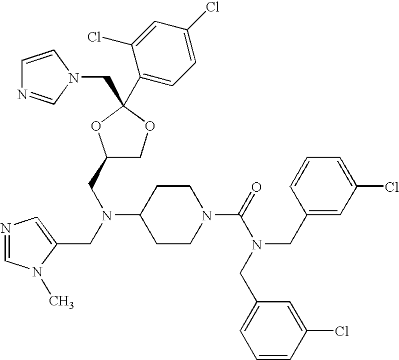 | <0.1 | <10 | <10 |
| 6 | 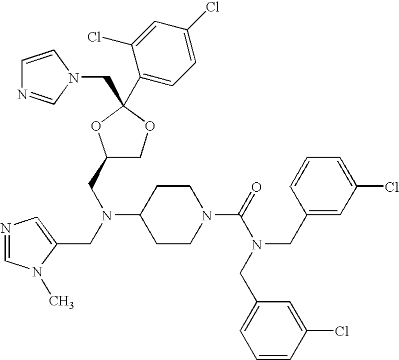 | <0.1 | <10 | <10 |
| 7 | 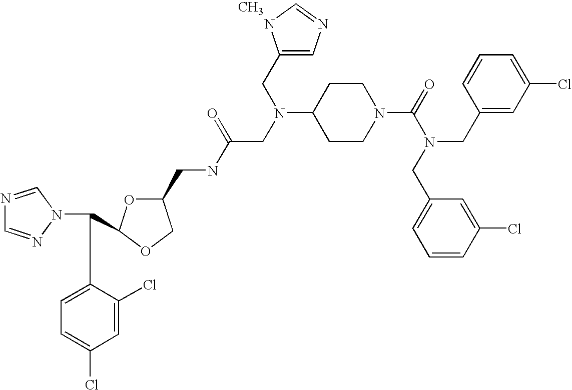 | <0.1 | <10 | <10 |
| 8 | 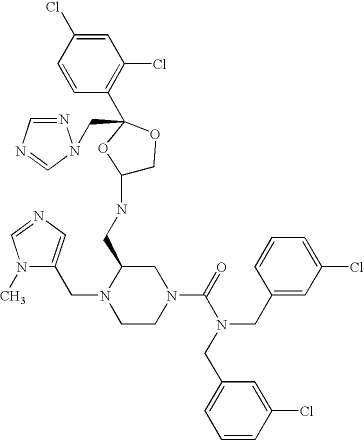 | <0.1 | <10 | <10 |
| 9 | 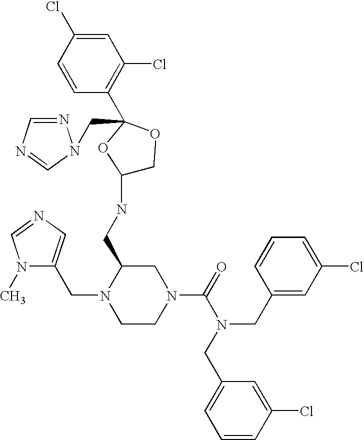 | <0.1 | <10 | <10 |
| 10 | 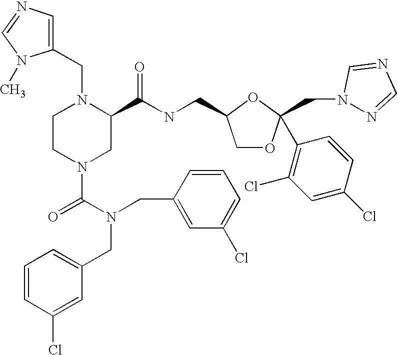 | <0.1 | <10 | <10 |
| 11 | 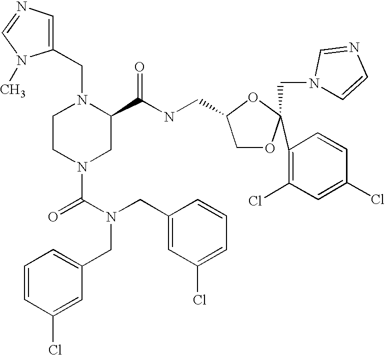 | <0.1 | <10 | <10 |
| 12 | 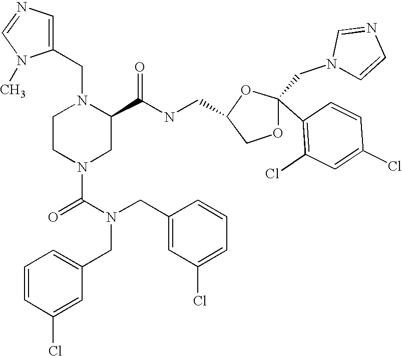 | <0.1 | <10 | <10 |
| 13 | 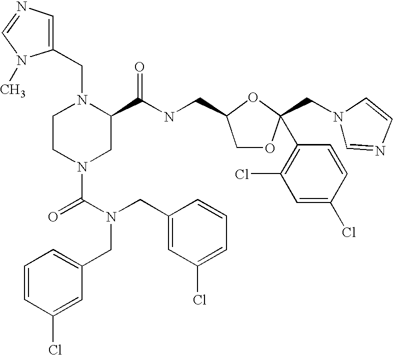 | <0.1 | <10 | <10 |
| 14 | 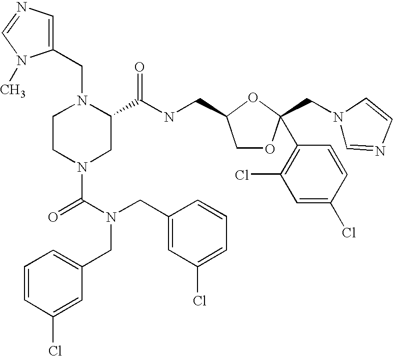 | <0.1 | <10 | <10 |
| 15 | 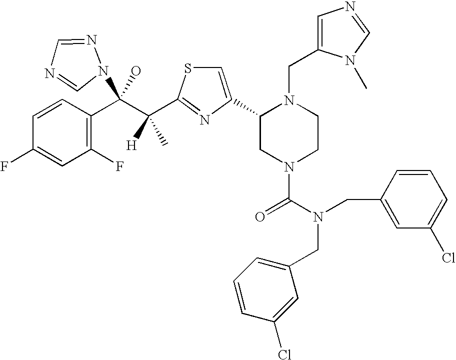 | <0.1 | <10 | <10 |
| 16 | 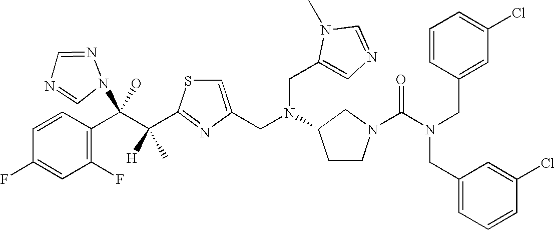 | <0.1 | <10 | <10 |

Inhibition of CAK1

The following protocol was used for measuring CAK1 inhibition by test compounds:

A compound plate was pre-made with 20 μg/ml compound dissolved in 100% DMSO in each well of a 384-well plate (polypropylene, Coming cat # 29445-136), except for two control columns, wherein each well contained 100% DMSO.

A substrate plate was prepared by adding a pre-determined volume of substrate solution (925 nM biotin-his-cdk2 in reaction buffer) into each well of a 96-well deep-well plate (Beckman cat. 267006). To the control wells of the plate, 1/20 vol. of positive control and blank solutions were added according to the plate map. Positive control solutions are Olomoucine (MTX-277859) at 0.25, 0.75, 2.25, 6.75 mM, respectively, in DMSO, and blank solution is 50 mM EDTA in reaction buffer (50 mM Tris-HCl (pH 7.5), 50 mM NaCl, 0.1 mg/mL BSA, 100 μM MgCl₂).

The reaction plate (384-well polystyrene black, Packard Opti plate, Cat.# 6005256) was loaded with a volume of 2.0 μL of samples from the compound plate and 8.0 μL of substrate solution from the substrate plate. 10 μl of enzyme solution (10 nM his-CAK1, 20 μM ATP in reaction buffer) was added to start the reaction. The plate was covered with a lid and incubated at room temperature for 30 min.

10 μl of FRET solution (45 nM streptavidin-APC*, 30 nM Europium-Ab, 3 mM EDTA in reaction buffer) was added to each of the wells of the reaction plate to stop the reaction. The time-resolved fluorescence was measured on a Wallac Victor fluorometer (excitation 340 nm, emission 665 nm, delay time 100 μs, window time 100 μs).

Table 2 presents compounds identified in the above assay:

TABLE 2

| No. | IC50 (μM) | Structure |
|---|---|---|
| 100 | <50 | 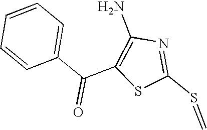 |
| 101 | <50 | 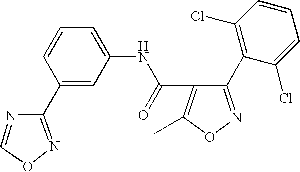 |
| 102 | <10 | 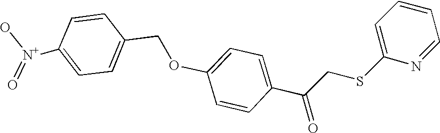 |
| 103 | <50 | 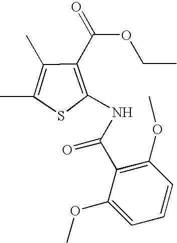 |
| 104 | >50 | 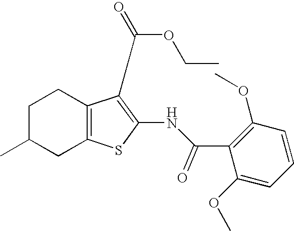 |
| 105 | <50 | 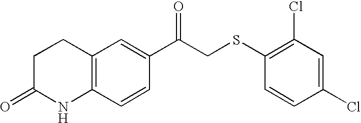 |
| 106 | <50 | 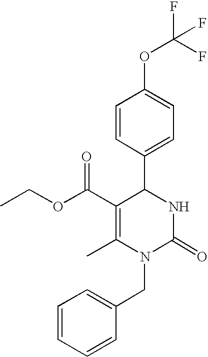 |
| 107 | <10 | 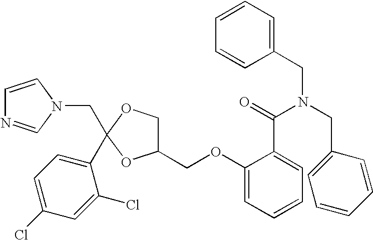 |
| 108 | <50 | 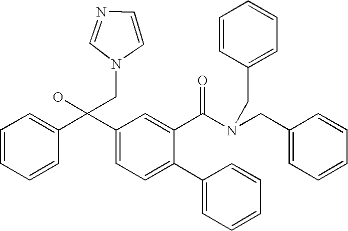 |

All of the references and publications cited herein and U.S. application Ser. Nos. 09/305,929 and 08/631,319 are hereby incorporated by reference.

Equivalents

Those skilled in the art will recognize, or be able to ascertain using no more than routine experimentation, numerous equivalents to the compounds and methods of use thereof described herein. Such equivalents are considered to be within the scope of this invention and are covered by the following claims.

1. A method for preparing a cytotoxic antifungal agent, comprising
i) identifying a small organic molecule having a core structure, the small organic molecule having a cytotoxic antifungal activity; and
ii) preparing a compound comprising the core structure of the identified cytotoxic antifungal agent and a subunit having the formula: 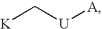
wherein
A represents a substituted or unsubstituted aryl or heteroaryl ring;
U represents a carbon or nitrogen atom to which a linkage to the core structure is attached; and
K represents a nitrogen-containing heteroaryl ring.

2. The method of claim 1, wherein K represents an imidazole or triazole ring.

3. The method of claim 1, wherein A represents a phenyl ring.

4. The method of claim 3, wherein the phenyl ring is substituted.

5. The method of claim 4, wherein the phenyl ring is substituted at an ortho and is a para position.

6. The method of claim 5, wherein the phenyl ring is substituted with a halogen at each of the ortho and para positions.

7. The method of claim 2, wherein A represents a phenyl ring.

8. The method of claim 7, wherein the phenyl ring is substituted.

9. The method of claim 8, wherein the phenyl ring is substituted at an ortho and a para position.

10. The method of claim 9, wherein the phenyl ring is substituted with a halogen at an ortho and a para position.

11. The method of claim 1, wherein the core structure consists essentially of the atoms of the identified agent.

12. The method of claim 1, wherein the core structure is shared by at least ten compounds identified as having a cytotoxic antifungal activity.

13. The method of claim 1, wherein the core structure is shared by at least thirty compounds identified as having a cytotoxic antifungal activity.

14. The method of claim 1, wherein the core structure is shared by at least seventy-five compounds identified as having a cytotoxic antifungal activity.

15. The method of claim 1, wherein the cytotoxic antifungal activity is selected from N-myristoyltransferase inhibitory activity, prenyltransferase inhibitory activity, and CDK-activating kinase 1 (CAK1) inhibitory activity.

16. A cytotoxic antifungal agent having a structure comprising
i) an active portion that, taken alone, is a small organic molecule with cytotoxic antifungal activity; and
ii) a subunit having the formula: 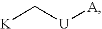
wherein
A represents a substituted or unsubstituted aryl or heteroaryl ring;
U represents a carbon or nitrogen atom to which a linkage to the core structure is attached; and
K represents a nitrogen-containing heteroaryl ring.

17. The agent of claim 16, wherein K represents an imidazole or triazole ring.

18. The agent of claim 16, wherein A represents a phenyl ring.

19. The agent of claim 18, wherein the phenyl ring is substituted

20. The agent of claim 19, wherein the phenyl ring is substituted at an ortho and a para position.

21. The agent of claim 20, wherein the phenyl ring is substituted with a halogen at an ortho and a para position.

22. The agent of claim 17, wherein A represents a phenyl ring.

23. The agent of claim 22, wherein the phenyl ring is substituted.

24. The agent of claim 23, wherein the phenyl ring is substituted at an ortho and a para position.

25. The agent of claim 24, wherein the phenyl ring is substituted with a halogen at each of the ortho and para positions.

26. The agent of claim 16, wherein the cytotoxic antifungal activity is selected from N-myristoyltransferase inhibitory activity, prenyltransferase inhibitory activity, and CDK-activating kinase 1 (CAK1) inhibitory activity.

27. A pharmaceutical preparation comprising a sterile excipient and the agent of claim 16.

28. A method for inhibiting growth or proliferation of a fungal cell, comprising contacting the fungal cell with the agent of claim 16.

29. A method for treating a patient having a fungal infection, comprising administering to the patient the agent of claim 16.

30. The method of claim 29, wherein the patient is a human.
